(19) United States
(12) Patent Application Publication
(10) Pub. No.: US 2018/0243418 A1
(43) Pub. Date: Aug. 30, 2018

(54) SYSTEMS AND METHODS FOR TARGETED CANCER THERAPIES

(71) Applicant: American University of Sharjah, Sharjah (AE)

(72) Inventors: Ghaleb Husseini, Sharjah (AE); Mohammad Al-Sayah, Sharjah (AE); Ana Martins, Berkeley, CA (US); Rute Vitor, Sharjah (AE); Najla Salkho, Sharjah (AE)

(21) Appl. No.: 15/703,675

(22) Filed: Sep. 13, 2017

Related U.S. Application Data
(60) Provisional application No. 62/464,294, filed on Feb. 27, 2017.
(60) Provisional application No. 62/464,325, filed on Feb. 27, 2017.
(60) Provisional application No. 62/482,572, filed on Apr. 6, 2017.

(51) Int. Cl. A61K 41/00; A61K 9/00; A61K 47/68; A61K 47/69; A61K 9/127; A61H 23/02; A61N 7/00; B06B 1/02; C07J 1/00

(52) U.S. Cl. CPC A61K 41/0033; A61K 9/0009; A61K 47/6889; A61K 47/6913; A61K 9/1271; A61H 23/0245; A61N 2007/0043; B06B 1/0276; C07J 1/0051; A61N 2007/0073; A61N 2007/0039; A61N 2007/0004; A61N 7/00

(57) ABSTRACT

Systems and methods for producing liposomes, including control liposomes and various targeted liposomes. Systems and methods for treating conditions, such as, but not limited to various cancers, using targeted liposomes produced according to various methods disclosed herein. For example, estrone-conjugated liposomes may be used deliver chemotherapeutic agent(s) to breast cancer tissues for the treatment of breast cancer. Systems and methods for actuating liposomes using ultrasound. For example, systems and methods for actuating estrone-conjugated liposomes accumulated in breast cancer tissues for the treatment of breast cancer.

### INCORPORATION BY REFERENCE TO ANY PRIORITY APPLICATIONS

This application claims priority benefit of U.S. Provisional Application Nos. 62/464,294 (Dkt. No. AUS.001PR), filed on Feb. 27, 2017, 62/464,325 (Dkt. No. AUS.002PR), filed on Feb. 27, 2017, and 62/482,572 (Dkt. No. AUS.003PR), filed on Apr. 6, 2017. All of the above applications are incorporated by reference herein and are to be considered a part of this specification. Any and all applications for which foreign or domestic priority claim is identified in the Application Data Sheet as filed with the present application are hereby incorporated.

### BACKGROUND

### Field

The present application relates generally to systems and methods for producing acoustically activated or triggered nanoparticles and more specifically, relates to systems and methods for producing and using acoustically activated or triggered, ligand-targeted liposomes for the novel treatment of cancer.

### Description of the Related Art

Cancer is a disease commonly understood to be caused by an abnormal, uncontrolled cell growth, which may invade neighboring and/or surrounding tissues and organs. Neoplasms and malignant tumors are other terms used to name cancer. Cancer can be grouped into at least the following categories: carcinoma, which includes cancers that start in the skin or tissues that cover organs; sarcoma, which includes cancers occurring in connective or supportive tissues (e.g., bone and muscles); leukemia, which includes cancers that originate in blood forming tissue; lymphoma and myeloma, which includes cancers affecting the cells of the immune system; and central nervous system cancers.

Cancer, which may be (and frequently is) a lethal disease, has been cited as the second most common cause of death in the USA. Fifteen million cancer cases are predicted to be diagnosed by the year 2020, and about twelve million cancer patients are expected to die. Table 1, below, provides select 2012 cancer statistics in the United States. As can be seen, the three most commonly diagnosed cancers among men are prostate, lung, and colorectal cancers, whereas, breast, lung, and colorectal cancers are the most commonly diagnosed types among women. In addition, men may have a higher overall probability of being diagnosed with cancer compared to women.

TABLE 1
Cancer Statistics, United States, 2012

| Comparison | Women | Men |
|---|---|---|
| Most Commonly | 1. Breast | 1. Prostate |
| Diagnosed Cancers | 2. Lung and Bronchus | 2. Lung and Bronchus |
| | 3. Colorectal | 3. Colorectal |
| Lifetime Probability to | Lower | Higher |
| be Expected with Cancer | | |

Cancer treatments are frequently grouped into four categories, including: 1) surgery, 2) chemotherapy, 3) radiation, and 4) antibody blocking therapy (these four treatment schedules may be and frequently are combined in an effort to achieve an improved outcome). For example, more selective and effective cancer therapies may comprise a combination of chemotherapy, radiation, and antibody blocking therapy, to reduce the impact of chemotherapeutic drugs on healthy cells.

### SUMMARY

In some embodiments, a method of treating breast cancer in a patient comprises inserting into the body of the patient a first quantity of an actively targeted liposome, allowing the actively targeted liposomes to circulate throughout a circulatory system of the patient for a time, wherein the time is sufficiently long to allow aggregation of a second quantity of the actively targeted liposomes at a treatment area comprising the breast cancer, wherein the second quantity of the actively targeted liposomes is therapeutically significant, and applying ultrasound to the treatment area comprising the breast cancer and critically disrupting a third quantity of the actively targeted liposome so that the chemotherapeutic drug is released in the treatment area. The actively targeted liposome may comprise a lipid bilayer forming a spherical shell, wherein the spherical shell defines an interior liposomal cavity, estrone linked to a surface of the actively targeted liposome using cyanuric chloride, and a chemotherapeutic drug, wherein the chemotherapeutic drug comprises at least one of a hydrophilic chemotherapeutic drug contained within the interior liposomal cavity and a hydrophobic chemotherapeutic drug contained within the lipid bilayer of the actively targeted liposome. The actively targeted liposomes comprise sonosensitive large unilamellar vesicles;

The ultrasound applied to the treatment area may comprise a low frequency ultrasound. The low frequency ultrasound may comprise a 20 kHz ultrasound having at a power density of one of 6.08 W/cm2, 6.97 W/cm2, and 11.83 W/cm2. The low frequency ultrasound applied to the treatment area may be applied for less than about 10 minutes. The ultrasound applied to the treatment area may comprise a high frequency ultrasound. The high frequency ultrasound may comprise a 1.07 MHz ultrasound having at a power density of one of 10.5 W/cm2, 50.2 W/cm2, and 173 W/cm2. The high frequency ultrasound may comprise a 3.24 MHz ultrasound having at a power density of one of 10.5 W/cm2, 50.2 W/cm2, and 173 W/cm2. The high frequency ultrasound applied to the treatment area may be applied for less than about 10 minutes.

In some embodiments, a method of treating a cancer in a patient comprises inserting a quantity of an actively targeted nanoparticle in a body of the patient, the actively targeted nanoparticle comprising a lipid bilayer and a drug formulation within the actively targeted nanoparticle, wherein at least a portion of an external surface of the lipid bilayer is functionalized with a targeting moiety, allowing at least a portion of the quantity of the actively targeted nanoparticle to aggregate at a treatment site in the patient, wherein the treatment site comprises the cancer, and applying ultrasound to the treatment site in the patient.

The drug formulation may comprise a hydrophobic drug contained within the lipid bilayer. The drug formulation may comprise a hydrophilic drug contained in a core of the actively targeted nanoparticle. The targeting moiety may comprise an estrogen hormone. The estrogen hormone may comprise estrone. The targeting moiety may comprise at least one of RGD, transferrin, and plasminogen. The actively targeted nanoparticle may be PEGylated. The method may include applying ultrasound to the treatment site in the patient to induce critical disruption of the lipid bilayer of a portion of the quantity of the actively targeted nanoparticle aggregated at the treatment site in the patient thereby releasing the drug formulation contained within the portion of the quantity of the actively targeted nanoparticle aggregated at the treatment site. The ultrasound applied to the treatment site may comprise a low frequency ultrasound applied for less than about 20 minutes. The low frequency ultrasound may comprise a 20 kHz ultrasound having at a power density of one of 6.08 W/cm2, 6.97 W/cm2, and 11.83 W/cm2. The ultrasound applied to the treatment site may comprise a high frequency ultrasound applied for less than about 20 minutes. The high frequency ultrasound may comprise a 1.07 MHz ultrasound having at a power density of one of 10.5 W/cm2, 50.2 W/cm2, and 173 W/cm2. The high frequency ultrasound comprises a 3.24 MHz ultrasound having at a power density of one of 10.5 W/cm2, 50.2 W/cm2, and 173 W/cm2.

### BRIEF DESCRIPTION OF THE DRAWINGS

FIG. 1 shows schematics of normal and cancerous cellular growth pathways.

FIGS. 2A-C show schematic representations of liposomes incorporating one or more of hydrophobic drugs, hydrophilic drugs, and ligands or moieties. FIG. 2A shows a liposome incorporating a hydrophobic drug within the lipid bilayer. FIG. 2B shows a liposome encapsulating a hydrophilic drug within its center. FIG. 2C illustrates a liposome modified with a ligand or moiety (attached to its surface).

FIGS. 3A-3B show a liposome and its constituents. FIG. 3A shows a phospholipid building block. FIG. 3B shows a cut-away of a liposome.

FIGS. 4A-4C show examples of various structures of liposomes.

FIGS. 5A-5C show examples of various sizes of liposomes.

FIGS. 6A-6C show cut-away views of various liposomes. FIG. 6A shows a cut away view of a standard liposome having a lipid bilayer and an aqueous core. FIG. 6B shows a cut away view of a PEGylated liposome (stealth liposome) having a lipid bilayer, PEG attached to its surface, and an aqueous core. FIG. 6C shows a cut away view of a functionalized or targeted stealth liposome having a targeting moiety attached to the ends of the PEG.

FIGS. 7A-7C show various structures formed out of phospholipids. FIG. 7A shows a liposome. FIG. 7B shows a micelle. FIG. 7C shows a planar lipid bilayer.

FIGS. 8A-8G show examples different techniques used to attach antibodies to lipoma surfaces. FIG. 8A shows surface adsorption. FIG. 8B shows covalent coupling of the antibody. FIG. 8C shows PEG spacers. FIG. 8D shows hapten binding. FIGS. 8E and 8G show avidin-biotin with either avidin or biotin attached to the surface.

FIGS. 9A-9D show mechanisms of interaction between cells and liposomes. FIG. 9A shows absorption. FIG. 9B shows fusion. FIG. 9C shows receptor-mediated endocytosis. FIG. 9D shows phagocytosis.

FIG. 10A-10B show the structure of human estrogen receptors ERα and ERβ. FIG. 10A shows the structure of ERα. FIG. 10B shows the structure of ERβ

FIGS. 11A-11D show ER signaling mechanisms. FIG. 11A shows the classical mechanism of ER action. FIG. 11B shows the ER-independent genomic mechanism. FIG. 11C shows the ligand-independent genomic mechanism. FIG. 11D shows non-genomic action.

FIG. 12 shows the 2D molecular structure of estrone.

FIGS. 13A-13E show various schematic representations of the piezoelectric effect. FIG. 13A shows a piezoelectric bar. FIG. 13B shows a piezoelectric bar with a voltage applied reverse to the polarization of the bar. FIG. 13C shows a piezoelectric bar with a voltage applied in the same direction as the polarization of the bar. FIG. 13D shows a measure of the voltage drop across the piezoelectric bar when the bar is under compression. FIG. 13E shows a measure of the voltage drop across the piezoelectric bar when the bar is under tension.

FIG. 14 shows a schematic representation of US energy deposition.

FIG. 15 shows a schematic of the synthesis of estrone-cyanuric derivative.

FIG. 16 shows a schematic of the synthesis of DSPE-PEG-pNP lipids.

FIG. 17 shows a schematic of the preparation of calcein-containing DSPE-PEG-pNP liposomes from DPPC, cholesterol and DSPE-PEG-pNP.

FIG. 18 shows a schematic of the synthesis of DSPE-PEG₂₀₀₀-N₃C₃Cl-ES.

FIG. 19 shows a schematic of the preparation of DSPE-PEG-Albumin liposomes.

FIG. 20 shows an apparatus that may be used to evaluate liposome release.

FIG. 21 shows an example online release profile produced using pulsed mode ultrasound.

FIG. 22 shows example offline release profiles for various times after 60 minutes of insonation with a 10 minute interval.

FIG. 23 shows an IR spectra of estrone and cyanuric chloride before the reaction (ES+(NCCL)₃) and the formed conjugate (ES-N₃C₃Cl₂) after the reaction.

FIG. 24 shows the average radii of DSPE-PEG-pNP control liposomes and albumin liposomes. The results were obtained by DLS measurements and the average±standard deviation of different technical replicates are shown for 3 batches of each liposome type.

FIG. 25 shows a typical normalized online LFUS release curve from targeted liposomes.

FIG. 26 shows normalized release profiles for DSPE-PEG₂₀₀₀-NH₂ liposomes triggered by 20-kHz LFUS at 8.08, 6.97, and 11.83 W/cm². Results are average±standard deviation of two liposome batches (3 replicates each).

FIG. 27 shows normalized release profiles for DSPE-PEG₂₀₀₀-NH₂ liposomes triggered by 20-kHz LFUS at 8.08, 6.97, and 11.83 W/cm². Results are average±standard deviation of two liposome batches (3 replicates each) and are shown for first two pulses in addition to final release.

FIG. 28 shows calcein release curves from DSPE-PEG-pNP control liposomes, triggered by 20 kHz pulsed ultrasound (20 s on 10 s off) at 8.08, 6.97, and 11.83 W/cm². Results are average±standard deviation of different liposome batches for each power density. Error bars are included.

FIG. 29 shows a comparison of the initial release of calcein from control DSPE-PEG-pNP at 8.08, 6.97, and 11.83 W/cm².

FIG. 30 shows a comparison of the final release percentage of calcein from control DSPE-PEG-pNP at 8.08, 6.97, and 11.83 W/cm².

FIG. 31 shows normalized release profiles for DSPE-PEG₂₀₀₀-N₃C₃Cl-ES liposomes triggered by 20-kHz LFUS at 8.08, 6.97, and 11.83 W/cm². Results are average±standard deviation of two liposome batches (3 replicates each).

FIG. 32 shows normalized release profiles for DSPE-PEG₂₀₀₀-NH₂ liposomes triggered by 20-kHz LFUS at 8.08, 6.97, and 11.83 W/cm². Results are average±standard deviation of two liposome batches (3 replicates each) and are shown for first two pulses in addition to final release.

FIG. 33 shows calcein release curves from DSPE-PEG-albumin liposomes, triggered by 20 kHz pulsed ultrasound (20 s on 10 s off) at 8.08, 6.97, and 11.83 W/cm². Results are average±standard deviation of different liposome batches for each power density. Error bars are included.

FIG. 34 shows a comparison of the initial release rates of calcein from DSPE-PEG-albumin liposomes at different power densities.

FIG. 35 shows a comparison of the final release percentage of calcein from DSPE-PEG-albumin liposomes at different power densities.

FIGS. 36A-36C show various comparisons of the normalized calcein release profiles from DSPE-PEG₂₀₀₀-N₃C₃Cl-ES and DSPE-PEG₂₀₀₀-NH₂ liposomes. FIG. 36A shows the release profiles when the liposomes are triggered by 20-kHZ LFUS at 6.08 W/cm². FIG. 36B shows the release profiles when the liposomes are triggered by 20-kHZ LFUS at 6.97 W/cm². FIG. 36C shows the release profiles when the liposomes are triggered by 20-kHZ LFUS at 11.83 W/cm².

FIGS. 37A-37C show various comparisons of calcein release from DSPE-PEG₂₀₀₀-N₃C₃Cl-ES and DSPE-PEG₂₀₀₀-NH₂ liposomes triggered by 20-kHz at 6.08, 6.97, and 11.83 W/cm². FIG. 37A shows release after the first ultrasound pulse. FIG. 37B shows release after the second ultrasound pulse. FIG. 37C shows the final release.

FIGS. 38A-38C show various comparisons of the release curves for control and albumin liposomes. FIG. 38A shows a comparison at 6.08 W/cm². FIG. 38B shows a comparison at 6.97 W/cm². FIG. 38C shows a comparison at 11.83 W/cm². Results are average±standard deviation of different liposome batches for each power density. Error bars are included.

FIGS. 39A-39B show various comparisons of calcein release from DSPE-PEG-albumin liposomes at different power densities. FIG. 39A shows release after the first ultrasound pulse. FIG. 39B shows the final release.

FIG. 40 shows normalized calcein release profiles from DSPE-PEG₂₀₀₀-NH₂ liposomes, triggered by 1.07 and 3.24 MHz HFUS, at power density of 10.5 and 50.2 W/cm² and 3.24 W/cm², respectively. Results are average±standard deviation of 2 liposome batches (3 replicates).

FIG. 41 shows normalized calcein release profiles from DSPE-PEG₂₀₀₀-N₃C₃Cl-ES liposomes, triggered by 1.07 and 3.24 MHz HFUS, at power density of 10.5 and 50.2 W/cm² and 3.24 W/cm², respectively. Results are average±standard deviation of 2 liposome batches (3 replicates).

FIG. 42 shows comparisons of the normalized calcein release profiles from DSPE-PEG₂₀₀₀-N₃C₃Cl-ES and DSPE-PEG₂₀₀₀-NH₂ liposomes triggered by 1.07 and 3.24 MHz HFUS, at power density of 10.5 and 50.2 W/cm² and 3.24 W/cm², respectively. Results are average±standard deviation of 2 liposome batches (3 replicates).

FIG. 43 shows a comparison of calcein uptake by ER-negative cells versus the calcein uptake of ER-positive cells.

FIG. 44 shows a comparison of calcein uptake by ER-positive cells before and after sonication.

FIG. 45 shows an RGD-modified liposome schematic with encapsulated calcein.

FIG. 46 shows an HPLC scan revealing successful liposomal functionalization with RGD.

FIG. 47 shows the release of calcein over time from RGD functionalized liposomes.

### DETAILED DESCRIPTION

The combined number of new cancer cases and cancer deaths in the United States in 2014 for men and women is estimated to be about 1,665,540 and 585,720, respectively (for all types of cancer, except basal cell and squamous cell skin cancers). Accordingly, cancer is the second major cause of death in the United (following heart diseases).

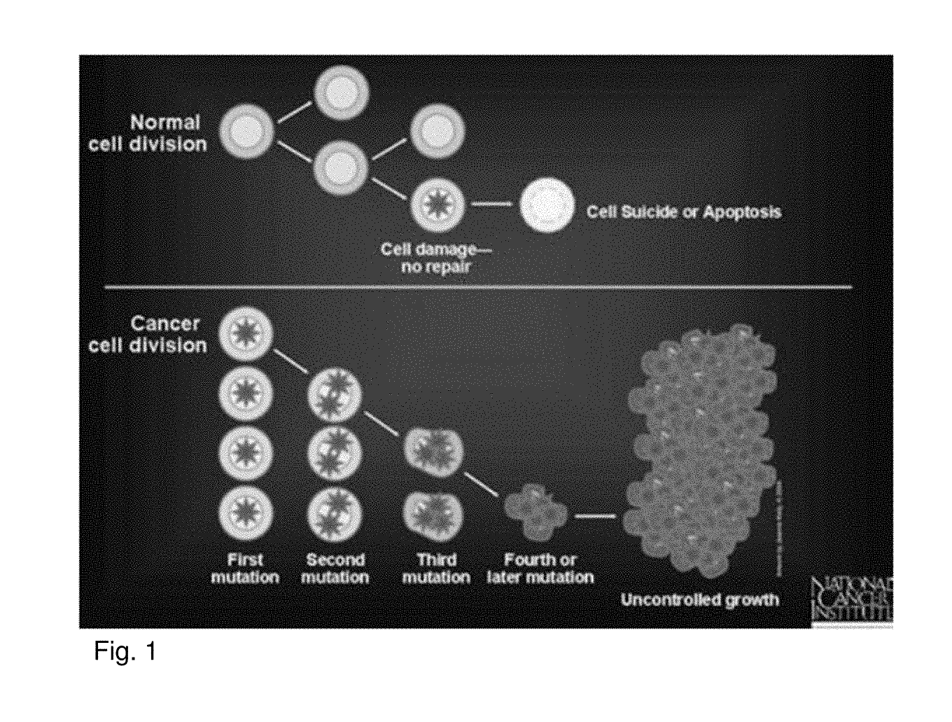

Cancer, also known as a malignant tumor, is a disease caused by abnormal, uncontrolled cell growth. Cancer starts in the body's basic unit of life, the cell. Cancerous cells may spread either by invasion or metastasis. By contrast, benign tumors are localized and less threatening than cancer. Normally, healthy cells follow a regular progression of growth, division, and apoptosis (programmed cell death). Unlikely healthy cells, cancer cells do not undergo proper apoptosis. Instead, cancer cells continue to growing and divide (e.g., even after they should have apoptosed) due to mutation(s), e.g., DNA mutations, that hinder the function of genes involved in cell division. FIG. 1 shows example pathways of cellular division of both normal cells and cancerous cells.

Genes responsible for cell division are generally grouped into the following four types: (1) suicide genes, which may control apoptosis; (2) oncogenes, which may determine when cells should divide; (3) tumor suppressor genes, which may regulate one or more of transcription, cell division, cell differentiation, and cell death; and (4) DNA-repair genes, which may repair damaged DNA.

Some treatments for cancer include surgery (e.g., radical resection), chemotherapy, hyperthermia, radiation, and targeted therapies (and various combinations of the aforementioned). Combinations of different modalities may beneficially improve treatment outcomes. For instance, adjuvant chemotherapy, e.g., a chemotherapy regimen undergone after a surgery (such as radical resection), may significantly improve cancer treatment outcomes. Targeted therapy, alone or in combination with one or more other cancer therapies, has advanced the standard of care and may provide cancer patients with better relief and fewer undesirable side effects.

Chemotherapeutic agents can treat many types of cancer, since they consists of antineoplastic agents of diverse different classes and modes of action. Chemotherapy uses cytotoxic (toxic to living cells) drugs to destroy cancer cells (e.g., tumor tissue), or to slow their growth/division. Cytotoxic drugs are generally classified as: (1) alkylators (the largest group), such as, but not limited to, Melphalan (also known as Alkeran); (2) topoisomerase inhibitors, such as, but not limited to, doxorubicin (Dox); (3) antimetabolites, such as, but not limited to, Caldribine; (4) microtubule interacting agents, such as, but not limited to, Vinorelbine; and (5) amino acid depletion agents, such as, but not limited to, asparaginase. These types of cytotoxic drugs differ in the mechanism by which they destroy and/or slow the growth/division of cancer cells. For example, asparaginase hydrolyzes asparagine, an amino acid essential for cell and tumor growth, therefore, it affects the cellular membrane of certain types of cancer that are unable to synthesize their own asparagine (and depend on circulating asparagine for survival). On the other hand, most cytotoxic chemicals, such as, but not limited to, alkylators, interact with and preclude intercellular functions needed for cell survival. Due to their cytotoxic properties, frequent administration of cytotoxic drugs may have harmful side effects. For example, frequent administration of doxorubicin may result in cardiac dysfunction and possible death.

Chemotherapy may be administrated in many ways, including, but not limited to, intravenously, orally, dermally (e.g., into the skin), and or transdermally (e.g., direct injection into an organ or other tissue). Chemotherapy can provide a cure/remission to some cancer patients, such as those suffering from acute leukemia. However, it may not necessarily guarantee a cure, or other positive outcome, for some cancer types. Chemotherapy may improve the survival rate of some patients, particularly those suffering from breast or colorectal cancers. Furthermore, chemotherapy may only provide tumor-associated symptoms of relief, as may be observed with pancreatic cancers. In other cases chemotherapy has little beneficial effect, such as in the case of thyroid cancer, which can be relatively insensitive to chemotherapy.

Chemotherapy has many potential downsides, especially when administered by conventional methods, when it affects not only cancer cells, but also healthy (normal) cells. In fact, cytotoxic agents generally do not distinguish between normal cells and malignant cells, and affect both equally. As a result, patients receiving chemotherapy often experience uncomfortable or even life threatening serious side-effects. Chemotherapeutic drugs frequently target fast growing cells (e.g., have the most significant effect on rapidly dividing or replicating types of cells) including, but not limited to tumor cells, hair follicles, cells lining the mouth, and the digestive track; consequently, some common side effects of chemotherapy include hair loss, nausea, vomiting, throat and mouth sores, and diarrhea. Fatigue and loss of appetite are other common side effects, though they may not, necessarily, be tied to chemotherapy's selectivity for rapidly replicating cells. In addition, chemotherapy may affect the bone marrow, which, as a result, may hinder the production of red blood cells, white blood cells, and platelets. This can lead to an increased susceptibility to infections and/or risk of bleeding. Beyond the potential side effects of cytotoxic agents, 70% of the patients who undergo chemotherapy do not respond effectively to the initial administration of the anti-neoplastic agent. As a result, many patients develop (e.g., build up) a resistance after several administrations of a drug; a phenomenon caused by the cellular multi-drug resistance (MDR). The many harmful and/or life threatening side effects of the conventional chemotherapy medication(s), such as cardiotoxicity, and the MDR effect, is one reason underlying the development of new/different pharmacological medication approaches or drug delivery regimes, including so called “targeted therapies, which aim to improve the specificity and reduce both the deleterious side effect and the invasiveness of conventional cancer therapies.

Targeted therapies are intended/designed to selectively attack cancer cells, while minimizing their effect on normal, healthy cells. For example, some targeted therapies may attack a specific target in the cell to inhibit the cell's growth pathways. Targeted therapies may have a mechanism of action based on blocking growth factor receptors (which tend to be overexpressed in cancer cells), and, therefore, blocking the signals responsible for uncontrolled cancer growth (and/or uncontrolled cellular replication). Targeted therapies may be classified into two groups, including: (1) therapeutic monoclonal antibodies; and (2) small molecules. Therapeutic monoclonal antibodies have certain properties that may render them particularly advantageous in targeted cancer therapies. For example, antibodies may be more targeted (e.g., most specifically targeted) than small molecules since they bind to particular and/or select antigens located on the exterior surface of the cell. Additionally, antibody-based drugs may have a comparatively long half-life (e.g., 2-3 weeks) that allows them to treat chronic diseases.

Drug delivery systems (DDS) using nanoparticles (and/or microparticles) may be used as drug carriers in targeted (or untargeted) therapies. Nanoparticles/nanocarriers (which, depending on size may be microparticles/microcarriers) are a distinct collection of molecules ranging in size from about 1 nm to 1 μm. Nanocarriers may encapsulate a variety of therapeutic agents and/or targeted therapies, including, but not limited to small hydrophilic/hydrophobic molecules, peptides, and nucleic acids. The encapsulated molecules can then be released from the carrier in a controlled manner over a given period of time. Furthermore, due to the small size of the nanocarriers, transport across biological barriers can be enhanced, hence facilitating cellular uptake. In addition, the surface of nanocarriers can be modified to increase the blood circulation half-life. Another advantage of using nanoparticles is that they may be biodegradable. Nanocarriers may be designed to be highly specific, allowing the controlled targeting of cancer cells, thus decreasing the cytotoxicity of the therapeutic agent on healthy cells. They may also contribute to a reduction in chemotherapy cost and potential side effects on the patient's organs. Another advantage of using nanocarriers is that they may have a higher therapeutic index for the agent at the tumor, which can improve imaging and detection of tumors when carrying imaging agents or in addition to a drug.

Using carefully designed nanoparticles, it may be possible to control the time and space in which a therapeutic agent is released. For example, control over the space/volume in which a therapeutic agent is released may be achieved using nanoparticles targeted to a certain type of cancer cells (e.g., selective to a particularly characteristic of a given type of cancer, such as, but not limited to, an over-expressed receptor). After accumulation at a tumor site, release of the encapsulated therapeutic agents may be triggered by applying a stimulus to which the nanoparticles respond. For example, nanoscale therapeutic carriers can be combined with ultrasound (US) as a triggering modality, to deliver cytotoxic agents to specific tissues or cells using an appropriate ultrasonic frequency, power density and/or other acoustic factors. The technique of using nanocarriers in conjunction with a trigger can enhance pharmacological properties by modifying (e.g., improving) drug pharmacokinetics and biodistribution. The nanoparticles used in DDS may include micelles, liposomes, dendrimers, and various polymeric-based systems.

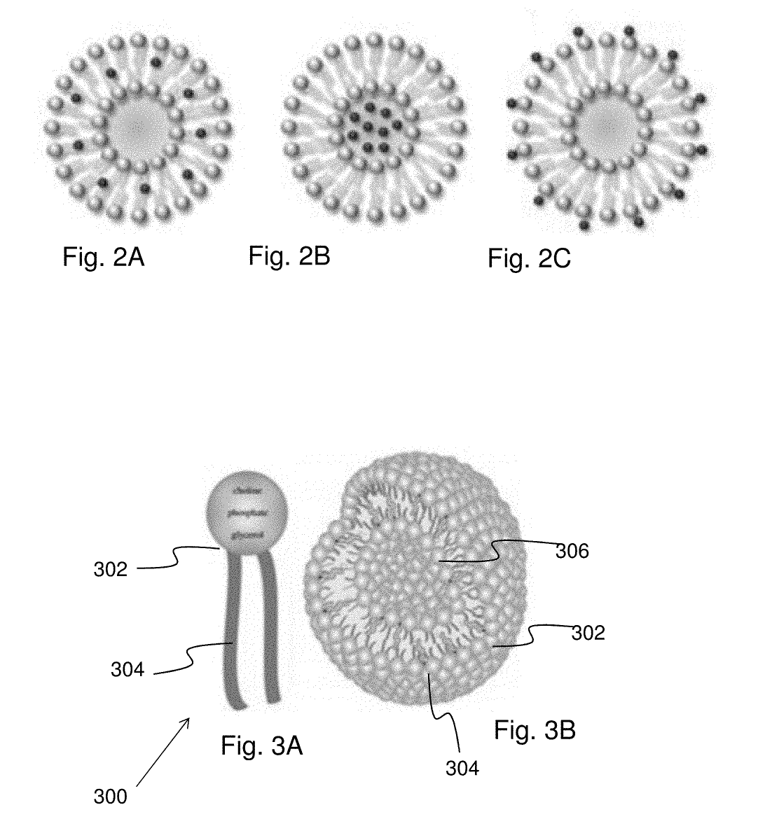

Liposomes have certain advantages that may make them particularly well-suited to drug delivery systems. Liposomes are vesicles composed of lipid bilayer(s) (e.g., one or more bilayers) that surround(s) an aqueous core. Liposomes generally have a diameter in the range of between about 0.05-1 μm. However, other sizes of liposomes are contemplated by this disclosure, including, but not limited to liposomes in the range of between about 0.01-2 μm, between about 0.05-1.9 μm, between about 0.1-1.8 μm, between about 0.2-1.7 μm, between about 0.3-1.6 μm, between about 0.4-1.5 μm, between about 0.5-1.4 μm, between about 0.6-1.3 μm, between about 0.7-1.2 μm, between about 0.8-1.1 μm, between about 0.9-1 μm, or any other size of liposome that may be desirable (or that may be formed) according to or in any method disclosed herein. Due to the hydrophobic properties and structure of lipid bilayers, hydrophobic drugs can be carried within the lipid bilayer. An example of a liposome carrying a hydrophobic drug within its lipid bilayer is shown in FIG. 2A. As shown in FIG. 2B, hydrophilic drugs can be encapsulated in the inner, aqueous interior of the liposome.

In addition to drugs carried within the liposome (e.g., hydrophobic drugs carried within the lipid bilayer and/or hydrophilic drugs carried within the aqueous core), liposomes may be “functionalized” or “targeted” with ligands or other moieties, such as is shown in FIG. 2C. For example, some cancer cells may overexpress receptors for albumin on their surface; hence, liposomes designed to target these cancer cells may be functionalized with a human serum albumin (HAS) moiety conjugated to an outer surface of the liposome. Similarly, almost two-thirds of breast cancer overexpress estrogen receptors. Moreover, the binding of estrogen hormones to estrogen receptors in cancer cells increases cell division and leads to DNA mutation. Therefore, estrone-anchored (estrone is a steroidal hormone with various functionalities) liposomes may be used to target cancer cells (e.g., breast cancer cells) that overexpress estrogen receptors (ERs).

The release of encapsulated molecules from within liposomes may be triggered in any of a number of ways, such as by local application of energy, including, but not limited to, ultrasound (US). Various applications of ultrasound in medicine are well known and include diagnostic imaging, kidney stone disruption, blood flow analysis, and tumor ablation. Ultrasound frequencies used for such, common applications usually range between about 0.8-3 MHz. In actuated drug delivery or targeted therapies, such as those disclosed herein, ultrasound may be used as a triggering technique to induce catastrophic disruption of targeted/functionalized liposomes and thereby enhance drug release to specific cancer cells. Various phenomena induced by ultrasound, including, but not limited to, hyperthermia (temperature effects) and cavitation (mechanical effects), are advantageously exploited in one or more of the methods disclosed herein.

### Drug Delivery System

Chemotherapeutic agents can suppress tumor growth. However, they generally target both normal (e.g., healthy) and cancer cells. Also, the dose of chemotherapy, which may be important (e.g., may be critical), is bound by two levels, known as therapeutic indices: (1) the maximum level for which the patient experiences undesirable effects if exceeded (otherwise stated, the amount of the drug that causes toxicity), and (2) the minimum level where the drug efficacy is not attained (otherwise stated, the lowest level that causes the intended therapeutic effect). The therapeutic indices, which bound the therapeutic window, are also known as the toxic dose and the effective dose. Any drug should, ideally, be delivered at a dose between the effective dose and the toxic dose. Drug carriers, such as those disclosed herein, incorporated as a part of a DDS, may facilitate delivery of a desirable, therapeutic dose to target specific cells all while reducing unintended side effects of regular chemotherapy. Additionally, these carriers may help retain the bioavailability of a drug. Drug delivery systems may be classified into four categories based on their functional mechanism: (1) diffusion-controlled; (2) chemically-controlled; (3) water penetration-controlled; and (4) response-controlled. The latter, response-controlled drug delivery systems are known as “smart DDS” since the mechanism of delivery is based, at least partially, on the sensitivity of the carrier to internal and/or external stimuli in the surrounding environment, such as, but not limited to, temperature, pH, ionic strength, ultrasonic waves, electric field, etc. Using nanoparticles to encapsulate chemotherapeutic agents and to deliver these therapeutic agents to the tumor site may advantageously improve cancer treatment outcomes. Nanoparticles used as carriers in DDS, e.g., DDS for cancer therapy, include at least micelles, nanoemulsions, dendrimers, and liposomes, among others.

Many factors may affect the ability of a DDS nanoparticle to move within and interact with the surrounding environment, including, at least, the nanoparticle's size, shape, charge, coating, cargo, and material.

The size of a nanoparticle may affect the particle's circulation time, extravasation, interstitial diffusion, and the ability to be internalized by the cell. For example, small nanoparticles, e.g., nanoparticles of about 5 nm or less, can escape the bloodstream easily and penetrate adjacent tissues. They are also rapidly filtered by kidneys resulting in a short circulation time that may be as short as only a few minutes. Although small nanoparticles may easily diffuse into tumor tissues, these particles may be repelled by the tumor due to the hydrostatic pressure gradient that is part of the tumor physiology. Larger nanoparticles, e.g., nanoparticles of about 5 nm to 500 nm, may experience improved (e.g., longer) circulation times and may be able to diffuse into the tumor site via pores and/or defects in angiogenic vessels.

The shape of a nanocarrier may contribute (e.g., significantly contributes) to its biodistribution, cellular uptake and the passage through the reticulo-endothelial system (RES), and it may also affect the particle's susceptibility to phagocytosis. Any of these factors may affect the nanocarrier's circulation time. For instance, filamentous micelles, which may have a length of about 18 μm and a diameter of about 20-60 nm, may be retained in circulation for about one week. As another example, PEGylated stealth having a substantially spherical shape may be cleared by the reticuloendothelial system (“RES”) within about two days. Also, the shape of a nanoparticle may affect drug bioavailability inside tumors. For example, worm-like iron oxide nanoparticles may accumulate at higher concentrations inside tumor tissue than nanospherical particles. Moreover, entry and passage of polymeric drug carriers with approximately similar volumes through the pores may be affected by the particle's shape and flexibility. Flexibility and shape may also affect renal clearance of the nanocarrier, thus influencing both the particle's circulation time and biodistribution. When in solution, linear polymers have loose random coil conformations that enable them to easily enter through pores by one end of the chain. Cyclic polymers, which lack chain ends, may need to deform to successfully enter and pass through a pore, thereby making their route into the tissue more difficult.

Liposomes and micelles may be efficient nanocarriers (e.g., they may be the most efficient) for chemotherapeutic delivery. Vesicles with small sizes, e.g., less than about 100 nm, may be able to extravasate from circulation through defects and vascular gaps into tumors due to angiogenesis. The rate of angiogenesis in tumors generally occurs faster than in healthy and/or otherwise normal tissues (and cells). Exceptions to this principle include vessels formed while healing wounds or those formed during pregnancy. Additionally, the retention time of these nanocarriers in tumor sites (e.g., tissues) may be high due to characteristically poor (e.g., low or diminished) lymphatic drainage commonly observed in tumors. Advantageously, the lower size limit of about 20 nm (in diameter) of these carriers (e.g., it may be difficult to form liposomes or micelles having a cross-sectional dimension smaller than about 20 nm) may serve to protect normal cells from being attacked by the chemotherapeutic drug.

While drug delivery vehicles have certain advantages, some factors may limit their efficiency. One limitation that may be observed in connection with some drug delivery vehicles is low bioavailability due to opsonization. Foreign bodies, such as nanocarriers, normally marked for removal by RES organs resulting in their poor (e.g., low or insufficient) accumulation inside tumor tissues. This limitation may be addressed and the circulation time of nanocarriers in vivo enhanced (e.g., improved) by coating them with various polymers, such as, but not necessarily limited to, polyethylene glycol (PEG), which are capable of delaying the nanocarrier's clearance from circulation. Nanocarrier circulation times may be improved by coating (e.g., PEG coating). Increased circulation time combined with low lymphatic drainage and porous angiogenic endothelium commonly observed in tumor tissue contributes to increased nanocarrier accumulation in the tumor tissue due to the enhanced permeability and retention effect (the “EPR” effect). “PEG-lipids” are advantageously water-soluble and biocompatible; consequently, they are safe to use in the body. While PEG coatings may enhance the pharmacokinetics of a drug (e.g., a nanocarrier encapsulated drug) and/or the stability of nanocarriers in circulation, they may reduce tumor uptake because PEG molecules pose a steric barrier between the carrier and the tumor cells.

Nanocarriers that exploit the EPR effect (e.g., depend on leakage into a tumor site followed by the cellular uptake of the drug for accumulation) operate using passive targeting. That is to say, they collect in higher concentrations in tumor tissues merely because of a synergism between the leaky pores of the tumor tissue and the size of the nanocarrier—the nanocarriers are small enough to fit through the larger, leakier tumor pores, but not small enough to diffuse, in any meaningful fashion, through normal vessel walls. By contrast to passive targeting, active targeting uses ligands (molecules that can effectively guide therapies to specific cell biomarkers to achieve certain functionality) or other moieties attached to the surface of nanocarriers to make the drug delivery more selective—active targeting exploits these targeting moieties to improve the co-localization of the drug and cancer cells and/or to improve the cellular internalization via receptor-mediated endocytosis. The ligands attached to the nanocarriers are chosen for their ability to selectively bind to (e.g., affinity for) specific receptors on the surface of the targeted cells.

### Liposomes

Liposomes are defined as a hollow structure made of at least one phospholipid bilayer, similar to the membrane of animal cells. These structures are widely used in drug delivery. Spherical phospholipid structures may be formed when adding water (or another aqueous solution) to a phospholipid film—upon agitation, the phospholipid film forms bilayer spheres that encapsulate small portions of the liquid medium. Liposomes are biocompatible and biodegradable by nature and, due to their amphiphilic nature, they can be loaded with hydrophobic drugs within their hydrophobic lipid bilayer, and with hydrophilic drugs within their aqueous inner core.

Liposomes may be constructed out of phospholipids 300, which are tadpole-like molecules (shown in FIG. 3A) that consist of a hydrophilic head 302 attached to a nonpolar fatty-acid hydrophobic tail 304. The polar head 302 of a phospholipid molecule 300 may consist of glycerol and a modified phosphate group while the nonpolar tail 304 may consist of two long hydrocarbon chains. Phospholipid head groups commonly found in nature generally contain phosphatidylcholine (PC), phosphatidylethanolamine (PE), phosphatidylinositol (PI), and phosphatidylserine (PS). Such phospholipids may be found in soybeans or egg yolks, though neither of these sources is commonly used in human clinical applications due to stability and contamination issues. Synthetic phospholipid derivatives may include, but are not limited to: phosphatidylglycerols, including 1,2 dipalmitoyl-sn-glycero-3-phosphoglycerol (DPPG); phosphatidylcholines, including dipalmitoylphosphatidylcholine (DPPC) and 1,2-distearoyl-sn-glycero-3-phosphocholine (DSPC); hydrogenated soy phosphatidylcholine (HSPC); phosphatidylethanolamines, including 1,2-distearoyl-sn-glycero-3-phosphoethanolamine (DSPE).

Due to the amphiphilic (exhibiting both hydrophobic and hydrophilic behavior) nature of phospholipid molecules in water, they tend to form a bilayer sheet structure, with the phospholipid heads 302 oriented outwards, towards the aqueous medium, and the nonpolar tails 304 being sequestered from contact with the aqueous medium. An example of a bilayer sheet that has formed into a spheroid is shown in FIG. 3B.

Cholesterol 306 may also be present in liposomes and cell membranes (see FIG. 3B). It may be embedded in liposomes to increase their stability by modulating the fluidity of the lipid bilayer and preventing crystallization of the phospholipids acyl chains. When cholesterol 306 is added to unsaturated lipids their permeability to water decreases; hence, liposomes with a high percentage of unsaturated fatty acids may be able to more efficiently hold water-soluble drugs if cholesterol is added. As a result, the addition of cholesterol 306 may permit better encapsulation by the liposome. Also, since cholesterol's steroid rings are dense, their presence increases the mechanical rigidity of the lipid bilayer.

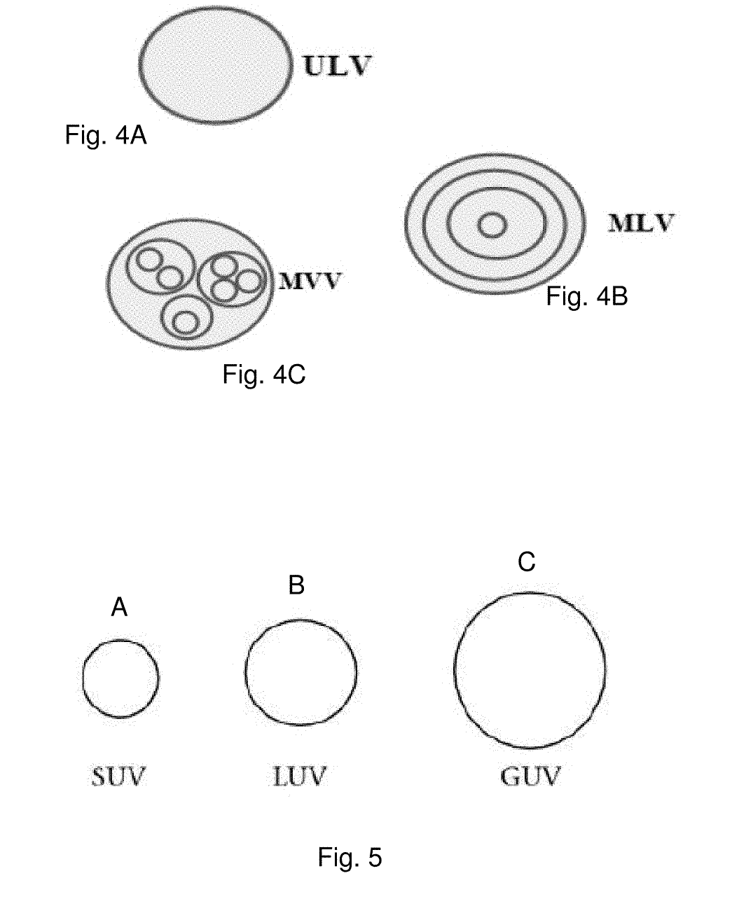

Liposomes may be classified according to their lipid bilayer structures as unilamellar vesicles (ULVs) (an example of which is shown in FIG. 4A), multilamellar vesicles (MLVs) (an example of which is shown in FIG. 4B), and multi-vesicular vesicles (MVVs) (an example of which is shown in FIG. 4C). These groups may be further classified according to their size as small unilamellar vesicles (SUVs) (an example of which is shown in FIG. 5A), giant unilamellar vesicles (GUVs) (an example of which is shown in FIG. 5B), and large unilamellar vesicles (LUVs) (an example of which is shown in FIG. 5C). Additionally, they are classified on the basis of size into small unilamellar vesicles (SUVs) and large unilamellar vesicles (LUVs). Table 2 provides some guidance regarding some possible size ranges for liposomes.

TABLE 2
Different Sizes of Liposomes

| Type of Liposome | Size (in diameter) |
|---|---|
| MVVs | 1.6-10.5 μm |
| MLVs | 0.1-15 μm |
| ULVs | — |
| LUVs | 100 nm-1 μm |
| SUVs | 25-50 nm |

Despite their strengths, liposomes may have some limitations. For example, liposomes may be best suited for encapsulation of hydrophilic drugs, since the incorporation of hydrophobic drugs in their lipid bilayer may disturb their stability. Next, the drug needs to leak from a liposome that has been designed to be stable. Finally, the carried drug cannot be re-encapsulated after release, unlike that which may be observed in micelles, as discussed herein.

### Liposome Preparation Methods

Drug(s) or other therapeutic agents may be loaded into liposomes by either passive or active methods. Variables that may be considered in drug encapsulation are trapping efficiency, drug retention, and drug-to-lipid ratio. Trapping efficiency favors procedures that achieve high drug encapsulation (e.g., greater than 90%). Drug retention is significant for storage purposes and drug release during treatment. Passive loading involves drug entrapment during liposome preparation. Drug and lipids may be dispersed (e.g., concurrently dispersed) in an aqueous buffer. Liposomes will tend to form due to the amphiphilic properties of the dispersed phospholipid. As the bilayer spheres form, they entrap at least some volume of the drug in solution. Active loading involves drug encapsulation after liposome formation. This may be accomplished in a number of ways, including, for example, by establishing a certain membrane potential or transmembrane pH. The choice of the liposome preparation method may depends on one or more of the following factors: (1) the medium used to disperse lipids; (2) the characteristics of the substance to be entrapped and of the constituents used in the liposome formulation; (3) the concentration of the substance to be encapsulated; (4) the desired physical properties of liposomes to be produced such as: size, polydispersity, and the shelf-life of vesicles. Beyond the shape and size classification(s) discussed herein, liposomes may be further classified into three main categories according to their method of preparation: (i) mechanical dispersion, (ii) solvent dispersion, and (iii) detergent removal. Examples of these categories are provided in Table 3.

TABLE 3
Liposome Preparation Methods According to Passive Loading Technique

| Mechanical Dispersion | Solvent Dispersion | Detergent Dispersion |
|---|---|---|
| Methods | Methods | Methods |
| Hand shaken & non-hand | Ethanol or ether | Detergent removal from |
| shaken lipid film | injection | mixed micelles by: |
| hydration, freeze drying | Reverse phase | dialysis, column |
| Sonication | evaporation | chromatography, or |
| Membrane extrusion | Double emulsion | dilution |
| French pressure cells | Stable pluri lamellar | |
| Micro-emulsification | vesicles | |
| Freeze-thawed liposomes | | |
| Dried reconstituted | | |
| vesicles | | |

Liposomes may be prepared according to the lipid film hydration method (one of the “mechanical dispersion methods”) first dissolving lipids in an organic solvent or mixture of organic solvents (e.g., chloroform or chloroform/methanol 2:1 (v/v)), for example in a round bottom flask or vial, to obtain a homogenous mixture with a concentration of about 10-20 mg lipids/ml of solvent. Of course, other concentrations may be used. For example, the concentration of lipids to solvent may be in the range of about 1-100 mg lipids/ml solvent, about 2-90 mg lipids/ml solvent, about 3-80 mg lipids/ml solvent, about 4-70 mg lipids/ml solvent, about 5-60 mg lipids/ml solvent, about 6-50 mg lipids/ml solvent, about 7-40 mg lipids/ml solvent, about 8-30 mg lipids/ml solvent, about 9-25 mg lipids/ml solvent, or any other concentration of lipids to solvent that may advantageously facilitate the formation of liposomes according to any of the various methods disclosed herein. Then, the organic solvent may be evaporated. For example, when the volume of the solvent is comparatively small (e.g., less than about 1 ml), the solvent may be evaporated by purging the sample with a noble gas (e.g., nitrogen or argon). When the volume of the solvent is larger (e.g., greater than about 1 ml), the solvent may be evaporated by using a rotary evaporator. Drying may be performed at a temperature above the phase transition temperature (Tₘ) of the lipids. The lipid film may then be hydrated with an aqueous medium at a temperature above the Tₘ. For example, hydration may be accomplished by placing a round bottom flask containing the lipid film and aqueous medium into a hot bath using a rotary evaporator (without vacuum) for up to one hour or until the film is fully dissolved. If the film is not fully dissolved after one hour more agitation may be advantageous. The aqueous hydration medium may be distilled water, buffered solution, or saline. The product of this synthesis may include a mixture of milky-like multilamellar large vesicles (MLVs).

MLVs produced according to the lipid film hydration method may be reduced to small unilamellar vesicles (SUVs) using various techniques. For example, sonication may be used to mechanically reduce MLVs to SUVs. A sample of MLVs may be sonicated (to reduce the MLVs to SUVs, by either placing a container (e.g., a beaker) containing the sample in a sonicating bath, or by immersing a sonicator probe into a container (e.g., a tube) containing the suspension. Ideally, the sample temperature will not to exceed the Tₘ of the lipids as this may induce de-esterification of the lipids. Therefore, when using a sonicator probe, which delivers a very high energy into the sample and may induce local heating, the temperature of the sample should be carefully controlled. Probe-tip sonicators may also contaminate the sample with metals (e.g., titanium). While metal contaminants may be removed by centrifugation and sample temperatures may be controlled, these issues may be entirely avoided by using bath sonicators; consequently, bath sonicators may be preferable. The temperature of the bath may be held above the Tₘ of the lipids, and sonication may be performed for 10-15 minutes. Of course, other sonication times may be used. For example, sonication may be performed for times in the range of between about 1-30 minutes, between about 2-28 minutes, between about 3-26 minutes, between about 4-24 minutes, between about 5-22 minutes, between about 6-20 minutes, between about 7-18 minutes, between about 8-16 minutes, between about 9-14 minutes, about 10-12 minutes, or any other time that may advantageously facilitate the reduction of MLVs to SUVs according to any of the various methods disclosed herein. During sonication, the suspension will generally convert from a milky to an opalescent solution. Vesicles having small diameters (e.g., less than about 40 nm) produced by/after sonication are metastable. Due to these small diameter vesicles' high curvature energy, they may tend to fuse with other vesicles to form larger vesicles (e.g., vesicles having a diameter of about 60-80 nm), which tend to be more stable.

The reverse phase evaporation (“REV”) method (one of the “solvent dispersion methods”) generally allows for improved (e.g., high) encapsulation efficiency of aqueous medium. Additionally, the REV method may be applied to various lipids, including cholesterol. And, liposomes produced according to the REV method may have an aqueous volume-to-lipids ratio up to 30 times greater than that of SUVs prepared by sonication and four times greater than that of MLVs produced by the lipid film hydration method, discussed herein. The REV method is limited in the number of possible solutions that may be encapsulated: proteins generally may not be encapsulated using the REV method due to the possible denaturation of the proteins upon mixing with an organic medium. And, the encapsulation efficiency that may be achieved using the REV method varies according to the different types and concentrations of lipids used, and is dependent on the ratio of lipids-to-organic solvent-to-buffer.

Liposomes may be produced according to the REV method by first forming inverted micelles by sonication. The synthesis of these inverted micelles begins with a solution of lipids in an organic solvent or mixture of organic solvents (e.g., chloroform or chloroform/methanol 2:1 (v/v)) which is dried (e.g., in a rotary evaporator). Then, the lipids are dissolved in an organic phase, e.g., diethyl ether. Next, an aqueous medium containing the molecules to be encapsulated is added, e.g., in a ratio of about 3:1 (v/v) organic phase-to-aqueous medium, which is required to achieve optimum encapsulation efficiency. While a ratio of 3:1 may advantageously be used, other ratios are possible. To form inverted micelles, the two-phase solution is sonicated for about 2-5 min in a sonicating bath, at a temperature below about 10° C. (to avoid separation of dispersed micelles from the organic phase) until the mixture becomes an opalescent one-phase solution. After sonication, the diethyl ether may be evaporated, e.g., evaporated at room temperature under reduced pressure (e.g., in a rotary evaporator). During evaporation, it is advantageous to avoid foam formation; consequently upon signs of bubble formation the pressure may be advantageously raised to avoid further bubble formation. Following evaporation, inverted micelles become viscous and some of them disintegrate to build up or form a second layer around the remaining inverted micelles thereby forming REV liposomes. Liposomes formed using the REV method are generally unilamellar and may have a heterogeneous size distribution in the range of about 100 nm-1 μm. These REV produced liposomes may be purified by centrifugation or by size-exclusion chromatography (SEC) (e.g., using a Sepharose 4B column).

### Liposome Modification

Liposomes can be modified by varying the liposome lipid composition. For example, liposome formulations containing hydrogenated phosphatidylinositol (HPI) and monosialoganglioside (GM) experience or permit an advantageously high tumor cell drug uptake by comparison to other liposome compositions. Liposomes may be further modified by adding anionic phospholipid, and by changing the amount of cholesterol in the liposome.

Liposomes have many advantages that make them attractive nanocarriers. Some of these advantages include, but are not necessarily limited to: (i) they are generally structurally stable; (ii) they are generally stable in biological fluids (e.g., blood); (iii) they may have a long shelf life; and (iv) their size can be customized. Additionally, liposomes may provide an advantageous encapsulation vehicle for drugs that may beneficially reduce degradation of the drug contained within. Liposomes generally will not extravasate from the bloodstream into normal tissues because these normal tissues possess tight junctions between capillary endothelial cells. As such, liposomes may selectively target tissues and organs with discontinuous endothelia, such as the liver, spleen and bone marrow. Similarly, these nanoparticles may also target tumor tissues due to their similar discontinuous endothelium. The accumulation of liposomes in the tumor site by passive targeting is commonly known as the EPR effect, as discussed herein.

Upon intravenous administration, liposomes are captured by the reticuloendothelial system (RES) and cleared from the blood circulation. The first step in recognizing liposomes is the binding of selected serum proteins to the surface of the liposome (opsonins, such as fibronectin and immunoglobulins), which triggers identification of the “foreign” liposome by the RES. Liposomal capture by the RES may advantageously be exploited in the treatment of RES-related infections, such as leishmaniasis, by delivering antiparasitic and/or antimicrobial drugs using uncoated (non-stealth liposomes). The body's natural clearance system, the RES, serves as a dedicated delivery device for such RES-related infection. However, when liposomes are used to deliver drugs to other tissues, their uptake by macrophages is generally undesirable.

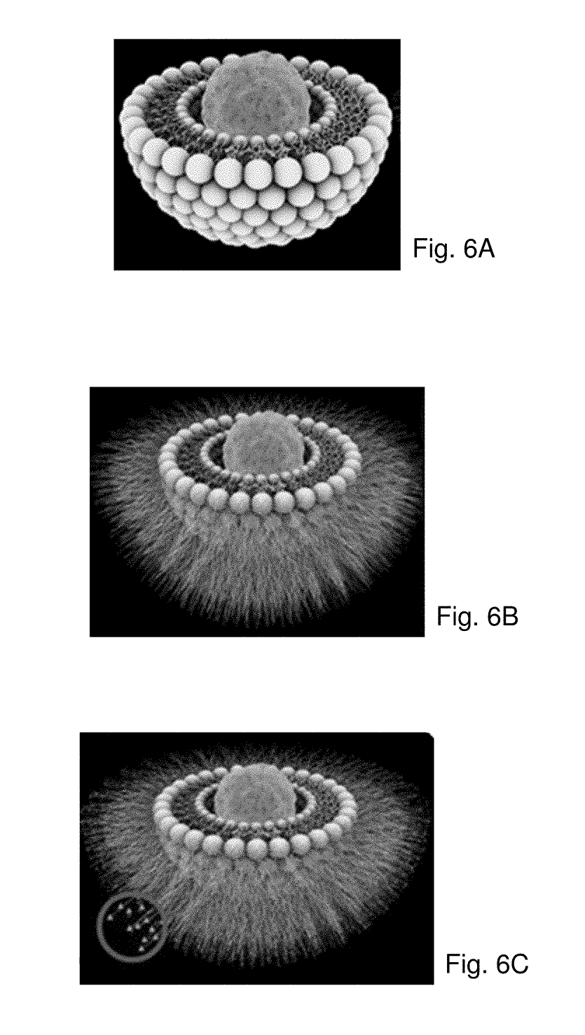

Liposomes may be coated with polyethylene glycol (PEG) or other polymers, which, as discussed herein, may increase the nanocarrier's circulation time. For example, PEG may form a mushroom-like structure or a brush-like structure on the surface of a liposome, depending on its molecular weight and surface density. FIGS. 6A and 6B illustrate the difference between a liposomes with and without PEG coatings: the outermost surface of the nanoparticle in FIG. 6A is formed by the phosopholipids' polar heads whereas, the outermost surface of the nanoparticle in FIG. 6B is formed by branched PEG molecules.

Coating liposomes with hydrophilic polymers, such as PEG, isolates the liposomes from macromolecules such as opsonins and other proteins. Coating liposomes with PEG basically creates a steric stabilization which repels other plasma molecules. Isolation of the liposome may tend to reduce the interaction between liposomes and macrophages and increase the liposome's circulation time. PEGylated liposomes are also known as “stealth liposomes” or “sterically stabilized liposomes” (SLs), as opposed to “conventional liposomes” (CLs) (without a PEG coating). The difference in circulation times between stealth liposomes and conventional liposomes may be significant. For example stealth liposomes may experience a median circulation half-life of 55 hours, whereas conventional liposomes may experience a 6-hour half-life. The use of PEG has many advantages. For example: PEG is safe, non-toxic, biocompatible, and soluble in aqueous medium. Moreover, PEG has already been approved by the FDA for at least some uses. As a result, PEG coating may prolong the circulation half-life of the nanovehicles in the body, and decrease their degradation by metabolic enzymes.

Modification of the liposomal surface with PEG (sometimes known as PEGylation) can be achieved in many ways. For example, through physical adsorption, by incorporating PEG-lipid conjugate during liposome preparation, or by covalently attaching reactive groups to the surface of the liposomes. Poly(ethylene) glycol not only has the potential to reduce uptake of liposomes by macrophages, but it may also enhance the stability of liposomes by reducing aggregation. For liposomes composed of phospholipids and cholesterol, the ability to increase the circulation time by PEGylation depends at least in part (e.g., mainly), on the amount of grafted PEG and the length, or equivalently, the molecular weight of the polymer. For example, long chains of PEG attached to a liposome's surface may increase the circulation time by comparison to an uncoated liposome. It may be advantageous to reversibly attach PEG to the liposomal surface in order to improve the continuous capture of liposomes by the cells. For example, a lower pH (e.g., the lower pH commonly found at tumor sites) may be exploited to detach the PEG coating once liposomes accumulate at the site of the lower pH (e.g., in the tumor).

A potential limitation of both conventional and stealth liposomes may be observed after the first dose of treatment. A second dose of stealth liposomes, injected a few days after the first dose, may experience a decreased circulation time and despite the presence of PEG, instead of circulating, the liposomes may accumulate in the liver. This “accelerated blood clearance (ABC)” may be initiated by the production of anti-PEG Immunoglobulin M (IgM) in the spleen in response to the previous dose of stealth liposomes. Anti-PEG IgM binds to PEG and activates the complement system to opsonize liposomes. Once opsonized, the PEGylated liposomes may be captured by Kupffer cells in liver. Accelerated blood clearance may also be observed when the first dose of liposomes comprises conventional liposomes (instead of stealth liposomes) followed by a second/later dose of stealth liposomes (not including conventional liposomes). Certain effects of accelerated blood clearance may be correlated to factors such as the type and the amount of the initial dose, the interval between injections, the surface density of grafted PEG, and the surface charge of the nanocarrier (e.g., liposome). Indeed, accelerated blood clearance may be inversely related to the initial amount of the PEGylated liposomes administered. Moreover, accelerated blood clearance may occur only slightly occurred after a first dose of conventional liposomes (i.e., without PEG) having a size of about 110 nm, regardless of the charge of liposomes. And, administration of smaller liposomes (e.g., about 60 nm) may cause increasingly accelerated blood clearance.

As is discussed further herein, liposomes can be further modified to selectively increase drug accumulation in a desired location. So-called “targeted liposomes” may be prepared by conjugating moieties to the surface of the liposome, allowing them to recognize and bind to specific receptors (e.g., specific receptors on the surface of cancer cells). Examples of molecules that can be used as ligands or targeting moieties include, but are not limited to: antibodies and their fragments, lectins, albumin and other proteins, lipoproteins, hormones, charged molecules, monosaccharides, oligosaccharides, polysaccharides, and some low-molecular-weight ligands (of which folic acid may be the most common). One advantage of using targeted liposomes instead of plain liposomes includes an increase (e.g., a dramatic increase) in the amount of drug delivered to the target site and the so-called “bystander killing” effect, which is caused by the diffusion of the drug molecules to adjoining tumor cells.

### Micelles

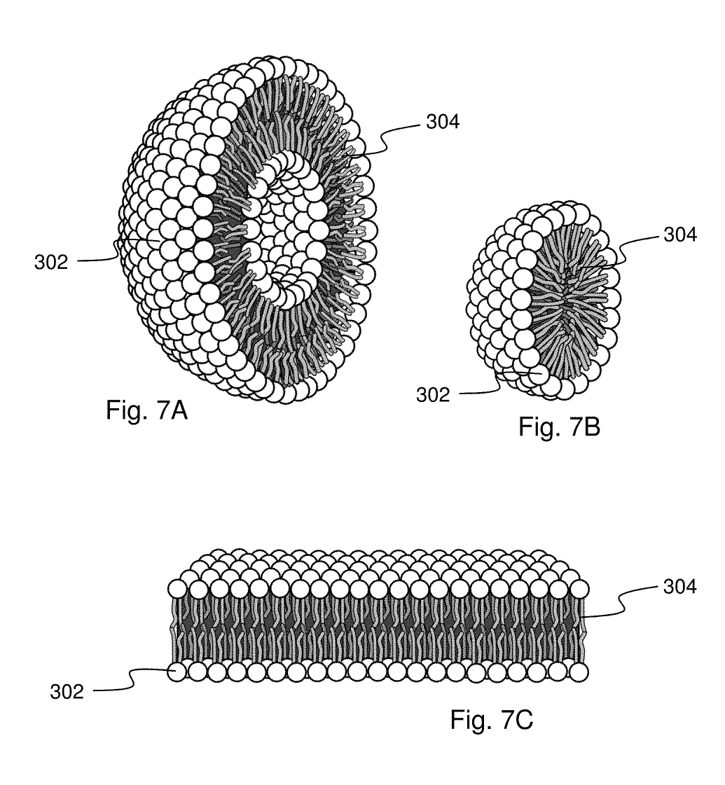

Micelles are spherical nanoparticles with a diameter ranging from about 5-100 nm that result from the self-assembly of amphiphilic molecules (these amphiphilic molecules may be the same basic building blocks as those used in liposomes) in an aqueous solution, originating a structure with a hydrophilic or polar corona and a hydrophobic core. The structures of a liposome, micelle, and lipid bilayer are compared in FIGS. 7A, 7B, and 7C, respectively. Each of these figures shows the location of the polar heads 302 and the hydrophobic tails 304. Hydrophobic drugs can penetrate and accumulate inside the hydrophobic center which decreases the interaction with the aqueous exterior. Advantages of micelles include their easy preparation methods (usually just dissolving the copolymers in an aqueous solution), simple drug loading (just by mixing the micelles with the drug), and their stability and controllability. However, when injected intravenously, some micellar formulations may suffer a rapid clearance by the mononuclear phagocyte system. To remedy such rapid clearance, micelles may be modified by coating them with agents, such as PEG (discussed elsewhere herein).

The most structurally stable micelles are polymeric micelles, which are used in most DDS. These micelles are diblock, triblock, or even more complex structures of copolymers. Pluronic® micelles, composed of poly(ethylene) oxide and poly(propylene) oxide blocks, have been widely studied as nanocarriers. They may be particularly useful in acoustically activated drug delivery because at least one of their members, Pluronic® P105, has been extensively studied to deliver Dox in conjugation with ultrasound. Polymeric micelles may be formed even at very low polymeric concentration; hence, they are generally stable due to their slow dissociation, which ranges from hours to days. Additionally, their size generally allows them to extravasate at the tumor site, due to the enhanced permeability and retention (EPR) effect (as discussed herein in connection with liposomes and other nanocarriers), while escaping renal excretion.

### Dendrimers

Dendrimers are three-dimensional, synthetic, highly branched, polymeric macromolecules. They are generally spherical in shape with layers of branches around the core. Dendrimers may have attractive properties to drug delivery applications. For example, they are generally water soluble, have uniform size, include inner cavities, and their surfaces can be modified according to their functions. In addition, their biocompatibility, polyvalence and precise molecular weight can make them ideal nanocarriers for drug delivery applications. Dendrimers can carry both hydrophilic and hydrophobic drugs. Common types of dendrimers include poly(propylene imine) (PPI), polylysinedendrimers, poly(amidoamine) (PAMAM). The dendrimer PAMAM may be transported through bio-membranes by paracellular and endocytosis processes.

### Nanoemulsions

Emulsions can be defined as a mixture of two insoluble liquids, one of which is the continuous phase while the other liquid is the dispersed phase. The simplest example of an emulsion is oil-in-water. The droplet diameter ranges from 50 to 1000 nm. In drug delivery, the continuous phase is usually aqueous and the dispersed phase is commonly where a hydrophobic drug is carried.

Nanoemulsions may be particularly attractive because nanoemulsions have small droplet sizes, which may allow easier extravasation when releasing drug and can cause large reduction in gravity, and no sedimentation occurs on storage. In some applications, nanoemulsions may be used as a substitute for liposomes.

### Protein-Bound Paclitaxel

Protein-bound paclitaxel is a nanoparticle that carries paclitaxel into cells (e.g., cancer cells). It may be used to treat breast, lung, prostate and other cancers. Although conventional paclitaxel has also been used in the treatment of cancer, it has some drawbacks, such as its reliance on solvent-based delivery carriers injected intravenously, which can cause serious toxicity. Thus, a nanocarrier protein-engineered therapy is advantageous. Using protein-bound paclitaxel nanocarriers, one can avoid the need for premedication and prolonged administration time. It can also increase the ease of administration. The first approved protein bound paclitaxel by FDA is albumin-bound paclitaxel called Nab™-paclitaxel, which is administrated over a short period of time and does not need intravenous tubing.

### Targeted Drug Delivery Systems

An active pharmaceutical ingredient (API), when administrated to a patient, is distributed within the body proportionally to blood flow. Furthermore, along its journey to the site of action, the API has to cross many organs, tissues and cells, where it can be deactivated. Otherwise, it can lead to undesirable effects for sites that are not meant to be reached. Thus, to ensure that the drug reaches the target site, it is frequently administrated in large amounts. This may cause negative effects on healthy cells, especially if the drug is cytotoxic. In the best case, the drug is wasted in normal tissues causing an increase in therapy cost. A solution to such problems can be introduced using drug targeting. Drug targeting can be defined as an increase in the selective and quantitative accumulation of an API in a certain tissue or organ in the body, thereby reducing side effects of unspecific drug accumulation. The interaction of four main components makes the targeted delivery possible: chemotherapeutic ingredient, targeting moiety, carrier (e.g., nanoparticle), and a target.

Nanoparticles can also be designed to release drug(s) by external stimuli such as magnetic fields or US, which is a process called triggered targeting. Other nanoparticles may be sensitive to changes in pH or temperature which can be used to administer a drug into a target area with a different pH and/or temperature.

Calcein, a fluorescent dye, may be used as a model drug to simulate the use of the chemotherapeutic ingredient. Calcein advantageously allows the characterization of release profiles due to the ease by which fluorescence may be measured. However, it should be understood that any of a number of chemotherapeutic agents, or other drugs may be used instead of calcein to achieve a desired therapeutic outcome. In some embodiments, human serum albumin (HSA) is used as the targeting moiety, and liposomes as carriers. In some embodiments, 20-kHz low frequency US (LFUS) is used as trigger to release calcein encapsulated in the prepared liposomes.

### Passive Targeting

The vasculature of tumor tissues differs from that of healthy tissues in its functionality and morphology. In tumors, the blood vessels frequently have defective vaso-architecture, including irregular shapes, and lack of a smooth muscle layer. The endothelial cells are also not well organized. Furthermore, they are dilated, wide and leaky. The basement membrane in the tumor is usually abnormal or even absent in some cases. Additionally, tumors frequently have impaired lymphatic drainage of macromolecules and lipids.

These characteristics of tumor tissues lead to the enhanced permeability and retention (EPR) effect that allows extravasation of drug-loaded nanoparticles in tumor cells. The EPR effect can be observed with molecules that have long plasma half-lives, with an apparent size higher than 50 kDa, above the kidney clearance threshold (5 nm). Moreover, the molecule should be neutral or anionic but preferably not cationic, because the inner surface of blood vessels is highly negatively charged. The EPR effect is usually characterized by imaging tumor blood volume and flow. When the delivery essentially only relies on the pathophysiological properties of the target cancer tissue, as in non-ligand nanoparticles, it is commonly referred to as the passive drug targeting.

One of the most meaningful examples of passive targeting carriers approved for clinical use is Doxil®. It is an echogenic PEGylated liposome loaded with Dox and used in the treatment of many cancer types, including ovarian cancer, breast cancer, and hormone-refractory prostate cancer (HRPC). Other examples include Myocet (non-PEGylated liposome-Dox) and Daunoxome (non-PEGylated liposomal daunorubicin). Even with PEGylated liposomes, generally less than 5% of the administered drug accumulates in tumor cells by passive targeting.

While most of the drugs have a plasma half-life of 20 minutes in humans and mice, it takes around 6 hours of circulation for any drug to exert the EPR effect. Thus, DDS exploiting the EPR effect may be improved by using the stealth liposomes discussed herein, and other modified liposomes.

### Active Targeting

Active targeting is a type of targeting achieved by conjugating a carrier with another molecule (called a ligand or targeting moiety) to actively deliver the drug to tumor cells. It is particularly useful when passive targeting is not possible or sufficient, which may occur when vascular permeability, pH, and/or temperature of the affected area do not significantly differ from that of normal tissues. Also, active targeting may be employed in targeting within the circulatory system. One advantage of active targeting is its ability to effectively deliver high loads of drugs while minimizing and/or avoiding MDR.

The simplest approach to prepare targeted drugs includes direct coupling of a drug to a targeting moiety. One common example of such an approach includes immunotoxins, where a natural toxin is split into active and recognizing moieties. The active moiety is the toxic part that is conjugated with an antibody. Another approach includes the use of a nanocarrier, e.g., liposomes, which can be modified by conjugation with a targeting moiety. The targeting moiety or ligand can recognize certain binding sites on the tumor cell surface, so that the carrier remains attached to that surface, and ready to release its drug load upon request.

One embodiment of targeted nanocarriers disclosed herein is PEGylated liposomes with their surface modified by covalently bound albumin. Human serum albumin (HSA) is a naturally occurring protein in human plasma that is produced in the liver. Human serum albumin may be used as a drug carrier molecule by itself, such as albumin-bound paclitaxel. Albumin is a natural carrier of hydrophobic molecules. Another example of albumin being used as a nanocarrier is Dox loaded on HSA nanoparticles by at least one of two methods: adsorption and incorporation into the particle matrix. Both strategies can result in nanoparticles with a size range between about 150-500 nm and loading efficiency as high as about 95%. Other methods may be used to prepare PEGylated-liposomes targeted with albumin (PEG-L-A).

Cancer cells frequently overexpress one or more receptors (e.g., certain receptors have been identified as being overexpressed in certain types of cancer) such as epidermal growth factor, folate, transferrin, fucose, and/or estrogen. To use liposomes in active targeting, targeting moieties may be attached to the surface of liposomes. An example of a PEGylated liposome to which targeting moieties have been attached is shown in FIG. 6C. Targeting moieties, such as monoclonal antibodies (MAb), fragments of proteins, peptides, carbohydrates and other receptor ligands, may allow specific targeting for certain receptors on the surface of cells. Targeting during drug delivery may advantageously allow delivery of the cytotoxic agent to specific tissues, thereby reducing potentially adverse side effects of conventional chemotherapy. A large number of ligands can be attached to the liposomal surface allowing for multivalency (or multispecificity) that may achieve high avidities using ligands of comparatively low individual affinities.

Ligands may increase uptake of conjugated/functionalized liposomes by the liver and/or spleen, even in the presence of PEG (e.g., even when the functionalized liposome is also PEGylated). Therefore, a lower surface density of ligands may advantageously allow binding of the liposome to the desired target, while maintaining a prolonged circulation (e.g., avoiding clearance by the spleen and/or liver). Ligands used in active targeted drug delivery may be classified as exogenous ligands, which initiate immunological reactions, or endogenous ligands, which are non-immunogenic and tend to be biocompatible. Endogenous ligands include antibodies, polypeptides, hormones and fusogenic proteins. Some of the receptors binding to endogenous ligands, such as transferrin receptors, lipoprotein receptors and hormone receptors, are overexpressed in cancer cells as compared to normal cells.

Antibodies are ligands that may be attached to liposomes' surfaces to create immunoliposomes. When used as ligands, antibodies may have certain advantages over peptides. For example, antibodies have high affinity and specificity for a wide range of antigenic determinants, maintain a uniform structure, are generally biocompatible, and have well-established chemistry.

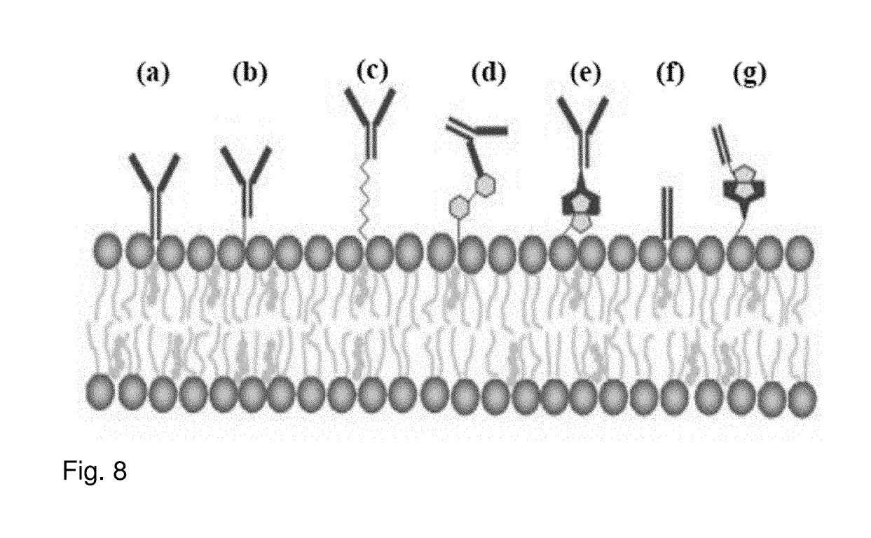

There are different ways to bind ligands to liposomal surfaces, e.g., surface adsorption and covalent coupling. FIGS. 8A-8G illustrate examples of functionalization methods, including: surface adsorption of the targeting moiety as shown in FIG. 8A; covalent coupling of the targeting moiety as shown in FIG. 8B; PEG spacers attached to the targeting moiety (e.g., the targeting moiety may be attached to the PEG of a PEGylated liposome, as disclosed herein) as shown in FIG. 8C; hapten binding to the targeting moiety as shown in FIG. 8D; and avidin-biotin attached to the targeting moiety with either avidin or biotin attached to the surface as shown in FIGS. 8E & 8G. The location where the ligands are attached may be critical. For example, antibodies bound directly to the liposome surface adjacent to PEG may exhibit less effective targeting capabilities than those attached to PEG terminal ends. It is likely that this is due merely to the steric interference provided by the co-attached PEG.

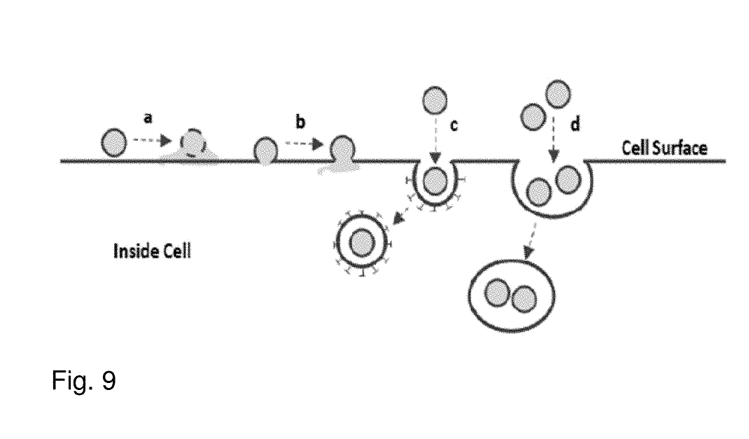

After attaching a targeting moiety, a biological mechanism is needed to deliver the nanocarrier and encapsulated drug to/into the tumor site. Liposomes can deliver their content to cells through various mechanisms including: extracellular release as shown in FIG. 9A; membrane fusion as shown in FIG. 9B, endocytosis (e.g., receptor-mediated endocytosis) as shown in FIG. 9C, and phagocytosis as shown in FIG. 9D. As shown in FIG. 9A, liposomes may release their content after surface absorption, so that free drug is absorbed by the cell. As shown in FIG. 9B, liposomes may fuse with the cell membrane to deliver their content. As shown in FIG. 9C, liposomes less than 150 nm in diameter may attach to the cell surface receptors, and then be drawn into clathrin-coated pits to form coated vesicles. These vesicles diminish later and liposomes are fused with lysosomes where lipids are degraded and the encapsulated drug is released. As shown in FIG. 9D, phagocytosis (pino-cytosis) may occur to engulf molecules larger than 150 nm.

Some ligands are classified as endogenous ligands (also known as bio-self molecules) may be advantageously non-immunogenic and biocompatible. These ligands include, but are not limited to, antibodies, polypeptides, hormones, and fusogenic proteins. Some of the cellular receptors that bind to endogenous ligands include, but are not limited to, transferrin receptors, lipoprotein receptors, hormone receptors, and certain receptors that may be present on tumor cells.

Estrogens are sex hormones that play a crucial role in the development of reproductive organs. They are also steroid hormones that work as chemical messengers in the body. Endogenous steroidal estrogens include estrone, estradiol, and estriol, which are produced (primarily in the ovaries and secondarily in adrenal cortex, testes, and placenta) from testosterone and androstenedione. Each form of estrogen plays a dominant role at a different point in a woman's lifetime: estradiol is the main form of estrogen produced during reproductive years; estriol is the main estrogen produced during pregnancy, and estrone is the main estrogen produced after the reproductive years, i.e., while in and after menopause. Estradiol is the most potent estrogen due to its high affinity in receptor-ligand interaction. Estrogens also play an important role in the regulation of the urinary tract, bones, heart and blood vessels, breast, skin, and hair. In the reproductive system, estrogens support the development of female breast tissues, while in the cardiovascular system, they play a role in lowering the concentration of lipids that affect blood vessels, and also increase the blood flow in vessels and decrease the vascular resistance. Estrogens are also biologically produced in the brain and body fat, therefore, they may interfere with functions of the nervous system such as memory, temperature regulation, and/or awareness.

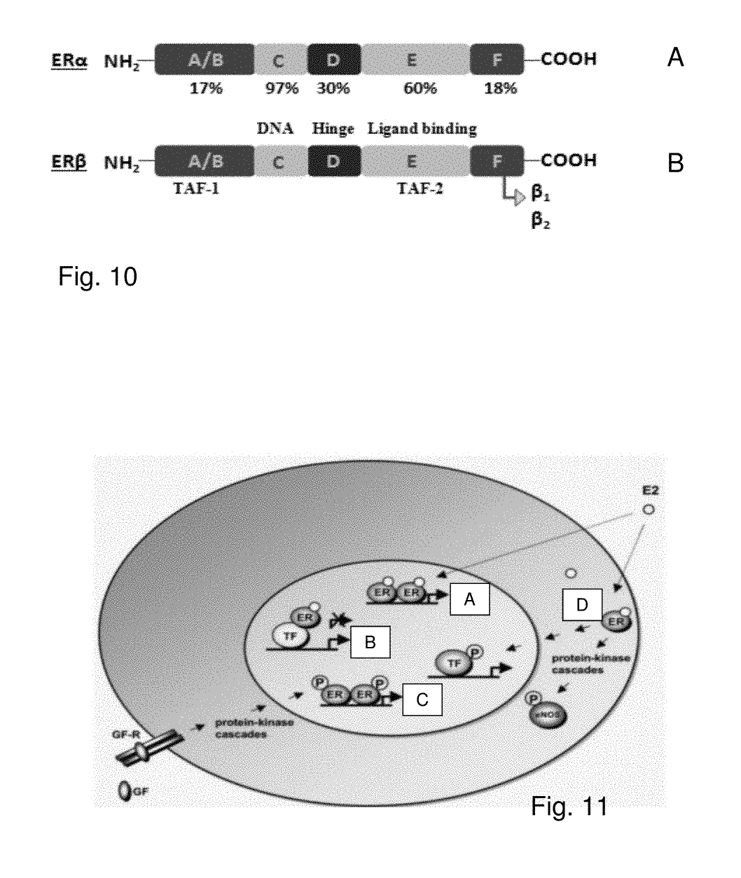

The function of estrogen is mainly mediated by two receptor proteins: estrogen receptor alpha (ERα) and estrogen receptor beta (ERβ). Estrogen receptors (ERs) consist of a single polypeptide chain that contains six functional domains, shown in FIG. 10: FIG. 10A shows the structure of ERα while FIG. 10B shows the structure of ERβ. The molecular weights for human ERα and ERβ are 66 kDa and 54 kDa, respectively. The percentage of homology between each of the functional groups in ERα and ERβ is shown in FIGS. 10-10B: domain A/B has 17% homology; domain C has 97% homology; Domain D has 30% homology; Domain E has 60% homology; and Domain F has 18% homology. Domain C, which shows the highest percentage of homology (e.g., the highest degree of similarity) between the two types of receptors, includes two zinc fingers that bind hormone elements responsible for DNA interactions. Domain A/B, the N-terminal, shows the least percentage of homology (e.g., the lowest degree of similarity) between the two types of receptors, and includes the ligand-independent-transcriptional-activation function TAF-1. The D-domain is a hinge region. And, the E-domain includes the hormone binding domain and the hormone-dependent-transcriptional-activation function TAF-2.

Estrogen receptors are expressed in organs/systems other than the reproductive system, including, but not limited to, the liver, cardiovascular system, bones, and breasts. ERα is generally overexpressed in breast cancer cells and the stromal compartment of both prostate and ovarian stroma cells. Thus, estrogen ligands may be advantageously used in drug delivery to target cancer cells via estrogen ligand-receptor interaction. ERβ is expressed in the central nervous, cardiovascular, immune, and reproductive systems. Steroid hormones are lipophilic, which allows them to pass through the cellular membrane by passive diffusion and bind to their corresponding receptors that are located either in the nucleus or in the cytoplasm and sometimes on the exterior membrane of the cell.

Estrogen-receptor binding interaction may occur by several mechanisms. For example, the mechanism may be any of the following: the classical mechanism of ER action (shown in FIG. 11A); ER-independent genomic mechanism (shown in FIG. 11B); ligand-independent genomic mechanism (shown in FIG. 11C); and non-genomic action (shown in FIG. 11D). The classical mechanism involves the direct binding of estrogen to its corresponding receptor in the nucleus. This causes the receptor to dimerize and bind to estrogen response elements (EREs) that are located on the target genes. However, about one-third of the human genes that are regulated by estrogen receptors lack the ERE sequence, suggesting that additional mechanisms are needed to regulate expression of these genes. Second, the ER-independent genomic mechanism controls the gene expression by regulating transcription factors through protein-protein interaction instead of directly binding to DNA. This mechanism may be used to regulate genes lacking the ERE sequence. Third, the ligand-independent genomic mechanism includes growth factors (GFs) that activate the protein-kinase cascades. This leads to the phosphorylation (P) and subsequent activation of ERs at the ERE sequences on the genes. The last mechanism is characterized as non-genomic and may occur faster than those previously discussed (i.e., the genomic mechanisms). In this mechanism, the estrogen-ER complex phosphorylation (P) targets proteins, which activates their function in the cytoplasm or triggers the action of transcription factors (TF) in the nucleus.

At least some types of cancer, including, but not necessarily limited to breast cancer, are dependent on hormones, such as estrogens. Estrogen may have two different roles that may explain its impact on breast cancer development. The first is the proliferation of mammary cells after estrogen binds to its receptor, which results in an increase in cell division and DNA replication that ultimately leads to lethal mutation. The second is a point mutation resulting from DNA damage caused by side products of estrogen metabolism. In both cases, there is a disturbance of apoptosis and DNA-repair mechanisms, leading to tumor development.

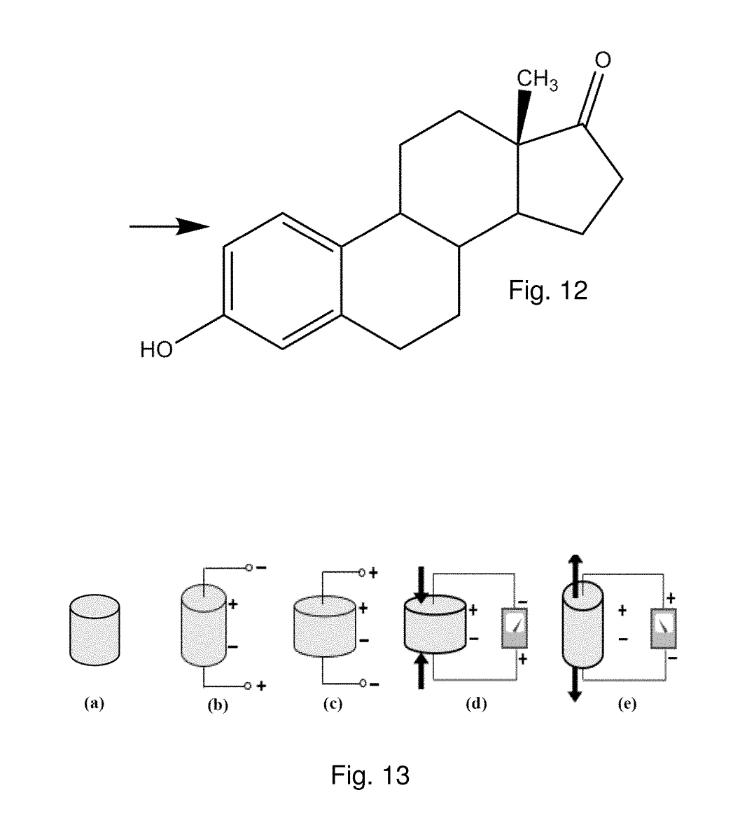

Estrogen receptors are present in two-thirds of breast cancer and are distributed in the nucleus and on the cell membrane. The presence of ERs on the cell membrane can be used to guide therapies towards breast cancer cells, e.g., as a targeting mechanism. In postmenopausal women, the major estrogen is estrone, which is primarily produced from the aromatization of androstenedione, which is mainly (e.g., ˜95%) produced in the adrenal glands. Estrone has the second highest affinity to ER after estradiol. It transfers the majority of its signal (e.g., about 60%) to ERα while the remainder of its signal is transferred to ERβ (e.g., about 37%). Since breast cancer overexpresses ERα, estrone may be a potential ligand (or targeting moiety) for use with cancer therapeutic drugs, especially for postmenopausal women. Estrone has a molecular weight of 270.4 g/mol and its chemical formula is C₁₈H₂₂O₂ with a chemical name of 3-hydroxyestra-1,3,5(10)-triene-17-one. FIG. 12 illustrates a 2-D molecular representation of estrone. As a result, estrone liposomes (as discussed herein) could avert opsonization, allowing for a longer circulation time of the carrier in the blood and maximizing the cell-specific delivery of the therapeutic

Cellular uptake of drugs may be enhanced by actively-targeted nanoparticles, such as estrone. However, other active-targeting moieties may be used. For example, cellular uptake of liposomal apatinib may be enhanced by the conjugation of cyclic-RGD to liposomes. Additionally, functionalizing liposomes using SP5-2 may improve cellular uptake of the liposomal contents. In breast cancer cells, transferrin-modified nanoparticles may exhibit markedly increased uptake when compared to control liposomes. Similarly, several other actively targeted therapies may be used to address various cancers, including therapies functionalized with one or more of the following targeting ligands: transferrin, estrogen, tamoxifen, folic acid, and RGD.

### Ultrasound

After a nanocarrier (e.g., a passively targeted nanocarrier, such as liposomes accumulated due to the EPR effect, or an actively targeted nanocarrier, such as estrone functionalized targeted liposomes) reaches a cancer site, a stimulus may be used to trigger release of an encapsulated drug. The use of external triggers, such as electric or magnetic fields, pH or temperature changes, etc. is known as actuated targeting. Ultrasound may be used as a trigger mechanism in actuated targeting systems, e.g., for US-sensitive liposomes, called echogenic or acoustically active liposomes (AAL).

Ultrasound is composed of oscillatory pressure waves that may have frequencies higher than the audible limit of humans (e.g., >20 kHz). Pressure waves, also known as stress waves, require a medium to propagate because their transmission occurs by direct contact of masses. These waves depend on the elastic nature of the medium, which plays a key role in sustained vibrations (thus, stress waves are sometimes called elastic waves). Pressure waves can be induced by piezoelectric transducers (e.g., vibrating piezoelectric transducers).

Ultrasound waves can be classified, according to their intensities, into low-intensity and high-intensity waves, which have different applications. For example, applications like nondestructive characterization of materials, medical diagnosis, and the area of sensors, which only require the transmission of energy through a medium without changing it, may use low-intensity US. When US waves are meant to cause/impose an effect on the medium being propagated through, the suitable choice is to use waves with high intensity. Ultrasound waves with high intensity are often associated with thermal or mechanical effects which can be used to induce cavitation events. These may be applied, for example, in kidney stone shattering, tumor ablation, cell lysis, emulsification, atomization of liquids, and/or welding of materials (e.g., plastics, metals, etc.). US may also be used as a triggering mechanism for DDS employing various types of nanocarriers by inducing mechanical and/or thermal effects on the nanocarriers.

### Generation of Ultrasound

Ultrasound waves can be generated in at least three ways, including the Galton's whistle method, the magnetostriction method, and the piezoelectric method. Galton's whistle uses a special type of whistle to generate US waves that can be used to train animals. Such whistles are capable of producing sound waves with frequencies up to about 30 kHz. Magnetostriction involves a change in at least one dimension (e.g., a change in the dimensions) of a ferromagnetic material (e.g., iron, nickel, etc.) having a rectangular-bar shape when a magnetic field is applied along its axis. Application of a non-oscillating magnetic field generally results in a minor increase in the bar length (e.g., about 10⁻⁶ of the original length for a nickel bar). Application of an oscillating field generally causes a significant increase in the bar length since the elasticity of the material can no longer counteract the change imposed. Magnetostriction may be used to generate US with a maximum frequency of about 2 MHz. Specific crystals (e.g., piezoelectric crystals), like quartz, rochelle and tourmaline salt, accumulate electric charge on their surface upon exposure to a mechanical pressure/tension. In a reverse manner, a piezoelectric material vibrates when an electric charge is applied to its surface. When an electric field (e.g., voltage difference) is applied to a cylindrical ceramic bar (e.g., after polarization of the bar) in the same direction of the poling voltage, the bar will undergo elongation/lengthening (e.g., expansion). When the voltage direction is reversed, the bar will undergo shortening (e.g., compression). In a piezoelectric material, mechanical stress (e.g., tension/compression) may be translated into electrical energy, as shown in FIG. 13. FIG. 13A shows a piezoelectric bar that is polarized with positive located at the top of the bar and negative located at the bottom of the bar. FIG. 13B illustrates the piezoelectric bar with a voltage applied reverse to the polarization of the bar—negative at the top and positive at the bottom. As can be seen, reverse application of voltage causes the bar to lengthen with respect to its resting (natural) configuration. FIG. 13C illustrates the piezoelectric bar with a voltage applied in the same direction as the polarization of the bar—positive at the top and negative at the bottom. As can be seen, voltage application in the same direction causes the bar to shorten with respect to its resting (natural) configuration. FIG. 13D illustrates a measure of the voltage drop across the piezoelectric bar when the bar is under compression. As can be seen, when the bar is compressed, a voltage is generated (e.g., a positive voltage is generated). FIG. 13E illustrates a measure of the voltage drop across the piezoelectric bar when the bar is under tension. As can be seen, when the bar is stretched, a voltage is generated (e.g., a negative voltage is generated).

Generally, the main components required to construct a device that produces US waves via piezoelectric compression and expansion, include a transducer, a pulse generator, and an amplifier. The transducer contains the piezoelectric material that translates electric pulses into mechanical vibrations. In medical scanning devices, a transducer may also operate in the reverse direction, receiving echoes (mechanical waves) and generating electric signals as a characteristic of the scanned medium. The pulse generator generates regular electric pulses (to be applied to the transducer) and may allow the user to control the pulse frequency and amplitude, among other features. Finally, amplifiers may be used in circuits to magnify the magnitude of an electric signal. Other accessories may be added to the circuit depending on the application.

### Physical Properties of Ultrasound

The energy of US waves propagates through a medium by collisions of oscillatory particles but with no net displacement. These waves can be focused, reflected, and/or refracted. Ultrasound waves are sinusoidal waves with a given frequency. When two ultrasound waves of different frequencies interfere, their amplitudes are added/subtracted and the resulting frequency is the result of the individual frequencies of each wave. Consequently, the superposition of ultrasound waves can cause beats; hence a wave is known to have a “beat frequency” that may be coupled with the Doppler Effect in many applications. Doppler Effect is the change of the wave frequency to a moving observer as the observer moves relative to the wave source. Once an ultrasound wave propagates into a medium, its amplitude diminishes (e.g., attenuated). Attenuation occurs due to several factors including absorption of waves (e.g., resulting from the conversion of energy, such as the conversion of mechanical energy into heat) and reflection and scattering of waves by irregular surfaces/interfaces. The intensity of an ultrasound beam may be selected for a specific application. High-intensity US may generate intense heat, e.g., sufficient to melt steel. High-intensity US (e.g., when applied to liquids) may cause cavitation—cavitation may be exploited in drug delivery (e.g., DDS using acoustically activated targeted liposomes) or in various cleaning processes.

Ultrasound waves may be characterized by their frequency, propagation speed and amplitude. When an ultrasound wave propagates from one medium to another, both amplitude and velocity are affected, but the shape of the wave remains unchanged. The velocity of a wave depends on the nature of the medium (its density and elasticity) and the type of the wave, while its amplitude depends on the impedance ratio of both mediums. Additionally, there are several modes of US vibration that can be utilized in ultrasonic applications, including: longitudinal, transverse, torsion, shear, surface, flexural and Rayleigh. Longitudinal waves (also known as compressional waves) are characterized by molecules vibrating in parallel to the direction of energy transfer. Transverse waves are characterized by molecular vibrations that are orthogonal to the direction of energy transfer. Different types of ultrasound waves may be limited or restricted based on several factors. For example: transverse waves can only propagate through a solid medium; gases can only transfer longitudinal waves; liquids can transfer both longitudinal and surface waves. Longitudinal waves may be advantageously applied to certain biological-interaction systems because of the potentially favorable sequence of compressions and rarefactions that may be created by these waves.

### Acoustic Impedance

When an acoustic wave propagates through a fluid, the particles of that medium are forced to displace around their original position with a velocity known as acoustic particle velocity. However, in any medium, there is resistance to acoustic wave propagation, which is called acoustic impedance (with SI units of Pa·s/m³). Acoustic impedance affects the proportion of acoustic energy transmitted and/or reflected. When a medium is characterized by closely-packed particles (i.e., a dense material, high specific acoustic impedance), the particles require high pressure to move at a given velocity compared to a lower pressure required for loosely-packed materials (i.e., low specific acoustic impedance) at the same velocity. Equation 1 relates the pressure of an acoustic wave (P), the speed of sound in the medium (c), the particle velocity (v) and the density of the medium (p) for which the wave is propagating through:

P=ρ·c·v=Z·v Eq (1)

Based on Equation 1, the specific acoustic impedance (Z) (Pa·s/m, equivalent to Rayl) for a substance depends, at least partially, on the density of the substance and the velocity of the acoustic wave. Table 4 lists the specific acoustic impedances for some materials and tissues.

TABLE 4
Characteristic acoustic impedance for selected biological tissues

| | Characteristic Acoustic Impedance |
|---|---|
| Tissue | (Rayls) |
| Water (20° C.) | 1.48 × 10⁶ |
| Muscle | 1.65-1.74 × 10⁶ |
| Fat | 1.38 × 10⁶ |
| Skin | 1.7 × 10⁶ |
| Cortical bone | 4-8 × 10⁶ |

### Reflection of Waves

When a pressure wave (e.g., an ultrasound wave) strikes a boundary (e.g., a bone-tissue interface), the characteristic acoustic impedance of both media determines the fraction of the wave's energy that is reflected as an echo. The difference between two medias' impedances is known as an acoustic impedance mismatch (the mismatch factor). The greater the difference between the impedances, the more the acoustic energy is reflected from the interface and, concomitantly, the less energy is transmitted into the second medium. If the impedances of both media are identical, there will be a complete transmission with no reflection. The angle of wave incidence also affects the proportion of energy that is reflected and transmitted from and through the interface. When a beam strikes a surface at an orthogonal angle, the fraction of the reflected (R) and transmitted (T) energies for longitudinal waves is represented by the following,

R=(Z₁−Z₂)²/(Z₁+Z₂)² Eq. (2)

T=4Z₁Z₂/(Z₁+Z₂)² Eq. (3)

where Z1 and Z2 are the impedances of material 1 and 2, respectively. Based on Equations 2 and 3, the total sum of reflection and transmittance is equivalent to unity (R+T=1); hence, energy is conserved (lossless case). The reflection of waves may be used in medical imaging to visualize internal tissues and organs. When acoustic mismatch is large, most of the waves are bounced back from the interface (e.g., as a strong echo) and the remaining energy, e.g., the energy transmitted into the second medium, cannot be used to produce images for inner organs and tissues. This is why a transducer cannot acquire an image when there is a gap of air between the transducer and the patient's skin (e.g., total reflection of acoustic waves due to impedance discontinuity). Thus, a material with an intermediate acoustic impedance (e.g., gel or oil) may be placed between the transducer and the skin while imaging. Such acoustic mismatch is the reason that air-filled organs (e.g., the lungs) may block imaging or visualization of underlying tissues and organs. Ultrasound frequencies used for such medical imaging applications usually range between about 0.8 and 10 MHz. Frequencies in this range may be used in one or more of the methods disclosed herein where HFUS is used. One or more of the methods disclosed herein uses HFUS having a frequency of about 1-3 MHz.

### Ultrasound Triggering

Ultrasound may have advantages over other release and/or triggering techniques. It is widely employed in the medical field due to its low cost, safety and simplicity. An exposure to high-intensity, high-frequency US for 2 minutes can result in a temperature elevation of 4 to 5° C., which is generally accepted to be below cytotoxicity levels. Ultrasound has the ability to propagate into deep tissue, while optical waves cannot. In addition, US can be controlled in time and space, and focused directly on the tumor cells while minimizing effects on healthy tissues. Furthermore, US is noninvasive, hence no surgery or insertion is needed. Indeed, US may enhance drug delivery to solid tumors up to 10-fold, when compared to delivery in its absence. Finally, US-actuated targeting may be synergistic with other delivery methods, both passive and active, to advantageously improve/augment the desired effect(s) on tumor tissues. The synergism may occur, at least in part, due to cavitation effects.

Triggering in drug delivery may allow a certain degree of control over one or more factors of drug release, e.g., amount, location, and the period at which a drug is released. Various triggering techniques may be used to trigger release liposome-encapsulated drugs, e.g., pH, temperature, enzymes, and light stimuli. In addition drug release may be efficiently controlled using US.

In liposomal drug delivery, US may be used to control the release of a certain drug via mechanical and/or thermal effects. Ultrasound-triggered release may be advantageously applied to echogenic and emulsion liposomes (eLiposomes). Two parameters of US that may be involved in triggering the release of drugs (e.g., the release of drugs from liposomes) include frequency and intensity, as discussed herein. In drug delivery, high-intensity focused US (HIFU) may be advantageously used to trigger release of drugs from temperature-sensitive liposomes. HIFU can also treat (kill) tissues at elevated temperatures, a process known as hyperthermia. Hyperthermia occurs when a US beam is focused on a tissue volume (e.g., a small tumor tissue volume) and the power per area (called intensity or power density) increases (e.g., becomes very high), resulting in localized heating (e.g., heating of the tissue volume). Depending on the tissues involved, e.g., healthy tissue and/or cancerous tissue, hyperthermia-induced tissue destruction may be a desirable outcome. Release of drugs can also be achieved using the non-thermal effects of low frequency US (LFUS). For example, exposure to LFUS may advantageously increase the permeability of biological barriers (e.g., skin, cell membranes, and tumors) and thereby facilitate extravasation of a drug into the desired site. Additionally, cell membranes can be disrupted (e.g., temporarily disrupted) via LFUS-induced cavitation. The LFUS results in small, transient, pore-like disruptions (e.g., holes) in the membrane (e.g., having diameters smaller than about 28 nm). These transient disruptions generally last for several minutes, after which the cellular repair mechanism recovers the normal, intact membrane configuration. HFUS waves may be easily focused whereas LFUS waves are generally more diffuse and difficult to focus. Therefore, HFUS ability to effectively target infected tissues without affecting the surrounding tissues may make HFUS preferable (over LFUS) in the field of drug delivery. Three mechanisms that may lead to a change in cellular accumulation of a molecule loaded in a nanoparticle include: 1) enhancement of the release before the nanoparticles reaches the cell, 2) enhancement of cellular uptake mainly by endocytosis and 3) sonoporation of the cell membrane. Cavitation (mostly transient cavitation), may be the main driver of release and cellular uptake. However, radiation force-induced acoustic streaming may play a role as well. Additionally, the increase in the enthalpy of drug internalization after US exposure may also contribute to an incrementally increased uptake of small drugs loaded in nanoparticles.

Several parameters may affect the release of drugs from nanoparticles. For example, in vitro, LFUS may have the ability to release contents—an effect that may be enhanced by increasing power density. The mechanical index is another parameter may indicate that release of the nanoparticles contents increases with decreasing frequency and increasing power density. Release may also be affected by changes in the duty cycle as well. Pulsed US exposure may reduce the temperature increase in tissue caused by energy dissipation (versus continuous US wave exposure) limiting the thermal effects of US and limiting healthy tissue damage. Additionally, exposure to LFUS may be able to increase the permeability of biological barriers. This phenomenon may be exploited to increase the permeability of many biological barriers, such as cell membranes and tumors, thus facilitating the extravasation of the drug molecules into the desired site. Finally, cellular membranes can be disrupted via LFUS-induced cavitation. The LFUS tends to result in transient pore-like disruptions in the membrane with diameters lower than about 28 nm that may last for a duration of several minutes, after which the cellular repair mechanism recovers the intact membrane configuration.

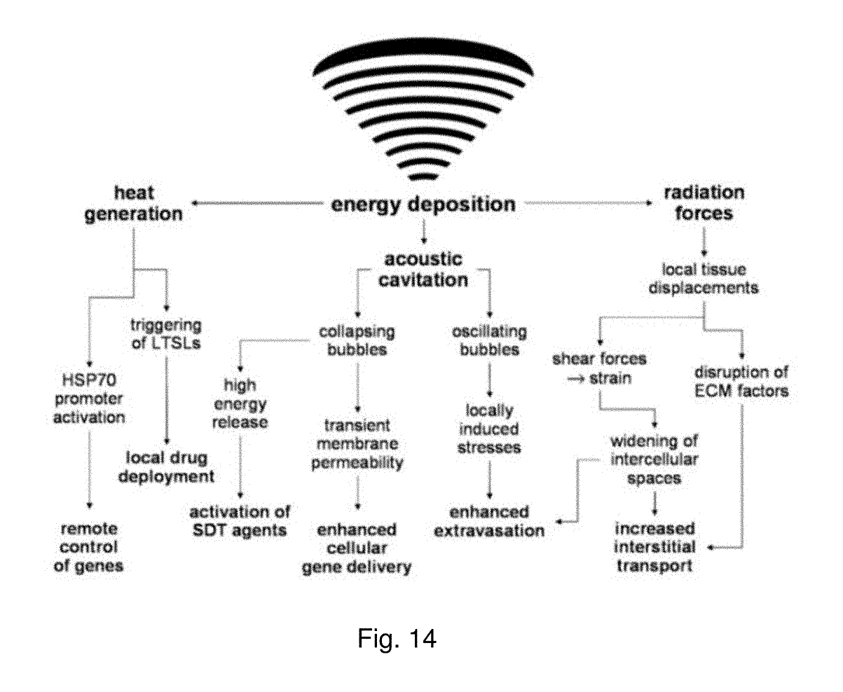

The energy of US, when focused on a specific area, will usually dissipate in heat generation, acoustic cavitation, and radiation forces. FIG. 14 is a schematic that illustrates possible modes of US energy deposition (note—HSP is an acronym for “Heat Shock Protein”, LTSL is an acronym for “Low Temperature Sensitive Liposomes”, SDT is an acronym for “Sonodynamic Therapy”, and ECM is an acronym for “Extracellular Matrix”). In targeted drug delivery, US is generally used to induce at least one of two phenomena: hyperthermia and cavitation. When using liposomes as a DDS, increasing the temperature (e.g., inducing hyperthermia) beyond the phase transition temperature of the lipid may cause the lipid bilayer to open (e.g., delaminate) and release the encapsulated drug. The thermal energy resulting from US-induced heating may be employed in releasing the drug encapsulated in liposomes as well as in ablating the tumor.

Ultrasound may induce mechanical and/or thermal effects. The mechanical effect of ultrasound is reflected in acoustic cavitation events. Acoustic cavitation refers to the formation and/or the activity of gas or bubbles in a medium being exposed to an oscillating pressure. The bubbles are either originally present in the liquid, or may be newly formed when the pressure is lowered below the vapor pressure of the liquid. There are two types of acoustic cavitation: stable and collapse.

Stable cavitation describes the continuous oscillation of bubbles being exposed to an oscillating pressure, which in turn varies the bubble radius about an equilibrium value. The oscillation of bubbles shears the fluid and/or surfaces nearby. The highest oscillation amplitude occurs when the applied US frequency matches the natural, resonant frequency of the bubbles. Stable cavitation has no known negative biological attributes and can be applied to enhance the convection of oxygen and nutrients into normal cells.

Collapse cavitation, also known as transient cavitation, involves bubble oscillations with increasing amplitudes that lead to continuous expansion until exceeding the bubble resonant radius. After the bubble oscillations have exceeded the bubble resonant radius, the bubble grows suddenly and collapses vigorously. The bursting bubbles in collapse/transient cavitation generate a short-lived, intense local heating (e.g., up to about 5000 K) and are accompanied by high pressures (e.g., as high as 1000 atm). Although these very high temperatures can be achieved at a certain point, heating/cooling rates are very fast on the order of 10¹⁰ K/s, thus characterizing the cavitation as an adiabatic event. Shortly after the bubble bursts, a high-velocity (e.g., several hundred meters per second) micro jet of liquid is produced that pushes surfaces nearby with massive energy. The resonant size of a bubble is dependent on the type of gas enclosed by the bubble and the characteristics of the US wave. To initiate collapse/transient cavitation, an ultrasound power density threshold that is both proportional to frequency and a characteristic of the bubble size must be achieved. As frequency decreases, the power density threshold also decreases, which explains why collapse/transient cavitation may occur more frequently at low frequencies rather than at high frequencies. LFUS with high power density may induce collapse/transient cavitation. The consequences of collapse/transient cavitation may be detrimental to tissues and adjacent cells due to the shear stress (e.g., potentially huge shear stress), the shockwave produced, and the free radicals that may be generated at elevated temperatures (note, such free radicals may interfere with biochemical processes. LFUS waves used in the methods disclosed herein may generally be in the range of about 1-30 kHz, about 2-28 kHz, about 3-26 kHz, about 4-24 kHZ, about 5-22 kHz, about 6-20 kHz, about 7-18 kHz, about 8-16 kHz, about 10-14 kHz, or any other frequency that causes advantageous release profiles and/or other beneficial results according to the methods disclosed herein.

In liposomal drug release, stable cavitation may not be as effective as collapse/transient cavitation. Drawbacks of using collapse/transient cavitation (e.g., in drug release and/or DDS) may be advantageously minimized by selecting suitable parameters for the therapeutic US: it may be desirable to produce bubble activity that effectively ruptures the liposomal membrane without damaging the adjacent endothelial cells or causing thrombosis. The mechanical index (MI) may help determine the occurrence of collapse/transient cavitation. MI relates the peak negative pressure (Pᵣ) during the rarefaction cycle of the wave to the square root of the wave frequency (fₒ) according to the following equation:

$$MI = \frac{{P}_{r}}{\sqrt{{f}_{0}}} \qquad Eq . ( 4 )$$

Using US as a trigger, it may be possible to control certain factors that influence the release of the drug, such as, but not limited to, power input, ultrasonic intensity, duration of sonication, and position of the US source.

As discussed herein, each of targeted liposomes, albumin-targeted liposomes, and US may have an effect, e.g., a significant effect, on drug release and uptake. The following are representative examples of such phenomena.

Delivery of the anticancer drug Dox from folate-targeted liposomes may be evaluated by measuring the change in Dox fluorescence using a spectrofluorometer. The uptake of folate-PEG-liposomes by cancer cells (e.g., KB human oral carcinoma cells) may be higher than (e.g., significantly higher, up to, and including about 45 times higher) that of non-targeted liposomes.

PEGylated-liposomes targeted with albumin (PEG-L-A) may be used for macrophage-specific drug delivery. Macrophages may contribute to tumor growth; hence, effective targeting of drugs to such cells may provide an approach for the treatment of some types of cancer. The drug uptake by macrophage cells may be evaluated using fluorescence microscopy and flow cytometry. Macrophage uptake of PEG-L-A may be higher (e.g., about 17-fold higher) after a period of incubation (e.g., 1 hour of incubation) and can remain higher (e.g., about 4 times higher) after 24 hours, when compared to that of plain liposomes. Moreover, macrophage uptake of PEG-L-A may be higher (e.g., significantly higher) than the uptake of PEGylated-liposomes. For example, macrophage uptake of PEG-L-A may be about 53 times higher than that of PEGylated liposomes after about 1 hour and about 9 times higher than that of PEGylated liposomes after about 24 hours. In addition, different types of albumin coated liposomes, such as murine (MSA), bovine (BSA) and human serum albumin (HSA) may be used. However, the uptake of liposomes coated with each type of albumin may be independent of the antigenicity of a foreign protein—no significant differences are observed when comparing the uptake of liposomes targeted with each of the three different types of albumin.

Acoustically active liposomes (AAL), regardless of contents (e.g., containing air bubbles and/or calcein) generally release at higher concentrations than other, non-AAL, types of liposomes, including air-only liposomes and calcein-only liposomes. High frequency US (HFUS) may effectively trigger release from AAL. AAL generally release at higher concentrations (e.g., much higher concentrations) of drug (or model drug, such as calcein) than non-AAL. For example, AAL release at much higher concentrations than non-AAL using 1-MHz HFUS under different intensities and duty cycles, including, but not limited to, 2 W/cm² and 100% duty cycle for 10 s. Under these conditions, AAL released may peak.

Drug release from liposomes triggered by US may be affected by US and/or the chemical nature of liposome constituents. For example, parameters of US affecting drug release include, but are not limited to, wave frequency and power intensity. Factors related to liposome nature include, but are not limited to, lipid ratio, amount of grafted PEG, surface charge, etc.

Drugs, such as Doxorubicin (Dox) from Doxil®, may be released from AAL using different US frequencies. Doxil® is a liposomal anti-cancer drug that contains Dox loaded into stealth liposomes with a diameter of about 100 nm. At the time of this writing, Doxil is one of the currently FDA-approved drug delivery formulations. Exposing Doxil® to about 20-kHz (about 1.2 W/cm²) LFUS generally results in release of about 85% in saline and about 61% in human sourced plasma. At higher frequencies, release may slow in saline and nearly stop in plasma. For example, at a frequency of about 1 MHz (about 2.5 W/cm²), release may occur slower in saline and release may nearly stop (e.g., about 5%) in human plasma. At a frequency of about 3 MHz, no release may be observed in either medium. Lower levels of drug release in plasma may be explained, at least partially, by the presence of plasma proteins that may absorb part of the US energy, thus reducing the potential of cavitation in liposomal membranes. In addition, Doxil® may have a higher (e.g., longer) half-life than Dox encapsulated in non-PEGylated liposomes, and also higher (e.g., longer) than that of free Dox.

Release of the model drug calcein (C₃₀H₂₆N₂O₁₃) from DEPC-based liposomes may be higher at low frequency (e.g., about 300 kHz) than at higher frequency (e.g., about 1 MHz). The US frequency may also exhibit some dependence on the size and lamellarities of liposomes. When liposomes are very small compared to the wavelength of US, the pressure gradient is not sensed by the vesicle; instead, a uniform pressure is detected. For this reason, applying high frequency US (e.g., about 1 MHz, λ=1.5 mm)—4 orders of magnitude greater than a 100 nm liposome—may have a similar effect on drug release as applying a LFUS (e.g., about 20 kHz, λ=75 mm)—5 orders of magnitude greater than a 100 nm liposome. However, better release may be achieved in some instances when using LFUS. This may be explained, at least partially, by the fact that LFUS requires a lower power density to initiate collapse/transient cavitation.

US-mediated drug release from sonosensitive liposomes may also depend on the membrane structure of these vesicles. For example, DOPE-based liposomes with DSPE-PEG₂₀₀₀ and cholesterol may achieve higher Dox release upon exposure to LFUS (e.g., about 40 kHz) than DSPE and PC-based liposomes in 20% serum.

The mechanism of drug release from liposomes induced by US may be evaluated using cryogenic transmission electron microscopy (Cryo-TEM). For example, if vesicle morphology and size distribution remain the same, before and after US exposure, sonoporation (pore-mediated release) likely played a role (e.g., a major role) in the release process. If vesicles disintegrate into a smaller size, temperature effects likely played a role (e.g., a predominant role) in the release process. Additionally, inclusion of PEG within the liposomal membrane structure may be found to increase drug release since PEG molecules absorb more US energy than lipid molecules, thereby increasing the events of cavitation.

Release of calcein from estrone-anchored stealth liposomes under the effect of US may be evaluated as a model of a DDS for the treatment of breast cancer, among other conditions. Non-triggered release of drug from this type of liposome, achieved and evaluated over a period of about 24 h (e.g., determined by the dialysis tube method), may be about 53.6±1.23% for the ES-SL-Dox (estrogen targeted, stealth liposomes carrying Dox) as compared to about 47.3±1.34% release from SL-Dox (stealth liposomes carrying Dox).

Coating liposomes with different ligands, such as proteins, may have advantageous effects. Some such proteins include, but are not limited to, immunoglobulin G and fibronectin, and/or opsonins that enhance the hepatic disposition of liposomes. Proteins with dysopsonic activity in drug delivery may also be advantageously used. For example, rat serum albumin (RSA) may be coupled onto the surface of PEGylated liposomes (PEG-L-RSA). Such liposomes may be used to evaluate and compare the hepatic disposition of non-PEGylated liposomes, PEGylated liposomes and PEG-L-RSA in the rat model. The plasma concentration of PEG-L-RSA may be 3 times greater than that of non-PEGylated liposomes. Thus, RSA may advantageously decrease hepatic clearance of liposomes. However, both PEGylated liposomes and PEG-L-RSA may show similar or the same splenic clearance levels. In addition, Dox accumulation in the heart (e.g., cardiac tissue) may be less for PEG-L-HSA than for free Dox.

Drug delivery to mouse tumors may be successfully triggered by US. For example, mice receiving liposome-encapsulated Dox (Doxil®) injection into their tails veins and then exposed to high intensity US. Controls may be used to evaluate the liposome's performance. For example, control mice may be injected with Doxil®, but not exposed to US. Other control mice may receive no treatment. Removal of the tumors and analysis of the Dox accumulation reveals that Dox concentration in tumors of mice receiving both liposome-encapsulated Dox and US can be about 124% higher than the Dox concentration in tumors of mice that received only liposome-encapsulated Dox, without US. Hence, it may be concluded that the delivery of liposomal Dox to tumors treated with high intensity US advantageously improves treatment efficacy.

Mice implanted with prostate tumors may also be used to evaluate liposome (or other treatment) performance. Phthalocyanine chloride tetrasulphonic acid (AlPcS₄) encapsulated in PEGylated liposomes at a tumor site may be exposed to 1.1-MHz US. Release of the compound may be monitored, e.g., by an increase in concentration and/or fluorescence. Fluorescence may be observed to increase up to about 100%, which implies an efficient to perfect release of the drug. Another treatment that may be evaluated is HIFU-induced hyperthermia, including, for example, the effect of HIFU on the drug release from low temperature sensitive liposomes (LTSL). LTSL and free Dox may be administered intravenously in rabbits implanted with VX2 carcinoma cells after which the tumors may be exposed to LTSL. The concentration of Dox is generally higher in tumors exposed to LTSL (e.g., about 8.8±1.4 μg Dox/g tissues) than in tumors treated with free Dox (e.g., about 4.0±1.0 μg Dox/g tissues). The uptake of Doxil® by melanoma BLM tumor implanted in mice may also be evaluated. In this case, the uptake of Doxil® tumor tissue is, again, generally higher than that of free Dox. However, higher uptake does not, necessarily, guarantee the drug's cytotoxicity of the drug: it generally needs to be released to kill cells (e.g., the malignant, cancer cells). Thus, cytotoxicity and bioavailability for both Doxil® and free Dox is useful. Cells (including both human and mice melanoma cells) treated with free Dox for about 8 hours accumulate about 26% of the initial amount in the nucleus (only about 0.5% of the initial concentration was detected in the cell cytoplasm): this concentration/accumulation profile killed the cells effectively. Cells treated with Doxil® (liposome encapsulated Dox) for about 8 hours achieved only about 0.4% accumulation of the available drug in nucleus, thereby revealing a reason for low cytotoxicity of Doxil® when administered without an external stimulus (generally considered necessary to release the entrapped drug).

Female BALB/c mice show a greater uptake for estrone-targeted stealth liposomes in breast and uterus tissues versus the uptake of stealth liposomes without a targeting moiety. The greater accumulation of the liposomal formulation may be attributed to the receptor-mediated endocytosis in response to estrone presence. Antitumor activity on a MCF-7 xenografted in nude mice also suggests that ES-SL-Dox formulation may have the greatest effect in inhibiting the tumor growth.

Emulsion Liposomes (eLiposomes)

Ultrasound, as a triggering technique, generally works best when gas bubbles are present. Stabilized microbubbles may release drug(s) upon exposure to US. However, stable bubbles generally cannot be formed at sizes below about 1 μm. And, larger bubbles generally will not leak through tumors endothelium (via the EPR effect discussed herein); instead, they will usually remain in the circulatory system till they are opsonized. Ultrasound, as an active targeting technique, advantageously provides control over the timing and the rate of drug release. When liposomes are exposed to US, microbubbles close to the liposomes are responsible for the cavitation that shears and ruptures the liposomes. However, since gas bubbles generally do not extravasate into tumor tissues, liposomes may advantageously be highly sensitivity to US without depending on the existence of adjacent gas bubbles. Such highly sensitive liposomes include, but are not necessarily limited to liposomes synthesized using vaporizable emulsion droplets, generally known as emulsion liposomes or eLiposomes. The emulsion in these liposomes is of nanosize and made of perfluorocarbons (e.g., perfluorohexane PFC₆, perfluoropentane PFC₅, etc.) with high vapor pressures at biological temperatures, making liposomes responsive to low US intensities. The normal boiling points for PFC₅ and PFC₆ are 29° C. and 56° C., respectively. Unlike gas bubbles, emulsions using these perfluorocarbons can be stably formed at nanoscale sizes. For example, calcein-encapsulating eLiposomes with a PFC₅ emulsion release barely any calcein at body temperature (e.g., about 37° C.), even though that is above the boiling point of the mentioned emulsion. This is because the apparent boiling point of the emulsion is higher than the boiling point of the individual perfluorocarbon due to the additional pressure, known as Laplace pressure, imposed by the lipid sphere containing the emulsion. When eLiposomes (e.g., calcein-encapsulating eLiposomes with a PFC₅ emulsion) are incubated at higher temperatures (e.g., higher than the boiling point of PFC₅), release may be found to increase significantly. The formation of gas bubbles is the likely mechanism of release for such eLiposomes.

eLiposomes may be combined with US to initiate drug release. When eLiposomes are exposed to US (e.g., during the low pressure phase of the US wave) the local pressure drops below the high pressure of emulsions. That pressure drop is translated to a change in the phase of the emulsion to a vapor phase, which expands and bursts the membrane of the liposome to release some, or all, of the encapsulated drug.

### Nanocarrier Formation and Production

Liposomes may be prepared according to a modified lipid film hydration method as described herein. DSPE-PEG-estrone lipid derivative may be prepared according to the various methods disclosed herein (presence of the targeting moiety may advantageously be detected/confirmed using Infrared Spectroscopy (IR)). The size of liposomes may be determined by Dynamic Light Scattering (DLS). Finally, the US-triggered release of a calcein as a model drug may be evaluated at various frequencies, including, but not limited to low frequency (e.g., about 20 kHz) and high frequency (e.g., about 1.07 MHz and about 3.24 MHz).

In some embodiments, liposomes may be prepared from 1,2-distearoyl-sn-glycero-3-phosphoethanolamine-N-[amino(polyethylene glycol)-2000] (DSPE-PEG₂₀₀₀-NH₂) modified with estrone, and cholesterol, in addition to 1,2-dipalmitoyl-sn-glycerol-3-phosphochloine (DPPC). As explained herein, Poly(ethylene) glycol may play a role (e.g., a critical role) in prolonging the circulation time for nanocarrier encapsulated drugs. The effect of incorporating cholesterol in lipids depends on the type of lipids. Unsaturated lipids' (i.e., with double bonds) high fluidity is suppressed by adding cholesterol; hence, their water permeability decreases and their stability increases. Saturated lipids (e.g., DSPE, DPPC), tend to behave differently when cholesterol is added due to their gel-like consistency, which makes them leak-resistant (low water permeability) even in the absence of cholesterol. The addition of cholesterol to saturated lipids may reduce the lipids' phase transition temperature.

Various types of liposomes may be synthesized and used according to the methods and with the systems disclosed herein. For example, targeted liposomes (with targeting moiety anchored to their surface). In another example, control liposomes (without the targeting moiety) may be used to evaluate the performance of other liposomes (e.g., the targeted liposomes). Synthesis of targeted liposomes according to one or more methods disclosed herein may begin with the reaction of cyanuric chloride (NCCl)₃ (e.g., (2,4,6 trichloro-1,3,5 triazine)) with estrone (ES) to form the targeting moiety ES-N₃C₃Cl₂. Cyanuric chloride may serve as a rapid, simple, and safe way to couple DSPE-PEG to different targeting ligands due to its bifunctionality as a coupling reagent. The first two chloride substitutions of CC can be achieved upon reacting with nucleophiles under slightly basic conditions, the first reaction at 0° C. and the second at room temperature. At the same conditions, the third chloride substitution reaction is strongly depressed and is predicted to occur above 60° C. Then, the previously formed conjugate may be reacted with DSPE-PEG₂₀₀₀-NH₂ to form DSPE-PEG₂₀₀₀-N₃C₃Cl-ES, which may then be reacted with DPPC and cholesterol to form liposomes. Synthesis of control liposomes according to one or more methods disclosed herein may be carried out using a similar synthesis process as that used to form targeted liposomes, but the control liposomes may use DSPE-PEG₂₀₀₀-NH₂ instead of DSPE-PEG₂₀₀₀-N₃C₃Cl-ES.

Preparation of DSPE-PEG-NH₂ and DSPE-PEG-N₃C₃Cl₂-ES Liposomes—Synthesis and IR Analysis of Estrone-N₃C₃Cl₂ Chloride Conjugate

ES may be reacted with (NCCl)₃ to form a targeting moiety that may be conjugated to liposome phospholipids (e.g., DSPE-PEG₂₀₀₀-NH₂). The hydroxyl group of ES tends to be a good nucleophile at basic conditions and can substitute (e.g., easily substitute) the first chloride of cyanuric chloride at about 0° C. More importantly, this modification is capable of retaining the binding properties of the estrone conjugate since it is described that the affinity of estrogens to the corresponding receptors is determined by the chemical structure of the ligand between the hydroxyl group at the C3 position and the ketone group at the C17 position.

To assess the reactivity of the hydroxyl group of estrone, IR spectra of estrone, cyanuric chloride and an estrone-cyanuric chloride conjugate may be obtained via the potassium bromide (KBr) disk method and compared. It can be observed that the alcohol group of estrone (with a stretching frequency of 3343.43 cm-1) is not detected in the functionalized estrone, which confirms the formation of the estrone-cyanuric chloride derivative. The presence of estrone-cyanuric chloride may be confirmed by 13C-NMR. In this conjugate, the chemical shift of a carbon from the cyanuric ring increases from 169.9 to 187.0 due to the substitution of a chloride.

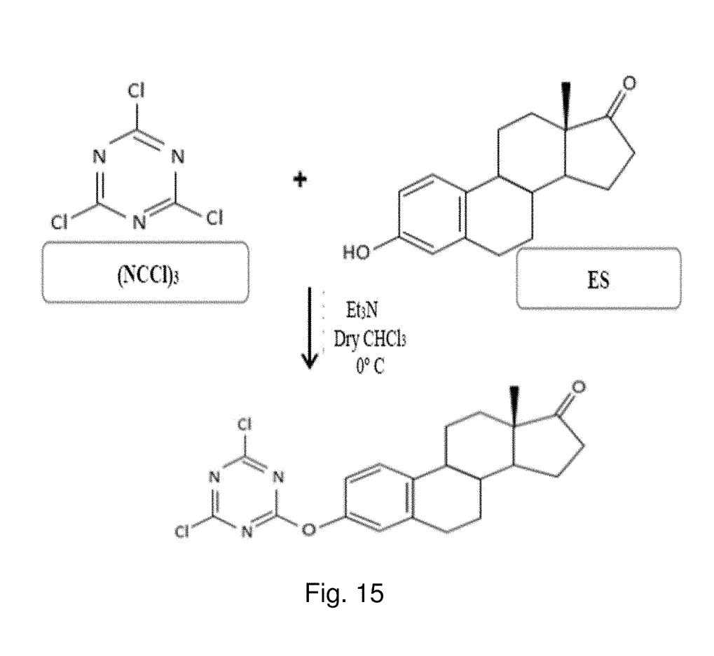

Estrone may be reacted with (NCCl)₃ in a 1:1 molar ratio (of course other molar ratios may be used, ranging from about 0.01:5 to about 5:0.01), in the presence of two molar equivalents of triethylamine (TEA), a chloride acceptor. To achieve the mono-substitution of chlorine, the reaction may be performed at about 0° C. for about three hours. Other temperature and time combinations that allow the mono-substitution of chlorine may be selected. Next, about 2.45 mg of (NCCl)₃ may be dissolved in a small amount of dry chloroform, e.g., in a round bottom flask. Then, a solution of about 3.6 mg of ES dissolved in a suitable amount of dry chloroform may be prepared in a vial, which may then be placed in an ice bath, prior to the addition of about 3.7 μl of TEA. The round bottom flask containing the (NCCl)₃ solution may then be placed in an ice bath with magnetic stirring (e.g., at about 0° C.), and the ES solution added dropwise. The flask may be covered and the mixture continuously stirred (e.g., for about three hours) and cooled (e.g., in an ice bath). Then, the mixture may be left overnight, stirring at about room temperature (stated alternatively, the functionalized ligand 2,4 dichloro, 6 estrone-1,3,5 triazine (CC-ES) may be reacted with the lipids DSPE-PEG₂₀₀₀-NH₂ in a 1:1 molar ratio, in the presence of two equivalents of TEA using chloroform as a solvent, and the reaction may be carried at 0° C. for three hours, then the mixture may be left stirring overnight at about room temperature.). Finally, the chloroform may dried under noble gas (e.g., argon) and the ES-N₃C₃Cl₂ conjugate stored until use (e.g., at about −20° C.). The reaction just described is shown in FIG. 15.

The ES-N₃C₃Cl₂ conjugate (shown as the final product in FIG. 15) may be analyzed by IR to confirm that the desired reaction product is obtained. The potassium bromide (KBr) disk method may be used to carry out the analysis. Testing by IR may advantageously be used because the absorbance/transmittance of the hydroxyl group in the estrone molecule can be detected with its corresponding wave number as a peak in the IR spectra. Therefore, if the reaction was successful, the peak of the hydroxyl group will disappear. Before starting the test (e.g., immediately before starting the test), the KBr powder may be dried due to its hygroscopicity, which may adversely affects the analysis. About 150-200 mg of spectrophotometric-grade KBr powder may be ground to fine powder (e.g., in an agate mortar using a pestle). Then, a small amount of ES-N₃C₃Cl₂ conjugate (about 0.1-2% of the total mass of the KBr powder being used) may be added and mixed with the KBr powder (e.g., in the agate mortar). The mixed powder may then be formed into a disc. In some embodiments, the mixed powder is carefully added into the collar of a stainless-steel die assembly and pressed for about 2 minutes (e.g., by placing the die into a hydraulic press (International Crystal Laboratories, Garfield, USA)). The pressure may be relieved gradually (to avoid breaking the formed disk), and the die disassembled to remove the transparent disk. The disc may then be carefully placed in a disk holder to be tested using an IR instrument.

### Preparation of DSPE-PEG-pNP Control Liposomes

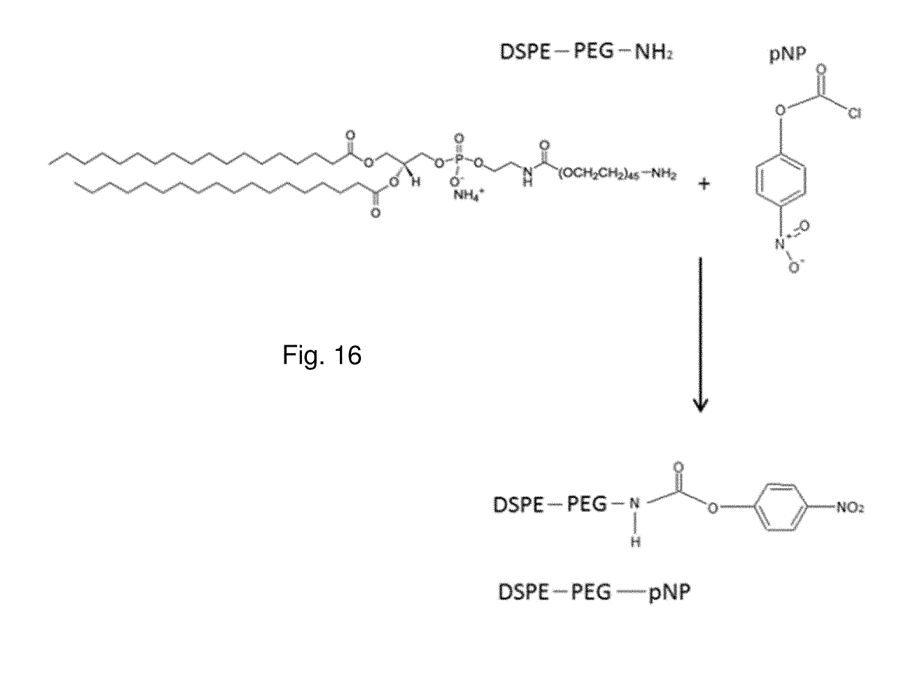

Control liposomes may be prepared using DSPE-PEG-pNP, cholesterol, and DPPC. DSPE-PEG-pNP may be prepared by reacting pNP with DSPE-PEG-NH2, as shown in FIG. 16. The reaction may then be incubated (e.g., overnight at room temperature) with stirring.

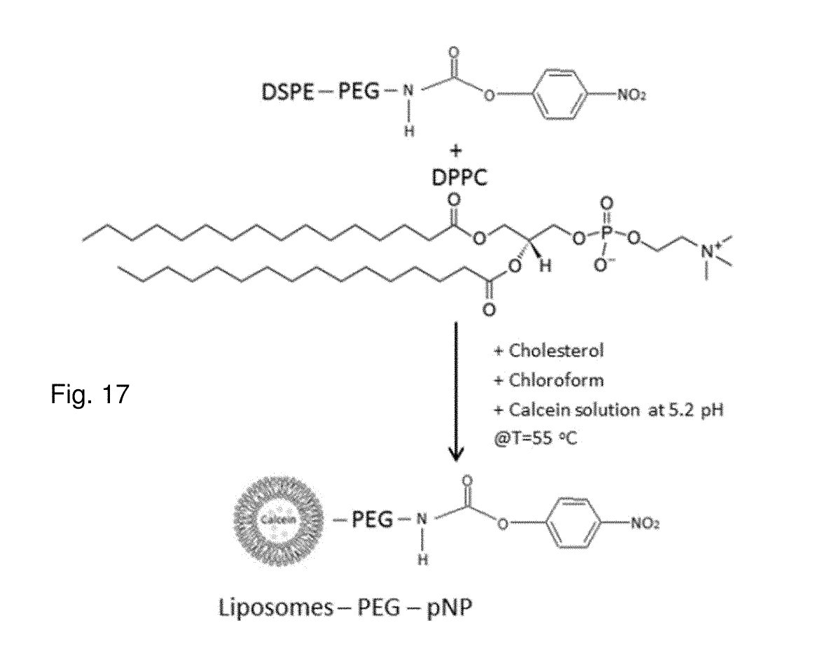

Liposomes may be prepared according to the methods and with the systems disclosed herein (e.g., a modified version of the Torchilin protocol) using DSPE-PEG-pNP, cholesterol and DPPC in a molar ratio of about 5:30:64. The reagents may be dissolved in about 4 ml of chloroform, which may then be evaporated (e.g., under vacuum in a rotary evaporator (Heidolph, Laborota 4003)), until a thin film forms (e.g., on the walls of the flask). The film may be hydrated with about 2 mL of about a 30 mM calcein solution having an adjusted pH of about 5.2. The solution may then be sonicated at about 40 kHz in a sonicator bath (Elma D-78224, Melrose Park, Ill., USA) for about 15 minutes, and then extruded (e.g., about three times (or two, or four, or five) (10× each, changing the filters in between)) (e.g., using the Avanti® Mini-extruder with about 0.2 μm polycarbonate filters (Avanti Polar Lipids, Inc., Alabaster, Ala., USA)). All steps from evaporation to extrusion may be performed at about 55° C., which is above the DSPE transition temperature Tₘ. The transition temperature can be defined as the temperature where structural changes in the lipid membrane occur as it transfers from a gel into its liquid-crystalline phase. The solution may then be cleaned from calcein and salts, e.g., by size-exclusion chromatography (e.g., in a Sephadex G-100 column (GE Healthcare Life Sciences, Pittsburgh, Pa., USA) previously equilibrated with PBS at a pH of about 7.4). Then the turbid liposome fractions may be collected. FIG. 17 shows a schematic of an embodiment of control liposome preparation according to one or more methods described herein. The liposome solution may be kept at about 4° C. in PBS buffer until use.

Preparation of DSPE-PEG2000-NH2 (control) Liposomes Encapsulating Calcein

DSPE-PEG₂₀₀₀-NH₂ control liposomes may be prepared according to methods similar to those described elsewhere herein, such as the method of preparing DSPE-PEG-NH₂ and DSPE-PEG-N₃C₃Cl₂-ES Liposomes. When preparing DSPE-PEG₂₀₀₀-NH₂ control liposomes, two molar equivalents of DSPE-PEG₂₀₀₀-NH₂ may be used instead of DSPE-PEG₂₀₀₀-N₃C₃Cl-ES. A molar ratio of about 65:30:5 DPPC:cholesterol:DSPE-PEG₂₀₀₀-NH₂ may be used.

### Synthesis of DSPE-PEG2000-N3C3Cl-ES

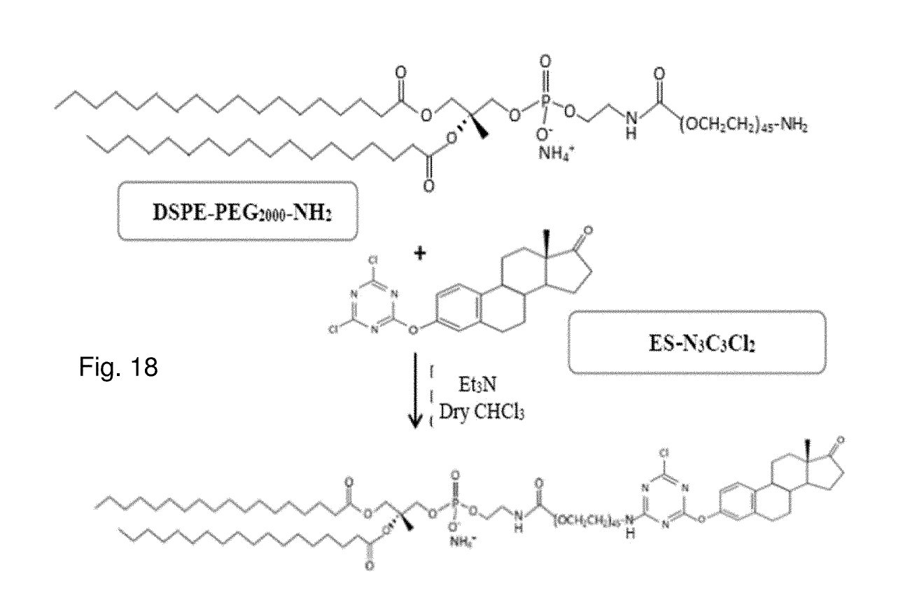

An ES-N₃C₃Cl₂ derivative, such as is described elsewhere herein, may be reacted with DSPE-PEG₂₀₀₀-NH₂ in a molar ratio of about 1.2:1, in the presence of two molar equivalents of TEA. FIG. 18 shows a schematic of this reaction. DSPE-PEG₂₀₀₀-N₃C₃Cl-ES liposomes may be prepared by dissolving about 16.74 mg of DSPE-PEG₂₀₀₀-NH₂ in an amount (e.g., a small amount) of dry chloroform (e.g., in a vial), then adding about 1.66 μl (e.g., about 12 μmol) of TEA (e.g., dropwise) and placing the mixture (e.g., the mixture in the vial) in an ice bath. The ES-N₃C₃Cl₂ conjugate may be dissolved in about 3 ml of dry chloroform and about 1.72 ml (e.g., about 7.2 μmol) transferred to another container (e.g., a small round bottom flask) and chilled (e.g., placed in an ice bath). The DSPE-PEG₂₀₀₀-NH₂ solution may then be added (e.g., dropwise) and the mixture stirred on ice for a time (e.g., for about three hours), after which the stirring continued (e.g., overnight at room temperature). If the prepared lipids are not to be used immediately, the chloroform may be evaporated by purging with a noble gas (e.g., argon) and the result stored below freezing (e.g., at about −20° C.).

### Preparation of DSPE-PEG2000-N3C3Cl-ES Liposomes Encapsulating Calcein

Liposomes may be synthesized at about 60° C. (i.e., above the Tₘ of DSPE) using DPPC, cholesterol, and DSPE-PEG₂₀₀₀-N₃C₃Cl-ES at a molar ratio of about 65:30:5, respectively. Initially, about 2 μmol of DSPE-PEG₂₀₀₀-N₃C₃Cl-ES may be dissolved in about 3 ml of dry chloroform in a container (e.g., in a round bottom flask). Then about 4.7 mg (e.g., about 12 μmol) of cholesterol and about 19.2 mg (e.g., 26 μmol) of DPPC may be added. Chloroform may then be evaporated to dryness (e.g., on a rotary evaporator at about 60° C. for about 15 minutes) until a greasy-like film is formed on the flask walls. The lipidic film may be hydrated with about 2 ml of about 30 mM calcein in phosphate buffered saline (PBS) with the pH adjusted to about 7.4. The solution may then be evaporated (e.g., by rotating in a rotary evaporator without vacuum for about 45 minutes, at about 60° C.). Sonication may then be performed (e.g., in a 40-kHz sonicator bath (Elma D-78224, Melrose Park, Ill., USA) at about 60° C., for about 15 minutes). Then, the sample may be extruded about 30 times at about the same temperature (e.g., using the Avanti® Mini-extruder with 0.2 μm polycarbonate filters (Avanti Polar Lipids, Inc., Alabaster, Ala., USA)). Resultant liposomes may be purified (e.g., using a Sephadex G-100 gel filtration column (GE Healthcare Life Sciences) previously equilibrated with PBS, having a pH of about 7.4). The collected turbid liposome fractions may be labeled and stored at a reduced temperature (e.g., at about 4° C.) until use.

### Preparation of DSPE-PEG2000-N3C3Cl-ES Liposomes Encapsulating Ammonium Sulfate

Liposomes may be synthesized at about 60° C. (i.e., above the Tₘ of DSPE) using DPPC, cholesterol, and DSPE-PEG₂₀₀₀-N₃C₃Cl-ES at a molar ratio of about 65:30:5, respectively. Initially, about 2 μmol of DSPE-PEG₂₀₀₀-N₃C₃Cl-ES may be dissolved in about 3 ml of dry chloroform in a container (e.g., in a round bottom flask). Then about 4.7 mg (e.g., about 12 μmol) of cholesterol and about 19.2 mg (e.g., 26 μmol) of DPPC may be added. Chloroform may then be evaporated to dryness (e.g., on a rotary evaporator at about 60° C. for about 15 minutes) until a greasy-like film is formed on the flask walls. A solution of about 0.11 M ammonium sulfate at a pH of about 5.5 may be prepared to hydrate the dry lipidic film. The lipidic film may be hydrated with about 2 ml of 0.11 M ammonium sulfate solution (e.g., in phosphate buffered saline (PBS)). The solution may then be evaporated (e.g., by rotating in a rotary evaporator without vacuum for about 45 minutes, at about 60° C.). Sonication may then be performed (e.g., in a 40-kHz sonicator bath (Elma D-78224, Melrose Park, Ill., USA) at about 60° C., for about 15 minutes). Then, the sample may be extruded about 30 times at about the same temperature (e.g., using the Avanti® Mini-extruder with 0.2 μm polycarbonate filters (Avanti Polar Lipids, Inc., Alabaster, Ala., USA)). Resultant liposomes may be purified (e.g., using a Sephadex G-25 gel filtration column previously equilibrated with HEPES buffer (0.26 M Sucrose, 0.005 M ascorbic acid and 0.016 M HEPES) using a centrifuge at 1000 rpm for 2 minutes). The collected turbid liposome fractions may be labeled and stored at a reduced temperature (e.g., at about 4° C.) until use.

### Preparation of DSPE-PEG2000-N3C3Cl-ES Liposomes Encapsulating Doxorubicin

The ammonium sulfate gradient method for the encapsulation of doxorubicin, with minor variations, may be used to encapsulate the drug doxorubicin in the targeted liposomes. First, 4 mg of doxorubicin may be added to the liposomal solution providing a doxorubicin to lipid ratio of about 1:6 (w/w). The solution may then be incubated at about 60° C. for about 30 minutes with mild stirring. The resulting solution may then be centrifuged through a Sephadex G-25 gel filtration column using the same protocol discussed elsewhere herein.

### Preparation of DSPE-PEG-Albumin Targeted Liposomes

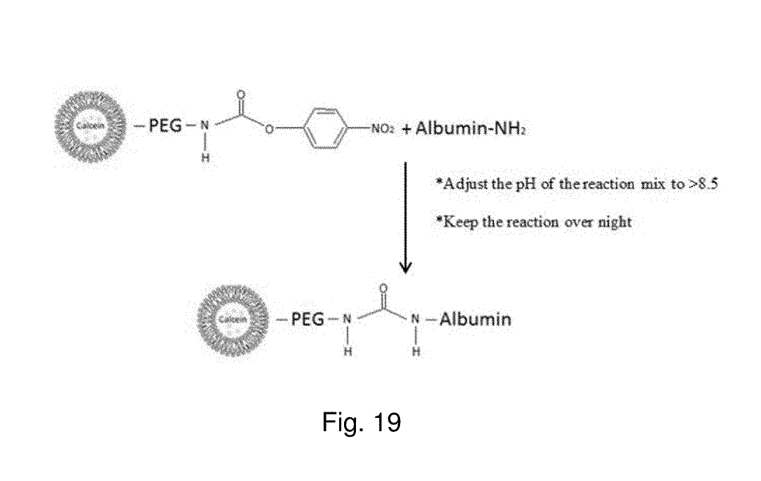

Targeted liposomes may be prepared by reacting DSPE-PEG-pNP control liposomes, as discussed herein, with albumin, as shown in FIG. 19. First, about 0.5 ml of a HSA solution (e.g., about 2 mg/ml in sodium borate buffer at a pH of about 8.5) may be added (e.g., added dropwise) to about 1.5 ml of a DSPE-PEG-pNP liposome solution, as discussed elsewhere herein, in PBS at a pH of about 7.4. The pH may then be adjusted to about 8.5 (e.g., by the addition of a controlled volume of about 1 M NaOH). The reaction mixture may be kept in a container (e.g., a round-bottom flask covered that may be with aluminum foil), and stirred (e.g., stirred overnight at room temperature). The liposome solution may be cleaned (e.g., by size-exclusion chromatography, for example in a Sephadex G-100 column pre-equilibrated with PBS at a pH of about 7.4). The turbid fractions containing the liposomes may be collected and kept until use (e.g., kept at about 4° C. in PBS buffer).

The presence of albumin in a liposome sample may be tested using a BCA assay kit (e.g., a commercially available BCA assay kit). DSPE-PEG-pNP control liposomes may be used as a negative control against which DSPE-PEG-Albumin targeted liposomes may be evaluated.

### Determination of Liposome Size by Dynamic Light Scattering (DLS)

The size distribution of liposomes may be determined in several ways, including, but not necessarily limited to, dynamic light scattering (DLS), electron microscopy, right angle light scattering and turbidity. For example, the mean size of liposomes may be determined and/or evaluated by Dynamic Light Scattering (DLS) (e.g., using the DynaPro® NanoStar™ (Wyatt Technology Corp., Santa Barbara, Calif., USA)). The hydrodynamic radius (Rh) of the liposomes may be determined at about room temperature, after dilution of the liposomes in PBS. DLS measures the intensity of light scattered by the Brownian motion of molecules, as they diffuse, with respect to time. In addition to the hydrodynamic radius, DLS measurements may provide additional information related to size distribution and polydispersity of molecules in solution.

Data obtained from Dynamic7—Static, Dynamic, and Phase Analysis Light Scattering software may be analyzed using two fits: cumulant and regularization. Each fit is generally understood to have different implications and interpretations. A cumulant fit analyzes data under the assumption that particles exhibit unimodal distribution (i.e., they have similar average size). A regularization fit represents data with multimodal particle distribution. A regularization fit may advantageously be used to determine the particle size determination of liposomes due to the multimodality of liposome samples.

### Low Frequency Ultrasound Release

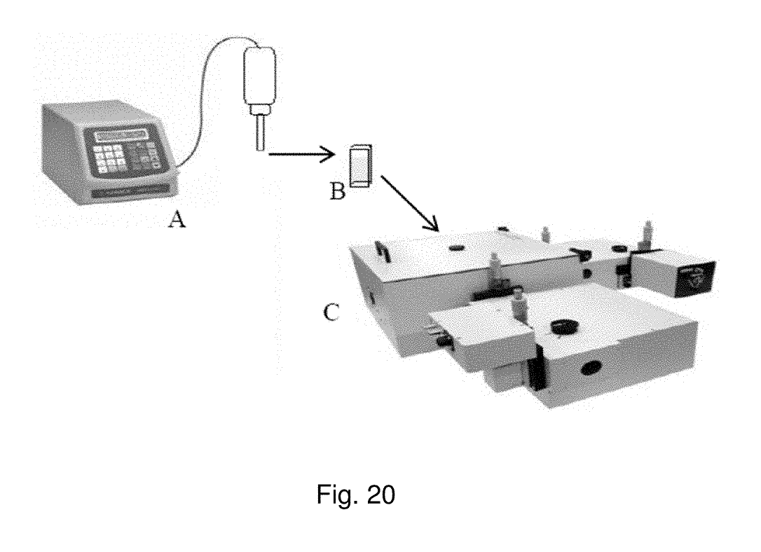

Evaluation of nanocarrier release may be performed online by continuously measuring fluorescence (e.g., using the QuantaMaster QM30 Phosphorescence/Fluorescence Spectrofluorometer (Photon Technology International, Edison N.J., USA)). A drug or a model drug may be used to evaluate nanocarrier release profiles. For example, for various reasons (e.g., the high cost of chemotherapeutic drugs, such as Dox, and for safety reasons), a model drug, such as calcein, may advantageously be used to prepare liposomes and measured to evaluate US-induced release. A spectrofluorometer may be used to measure changes in calcein fluorescence over time, at an excitation wavelength of about 494 nm and emission at about 515 nm (such conditions may provide optimal for liposome-encapsulated calcein). Similarly, release of doxorubicin from liposomes may be monitored at excitation and emission wavelengths of 488 and 581 nm, respectively. A sample dilution may be made directly in a fluorescence cuvette, which can then be placed in a sample chamber inside the spectrofluorometer. An ultrasonic probe (e.g., a 20-kHz ultrasonic probe such as model VC130PB, Sonics & Materials Inc., Newtown, Conn., USA) may be introduced through the opening in the top-cover of the fluorometer and immersed inside the sample, (e.g., about 2 mm into the liquid in the cuvette). FIG. 20 illustrates an apparatus that may be used to evaluate release of a drug, such as a fluorescent drug, including an ultrasonic probe (e.g., a 20-kHz ultrasonic probe) (A), a fluorescence cuvette (B), and a fluorescence spectrometer (C). The initial fluorescence baseline (F₀)—before sonication and release of the drug started—may be recorded for about 60 seconds (e.g., to serve as a baseline or control). The US treatment may then be started and the fluorescence over time (e.g., the fluorescence increase over time) (F) monitored until it reaches a constant value (e.g., sonication may be turned on in a pulsed mode with a 20 seconds “on” and 10 seconds “off” cycle). After reaching a constant value, or maximum release, the sonication may be stopped and detergent (e.g., the detergent Triton X-100 (Tx100)) added to fully lyse the liposomes and release the total amount of encapsulated calcein (F_{Tx}) (e.g., corresponding to 100% release). The percentage of calcein release at each time point may then be calculated using the following equation:

$$\% Release = \frac{F - {F}_{0}}{{F}_{Tx} - {F}_{0}} * 100 \% \qquad Eq . ( 5 )$$

In the above equation, F is the fluorescence intensity at time (t) of insonation, Fₒ is the average of the initial fluorescence intensity before exposing the sample to US, and F_{TX-100} is the maximum fluorescence achieved after lysing the liposomes.

Different concentration solutions of Tx100 and liposomes may be used to examine the detergent-to-lipid ratio needed to reach complete lysis of liposomes. Total release may be obtained by adding Tx100 to a final concentration of about 0.48 mM to an insonated sample. Furthermore, the buffer solution selection may affect liposome lysis (e.g., buffer solution selection is critical to the process). A buffer solution having a pH of about 7.4 may advantageously avoid calcein self-quenching and aggregation that tends to occur at acidic pH. Additionally, that is the pH at which the in vitro cellular assays are commonly performed. Hence, release experiments (e.g., release from nanocarrier DDS, such as liposome systems) may advantageously be performed at a pH of about 7.4, using a final Tx100 concentration of about 0.48 mM to lyse the liposomes. Data produced by release experiments may be normalized using Equation 5 and used to study the release. Initial rates of release may be calculated as the percentage of release after a sonication pulse (e.g., a second sonication pulse) (e.g., about 40 s of total sonication). The final percentage of release may be calculated as the maximum normalized value of the stable part of the relevant release curve(s). Several factors may affect the US-induced release from liposomes, including, for example, US pulse duration, power density, and liposome-related factors, such as, but not limited to, their concentration in the sample, gas encapsulated, and composition. In view of the above, optimum release conditions may be ascertained by varying one or more of these parameters, such as, but not limited to, power density and frequency.

Release of calcein from liposomes may be triggered using 20-kHz LFUS, and monitored by fluorescence changes (e.g., monitored using a QuantaMaster QM 30 Phosphorescence Spectrofluorometer (Photon Technology International, Edison N.J., USA)). Calcein, a model drug, is a fluorescein complex (a fluorescence molecule) with excitation and emission wavelengths of 495 and 515 nm, respectively. As discussed herein calcein may be encapsulated as a 30 mM solution, which is advantageously a self-quenching concentration. Once US is applied to the liposomes, calcein is released from the liposomes to the surrounding medium, it is diluted (at least in test conditions) thus relieving the self-quenching. As such, the calcein concentration in solution may be monitored by an increase in fluorescence. In addition, the fluorescence of calcein is dependent on pH: the fluorescence intensity (FI) of calcein decreases is independent of pH between about 6.5-10, but drops below a pH of about 4.5.

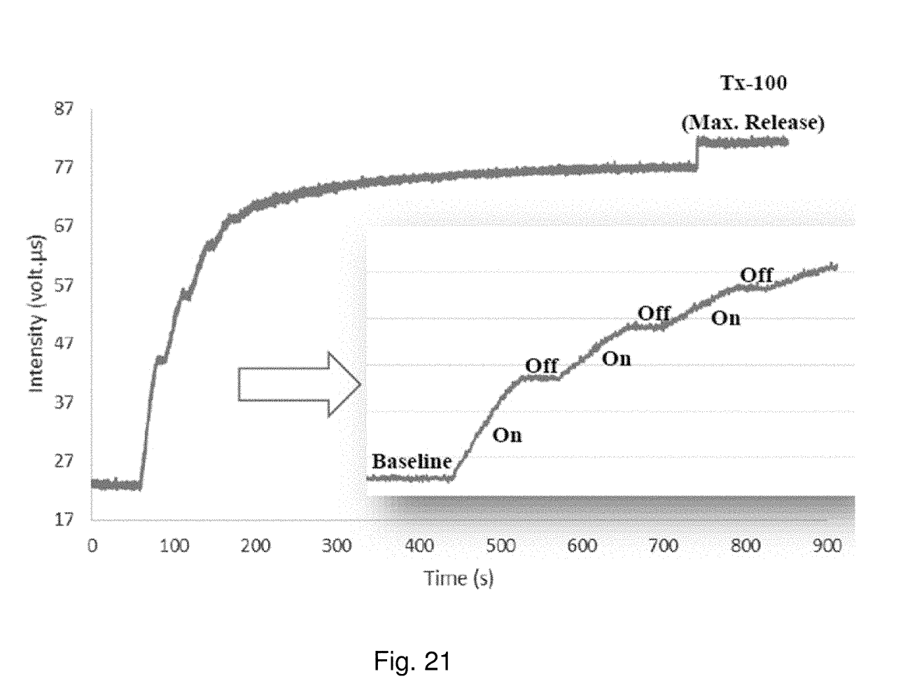

A liposomal suspension may be diluted with PBS buffer (having a pH of about 7.4) in a fluorescence cuvette, which may then be inserted into a spectrofluorometer chamber. An ultrasonic probe (e.g., a 20-kHz ultrasonic probe (model VC130PB, Sonics & Materials Inc., Newtown, Conn.)) may be inserted into the cuvette (e.g., about 2 mm into the cuvette) (e.g., through a specified opening in the spectrofluorometer). Initial fluorescence (F₁) may be recorded for about the first 60 s without sonication to generate a baseline (e.g., a control fluorescence). Then, the liposome suspension may be sonicated (e.g., the sonication may be turned on for a total of about 13 min in a pulsed mode with about 20 s on and about 10 s off). FIG. 21 illustrates an increase in fluorescence level during the “on” cycle (e.g., drug release occurs), while remaining in a plateau during the “off” cycle. Different machine power settings may affect the release from liposomes. Therefore, different machine power settings (e.g., three different machine power settings, including 20%, 25%, 30% which are equivalent to power densities of 6.08, 6.97, and 11.83 W/cm²) may be used to evaluate release. After approaching a maximum liposomal release (a plateau in release that may be reached after about 13 minutes), 2% (w/v) Triton X-100 (Tx100) may be added to the cuvette (e.g., a final concentration of about 0.48 mM) to lyse any remaining, intact liposomes and release all of the encapsulated calcein (e.g., corresponding to fluorescence at 100% release (e.g., a control of 100% release)). The percentage of calcein release may then be determined at any given time using the fluorescence intensity values obtained according to the following equation,

$$\% Drug Release = \frac{F - {F}_{0}}{{F}_{Tx 100} - {F}_{0}} * 100 \% \qquad Eq . ( 6 )$$

where F is the fluorescence intensity at the time (t) of insonation, Fₒ is the average of the initial fluorescence intensity before exposing the sample to US, and F_{Tx100} is the maximum fluorescence achieved after lysing liposomes with Tx100. When comparison analysis is desired, the data may be further normalized.

While 20 kHz is discussed herein as being one example of a low frequency ultrasound that may be used to stimulate nanocarrier disruption and or release, other frequencies may be used. For example, low frequency ultrasound may be used that has a frequency in the range of less than about 500 kHz, less than about 480 kHz, less than about 460 kHz, less than about 440 kHz, less than about 420 kHz, less than about 400 kHz, less than about 380 kHz, less than about 360 kHz, less than about 340 kHz, less than about 320 kHz, less than about 300 kHz, less than about 280 kHz, less than about 260 kHz, less than about 240 kHz, less than about 220 kHz, less than about 200 kHz, less than about 180 kHz, less than about 160 kHz, less than about 140 kHz, less than about 120 kHz, less than about 100 kHz, less than about 90 kHz, less than about 80 kHz, less than about 70 kHz, less than about 60 kHz, less than about 50 kHz, less than about 45 kHz, less than about 40 kHz, less than about 35 kHz, less than about 30 kHz, less than about 25 kHz, less than about 20 kHz, less than about 15 kHz, less than about 10 kHz, or less than about 5 kHz. In some embodiments, low frequency ultrasound may be used that has a frequency in the range of between about 1-50 kHz, between about 2-48 kHz, between about 3-46 kHz, between about 4-44 kHz, between about 5-42 kHz, between about 6-40 kHz, between about 7-38 kHz, between about 8-36 kHz, between about 9-34 kHz, between about 10-32 kHz, between about 11-30 kHz, between about 12-28 kHz, between about 13-26 kHz, between about 14-25 kHz, between about 15-24 kHz, between about 16-23 kHz, between about 17-22 kHz, between about 18-21 kHz, or between about 19-20 kHz. In some embodiments, low frequency ultrasound may be used that has a frequency in the range of between about 50-200 kHz, between about 55-190 kHz, between about 60-180 kHz, between about 65-170 kHz, between about 70-160 kHz, between about 75-150 kHz, between about 80-140 kHz, between about 85-130 kHz, between about 90-120 kHz, or between about 95-110 kHz. In some embodiments, any other low frequency ultrasound may be used that advantageously accomplishes one or more of disrupting the nanoparticles, increasing the localized concentration of drug released, improving the permeability of the target tissue, etc.

While various power densities, including 6.08 W/cm², 6.97 W/cm², and 11.83 W/cm², are discussed herein as being examples of power densities that may be used with high frequency ultrasounds to stimulate nanocarrier disruption and or release, other power densities may be used. For example, a power density may be used that is in the range of less than about 300 W/cm², less than about 295 W/cm², less than about 290 W/cm², less than about 285 W/cm², less than about 280 W/cm², less than about 275 W/cm², less than about 270 W/cm², less than about 265 W/cm², less than about 260 W/cm², less than about 255 W/cm², less than about 250 W/cm², less than about 245 W/cm², less than about 240 W/cm², less than about 235 W/cm², less than about 230 W/cm², less than about 225 W/cm², less than about 220 W/cm², less than about 215 W/cm², less than about 210 W/cm², less than about 205 W/cm², less than about 200 W/cm², less than about 195 W/cm², less than about 190 W/cm², less than about 185 W/cm², less than about 180 W/cm², less than about 175 W/cm², less than about 170 W/cm², less than about 165 W/cm², less than about 160 W/cm², less than about 155 W/cm², less than about 150 W/cm², less than about 145 W/cm², less than about 140 W/cm², less than about 135 W/cm², less than about 130 W/cm², less than about 125 W/cm², less than about 120 W/cm², less than about 115 W/cm², less than about 110 W/cm², less than about 105 W/cm², less than about 100 W/cm², less than about 95 W/cm², less than about 90 W/cm², less than about 85 W/cm², less than about 80 W/cm², less than about 75 W/cm², less than about 70 W/cm², less than about 65 W/cm², less than about 60 W/cm², less than about 55 W/cm², less than about 50 W/cm², less than about 45 W/cm², less than about 40 W/cm², less than about 35 W/cm², less than about 30 W/cm², less than about 25 W/cm², less than about 20 W/cm², less than about 15 W/cm², less than about 10 W/cm², less than about 9 W/cm², less than about 8 W/cm², less than about 7 W/cm², less than about 6 W/cm², less than about 5 W/cm², less than about 4 W/cm², less than about 3 W/cm², less than about 2 W/cm², or less than about 1 W/cm². In some embodiments, a power density may be used that is in the range of between about 0.1-20 W/cm², between about 0.2-19.8 W/cm², between about 0.4-19.6 W/cm², between about 0.6-19.4 W/cm², between about 0.8-19.2 W/cm², between about 1-19 W/cm², between about 1.2-18.8 W/cm², between about 1.4-18.6 W/cm², between about 1.6-18.4 W/cm², between about 1.8-18.2 W/cm², between about 2-17 W/cm², between about 2.2-16.8 W/cm², between about 2.4-16.6 W/cm², between about 2.6-16.4 W/cm², between about 2.8-16.2 W/cm², between about 3-16 W/cm², between about 3.2-15.8 W/cm², between about 3.4-15.6 W/cm², between about 3.6-15.4 W/cm², between about 3.8-15.2 W/cm², between about 4-15 W/cm², between about 4.2-14.8 W/cm², between about 4.4-14.6 W/cm², between about 4.6-14.4 W/cm², between about 4.8-14.2 W/cm², between about 5-14 W/cm², between about 5.2-13.8 W/cm², between about 5.4-13.6 W/cm², between about 5.6-13.4 W/cm², between about 5.8-13.2 W/cm², between about 6-13 W/cm², between about 6.2-12.8 W/cm², between about 6.4-12.6 W/cm², between about 6.6-12.4 W/cm², between about 6.8-12.2 W/cm², between about 7-12 W/cm², between about 7.2-11.8 W/cm², between about 7.4-11.6 W/cm², between about 7.6-11.4 W/cm², between about 7.8-11.2 W/cm², between about 8-10 W/cm², between about 8.2-9.8 W/cm², between about 8.4-9.6 W/cm², between about 8.6-9.4 W/cm², or between about 8.8-9.2 W/cm². In some embodiments, a power density may be used that is in the range of between about 1-100 W/cm², between about 2-95 W/cm², between about 3-90 W/cm², between about 4-85 W/cm², between about 5-80 W/cm², between about 6-75 W/cm², between about-7-70 W/cm², between about 8-65 W/cm², between about 9-60 W/cm², between about 10-55 W/cm², between about 11-50 W/cm², between about 12-45 W/cm², between about 13-40 W/cm², between about 14-35 W/cm², between about 15-30 W/cm², or between about 20-25 W/cm². In some embodiments, any other power density may be used that advantageously accomplishes one or more of disrupting the nanoparticles, increasing the localized concentration of drug released, improving the permeability of the target tissue, etc.

### High Frequency Ultrasound Release Studies

Evaluation of nanocarrier (e.g., liposome) release may also be performed using high frequency ultrasound (e.g., using 1.07-MHz and 3.24-MHz Ultrasonic probes (Precision Acoustics, Dorchester, UK) connected to an AC amplifier (High Voltage Amplifier WMA-300, Falco Systems, Amsterdam, The Netherlands)). Samples may be prepared as discussed elsewhere herein (e.g., a similar procedure to that described in connection with LFUS may be used). Initially, a liposome suspension may be diluted with PBS at a pH of about 7.4, in a fluorescence cuvette. The cuvette may be placed in a fluorometer chamber to read the initial fluorescence intensity (baseline) for about 1 min. Then, the liposome sample may be transferred to another container, such as a beaker (e.g., a small beaker partially immersed in a water bath), at a set/certain distance above the suspended probe (e.g., the liposome sample may be transferred to a small beaker partially immersed in a water bath 4.5 cm above the suspended probe). To avoid losses due to vaporization by US, the container (e.g., beaker) may be covered (e.g., covered with parafilm). The sample may then be sonicated under a continuous mode (in contrast to pulsed mode discussed in connection with LFUS release experiments—of course, other energy deposition protocols may be used), at a frequency and voltage, for about 10 minutes. After sonication, the sample may be transferred to a fluorescence cuvette to record its fluorescence intensity for about 1 min. The process may be repeated. For example, the process may be repeated about every 10 min, for a total insonation time of about 60 min. Afterwards, a detergent, e.g., Tx100, may be added to lyse, e.g., fully lyse, any remaining liposomes and to record the maximum intensity of fluorescence corresponding to the final liposome release.

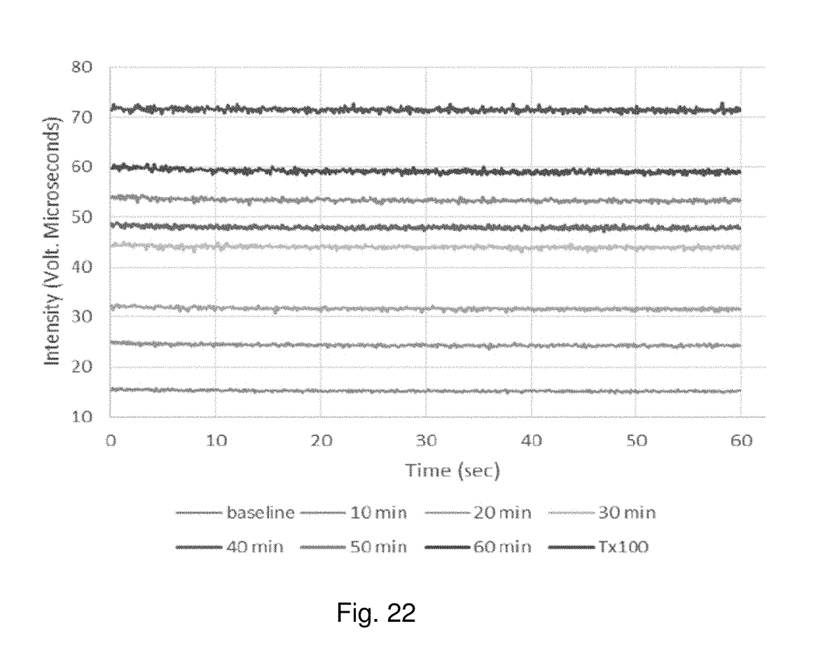

Different machine power settings may affect the release from liposomes. Therefore, different machine power settings (e.g., 1.07 MHz at two or more different machine power settings, including about 10.5 and 50.2 W/cm² and/or 3.24 MHz at one or more power density of approximately 173 W/cm² may be used to evaluate release. The normalized percentage of drug release may be calculated using Equation 5, but the values of Fₜ and F_{Tx100} may be substituted as averages over their period of insonation. FIG. 22 shows various example fluorescence profiles for an experimental replicate followed by one sample calculation.

While various frequencies, including 1.07-MHz and 3.24-MHz, are discussed herein as being examples of high frequency ultrasounds that may be used to stimulate nanocarrier disruption and or release, other frequencies may be used. For example, high frequency ultrasound may be used that has a frequency in the range of between about 1-300 MHz, between about 2-290 MHz, between about 3-280 MHz, between about 4-270 MHz, between about 5-260 MHz, between about 6-250 MHz, between about 7-240 MHz, between about 8-230 MHz, between about 9-220 MHz, between about 10-210 MHz, between about 11-200 MHz, between about 12-190 MHz, between about 13-180 MHz, between about 14-170 MHz, between about 15-160 MHz, between about 16-150 MHz, between about 17-140 MHz, between about 18-130 MHz, between about 19-120 MHz, between about 20-110 MHz, between about 21-100 MHz, between about 22-90 MHz, between about 23-80 MHz, between about 24-70 MHz, between about 25-60 MHz, between about 26-50 MHz, between about 27-40 MHz, or between about 28-30 MHz. In some embodiments, high frequency ultrasound may be used that has a frequency in the range of between about 1-5 MHz, between about 1.1-4.8 MHz, between about 1.2-4.6 MHz, between about 1.3-4.4 MHz, between about 1.4-4.2 MHz, between about 1.5-4 MHz, between about 1.6-3.8 MHz, between about 1.7-3.6 MHz, between about 1.8-3.4 MHz, between about 1.9-3.2 MHz, between about 2-3 MHz, between about 2.1-2.8 MHz, between about 2.2-2.6, or between about 2.3-2.4 MHz. In some embodiments, high frequency ultrasound may be used that has a frequency in the range of between about 1-10 MHz, between about 1.2-9.8 MHz, between about 1.4-9.6 MHz, between about 1.6-9.4 MHz, between about 1.8-9.2 MHz, between about 2-9 MHz, between about 2.2-8.8 MHz, between about 2.4-8.6 MHz, between about 2.6-8.4 MHz, between about 2.8-8.2 MHz, between about 3-8 MHz, between about 3.2-7.8 MHz, between about 3.4-7.6 MHz, between about 3.6-7.4 MHz, between about 3.8-7.2 MHz, between about 4 7 MHz, between about 4.2-6.8 MHz, between about 4.4-6.6 MHz, between about 4.6-6.4 MHz, between about 4.8-6.2 MHz, between about 5-6 MHz, between about 5.2-5.8 MHz, or between about 5.4-5.6 MHz. In some embodiments, high frequency ultrasound may be used that has a frequency in the range of between about 10-80 MHz, between about 11-78 MHz, between about 12-76 MHz, between about 13-74 MHz, between about 14-72 MHz, between about 15-70 MHz, between about 16-68 MHz, between about 17-66 MHz, between about 18-64 MHz, between about 19-62 MHz, between about 20-60 MHz, between about 21-58 MHz, between about 22-56 MHz, between about 23-54 MHz, between about 22-52 MHz, between about 23-50 MHz, between about 24-48 MHz, between about 25-46 MHz, between about 26-44 MHz, between about 27-42 MHz, between about 28-40 MHz, between about 29-38 MHz, between about 30-36 MHz, or between about 32-34 MHz. In some embodiments, high frequency ultrasound may be used that has a frequency in the range of between about 80-150 MHz, between about 82-145 MHz, between about 84-140 MHz, between about 86-135 MHz, between about 88-130 MHz, between about 88-125 MHz, between about 90-120 MHz, between about 92-115 MHz, between about 94-110 MHz, between about 96-105 MHz, or between about 98-100 MHz. In some embodiments, high frequency ultrasound may be used that has a frequency in the range of between about 100-300 MHz, between about 110-290 MHz, between about 120-280 MHz, between about 130-270 MHz, between about 140-260 MHz, between about 150-250 MHz, between about 160-240 MHz, between about 170-230 MHz, between about 180-220 MHz, or between about 190-210 MHz. In some embodiments, any other high frequency ultrasound may be used that advantageously accomplishes one or more of disrupting the nanoparticles, increasing the localized concentration of drug released, improving the permeability of the target tissue, etc.

While various power densities, including 10.5 W/cm², 50.2 W/cm², and 173 W/cm², are discussed herein as being examples of power densities that may be used with high frequency ultrasounds to stimulate nanocarrier disruption and or release, other power densities may be used. For example, a power density may be used that is in the range of less than about 300 W/cm², less than about 295 W/cm², less than about 290 W/cm², less than about 285 W/cm², less than about 280 W/cm², less than about 275 W/cm², less than about 270 W/cm², less than about 265 W/cm², less than about 260 W/cm², less than about 255 W/cm², less than about 250 W/cm², less than about 245 W/cm², less than about 240 W/cm², less than about 235 W/cm², less than about 230 W/cm², less than about 225 W/cm², less than about 220 W/cm², less than about 215 W/cm², less than about 210 W/cm², less than about 205 W/cm², less than about 200 W/cm², less than about 195 W/cm², less than about 190 W/cm², less than about 185 W/cm², less than about 180 W/cm², less than about 175 W/cm², less than about 170 W/cm², less than about 165 W/cm², less than about 160 W/cm², less than about 155 W/cm², less than about 150 W/cm², less than about 145 W/cm², less than about 140 W/cm², less than about 135 W/cm², less than about 130 W/cm², less than about 125 W/cm², less than about 120 W/cm², less than about 115 W/cm², less than about 110 W/cm², less than about 105 W/cm², less than about 100 W/cm², less than about 95 W/cm², less than about 90 W/cm², less than about 85 W/cm², less than about 80 W/cm², less than about 75 W/cm², less than about 70 W/cm², less than about 65 W/cm², less than about 60 W/cm², less than about 55 W/cm², less than about 50 W/cm², less than about 45 W/cm², less than about 40 W/cm², less than about 35 W/cm², less than about 30 W/cm², less than about 25 W/cm², less than about 20 W/cm², less than about 15 W/cm², less than about 10 W/cm², less than about 9 W/cm², less than about 8 W/cm², less than about 7 W/cm², less than about 6 W/cm², less than about 5 W/cm², less than about 4 W/cm², less than about 3 W/cm², less than about 2 W/cm², or less than about 1 W/cm². In some embodiments, a power density may be used that is in the range of between about 1-20 W/cm², between about 2-19 W/cm², between about 3-18 W/cm², between about 4-17 W/cm², between about 5-16 W/cm², between about 6-15 W/cm², between about 7-14 W/cm², between about 8-13 W/cm², between about 9-12 W/cm², or between about 10-11 W/cm². In some embodiments, a power density may be used that is in the range of between about 40-60 W/cm², between about 41-59 W/cm², between about 42-58 W/cm², between about 43-57 W/cm², between about 44-56 W/cm², between about 45-55 W/cm², between about 46-54 W/cm², between about 47-53 W/cm², between about 48-52 W/cm², or between about 49-51 W/cm². In some embodiments, a power density may be used that is in the range of between about 150-225 W/cm², between about 152-220 W/cm², between about 154-215 W/cm², between about 156-210 W/cm², between about 158-205 W/cm², between about 160-200 W/cm², between about 162-195 W/cm², between about 164-190 W/cm², between about 166-185 W/cm², between about 168-180 W/cm², between about 170-175 W/cm², or between about 172-174 W/cm². In some embodiments, a power density may be used that is in the range of between about 1-220 W/cm², between about 5-210 W/cm², between about 10-200 W/cm², between about 15-190 W/cm², between about 20-180 W/cm², between about 25-170 W/cm², between about 30-160 W/cm², between about 35-150 W/cm², between about 40-140 W/cm², between about 45-130 W/cm², between about 50-120 W/cm², between about 55-110 W/cm², between about 60-100 W/cm², between about 65-90 W/cm², or between about 70-80 W/cm². In some embodiments, any other power density may be used that advantageously accomplishes one or more of disrupting the nanoparticles, increasing the localized concentration of drug released, improving the permeability of the target tissue, etc.

### Cellular Uptake of Liposomes

To evaluate the performance of the various liposomes in connection with living tissue(s), it may be useful to test the liposome on cells in vitro. For example, non-targeted calcein liposomes or ES-conjugated calcein liposomes hydrated with 30 mM of calcein may be added to MCF-7 or MDA-MB-231 confluent cells at a concentration of about 200 μM of dipalmitoyl-phosphatidylcholine (“DPPC”). The concentration of DPPC may be estimated using the spectrophotometrical Stewart assay. The liposomes may then be incubated with the cells in 6-well plates for about 30 minutes in humidified air at about 37° C. and about 5% CO₂. Then, the cell plates may be washed with PBS and harvested with a trypsin solution for subsequent Flow Cytometry measurements. The resulting samples may be analyzed to measure calcein fluorescence intensity using an excitation and emission wavelengths of 480 and 520 nm, respectively. Alternatively, the concentration of any other drug contained within the liposome may be evaluated. To evaluate the cellular uptake of liposomes in combination with ultrasound insonation, liposomes (ES-conjugated calcein liposomes) hydrated with about 50 mM of calcein may be incubated with MCF-7 cells under the same conditions discussed in connection with non-conjugated liposomes. After an initial 30 minutes of incubation, the plates may be sonicated while floating in a 40-kHz water bath for about 60 seconds (pulsed sonication at 10 seconds on and 20 seconds off cycles). In some embodiments, no temperature increase is observed in the cell-containing wells during sonication. After US exposure, liposomes and cells may be incubated for about 2.5 hours in humidified air at about 37° C. and about 5% CO₂. Then, the cells may be washed and harvested with a trypsin solution for calcein uptake quantification.

As is discussed herein, drug release from nanoparticles may plateau after a relatively short time. However, some nanoparticles may require more or less sonication to achieve substantially complete disruption and subsequent drug release. In some embodiments, the nanoparticles are sonicated (e.g., ultrasound is applied), with or without pulses as disclosed herein for a time in the range between about 10 seconds to 360 minutes, between about 20 seconds to 350 minutes, between about 33 seconds to 340 minutes, between about 40 seconds to 330 minutes, between about 50 seconds to 320 minutes, between about 1 to 310 minutes between about 1.5 to 300 minutes between about 2 to 290 minutes, between about 2.5 to 280 minutes, between about 3 to 270 minutes, between about 3.5 to 260 minutes, between about 4 to 250 minutes, between about 4.5 to 240 minutes, between about 5 to 230 minutes, between about 5.5 to 220 minutes, between about 6 to 210 minutes between about 6.5 to 200 minutes, between about 7 to 190 minutes, between about 7.5 to 180 minutes, between about 8 to 170 minutes, between about 8.5 to 180 minutes, between about 9 to 170 minutes, between about 9.5 to 160 minutes, between about 10 to 150 minutes, between about 10.5 to 140 minutes, between about 11 to 130 minutes, between about 11.5 to 120 minutes, between about 12 to 110 minutes, between about 12.5 to 100 minutes, between about 13 to 90 minutes, between about 13.5 to 80 minutes, between about 14 to 70 minutes, between about 14.5 to 60 minutes, between about 15 to 50 minutes, between about 15.5 to 40 minutes, or between about 16 to 30 minutes, between about 16.5 to 20 minutes. In other embodiments, the nanoparticles are sonicated (e.g., ultrasound is applied), with or without pulses as disclosed herein for a time less than about 20 minutes, less than about 19.5 minutes, less than about 19 minutes, less than about 18.5 minutes, less than about 18 minutes, less than about 17.5 minutes, less than about 17 minutes, less than about 16.5 minutes, less than about 16 minutes, less than about 15.5 minutes, less than about 15 minutes, less than about 14.5 minutes, less than about 14 minutes, less than 13.5 minutes, less than about 13 minutes, less than about 12.5 minutes, less than about 12 minutes, less than about 11.5 minutes, less than about 11 minutes, less than about 10.5 minutes, less than about 10 minutes, less than about 9.5 minutes, less than about 9 minutes, less than about 8.5 minutes, less than about 8 minutes, less than about 7.5 minutes, less than about 7 minutes, less than about 6.5 minutes, less than about 6 minutes, less than about 5.5 minutes, less than about 5 minutes, less than about 4.5 minutes, less than about 4 minutes, less than about 3.5 minutes, less than about 3 minutes, less than about 2.5 minutes, less than about 2 minutes, less than about 1.5 minutes, less than about 1 minute, less than about 50 seconds, less than about 40 seconds, less than about 30 seconds, less than about 20 seconds, or less than about 10 seconds. In other embodiments, the nanoparticles are sonicated (e.g., ultrasound is applied), with or without pulses as disclosed herein for a time less than about 360 minutes, less than about 350 minutes, less than about 340 minutes, less than about 330 minutes less than about 320 minutes, less than about 310 minutes, less than about 300 minutes, less than about 290 minutes, less than about 280 minutes, less than about 270 minutes, less than about 260 minutes, less than about 250 minutes, less than about 240 minutes, less than about 230 minutes, less than about 220 minutes, less than about 210 minutes, less than about 200 minutes, less than about 190 minutes, less than about 180 minutes, less than about 170 minutes, less than about 160 minutes, less than about 150 minutes, less than about 140 minutes, less than about 130 minutes, less than about 120 minutes, less than about 110 minutes, less than about 100 minutes, less than about 90 minutes, less than about 80 minutes, less than about 70 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, less than about 5 minutes, or any other amount of time that advantageously allows a therapeutically significant amount or quantity of the targeted nanoparticles to collect in a tissue of interest (e.g., a treatment area or cancer tissue).

Control samples may be kept, without sonication, in the same liposome-containing medium at about the same temperature and for about the same time. In each insonation experiment, the sonolysis studies (the effect of US on cell viability) may be performed using a Trypan blue exclusion assay and the cell viabilities evaluated. In some embodiments, cell viabilities may be higher than 90%.

In vivo, such as in the human or another animal body, targeted nanoparticles, such as targeted liposomes (or any other actively or passively targeted nanoparticle) may be allowed to target (e.g., selectively aggregate, accumulate, collect, assemble, amass, pile, etc.) for some time prior to sonication. For example, actively targeted nanoparticles may be allowed to circulate for a sufficient time that a threshold quantity, sufficient to have a therapeutic effect upon the treatment area following sonication-induced disruption, accumulates in or at the treatment area. In some embodiments, the actively targeted nanoparticles are allowed to circulate through the body of the patient for 2 hours prior to application of ultrasound. In some embodiments, the targeted nanoparticles are allowed to circulate through the body of the patient prior to application of ultrasound for a time in the range of between about 1 minute to 48 hours, between about 5 minutes to 46 hours, between about 10 minutes to 44 hours, between about 15 minutes to 42 hours, between about 20 minutes to 40 hours, between about 25 minutes to 38 hours, between about 30 minutes to 36 hours, between about 35 minutes to 34 hours, between about 40 minutes to 32 hours, between about 45 minutes to 30 hours, between about 50 minutes to 28 hours, between about 55 minutes to 26 hours, between about 60 minutes to 24 hours, between about 65 minutes to 22 hours, between about 70 minutes to 20 hours, between about 75 minutes to 18 hours, between about 80 minutes to 16 hours, between about 85 minutes to 14 hours, between about 90 minutes to 12 hours, between about 95 minutes to 10 hours, between about 100 minutes to 8 hours, between about 105 minutes to 6 hours, between 110 minutes to 4 hours, or between 115 minutes to 2 hours. In other embodiments, the targeted nanoparticles are allowed to circulate through the body of the patient prior to application of ultrasound for a time less than about 170 hours, less than about 165 hours, less than about 160 hours, less than about 155 hours, less than about 150 hours, less than about 145 hours, less than about 140 hours, less than about 135 hours, less than about 130 hours, less than about 125 hours, less than about 120 hours, less than about 115 hours, less than about 110 hours, less than about 105 hours, less than about 100 hours, less than about 95 hours, less than about 90 hours, less than about 85 hours, less than about 80 hours, less than about 75 hours, less than about 70 hours, less than about 65 hours, less than about 60 hours, less than about 55 hours, less than about 50 hours, less than about 45 hours, less than about 40 hours, less than about 38 hours, less than about 36 hours, less than about 34 hours, less than about 32 hours, less than about 30 hours, less than about 28 hours, less than about 26 hours, less than about 24 hours, less than about 22 hours, less than about 20 hours, less than about 18 hours, less than about 16 hours, less than about 14 hours, less than about 12 hours, less than about 10 hours, less than about 8 hours, less than about 6 hours, less than about 4 hours, or less than about 2 hours. In other embodiments, the targeted nanoparticles are allowed to circulate through the body of the patient prior to application of ultrasound for a time less than about 720 minutes, less than about 700 minutes, less than about 680 minutes, less than about 660 minutes, less than about 640 minutes, less than about 620 minutes, less than about 600 minutes, less than about 580 minutes, less than about 560 minutes, less than about 540 minutes, less than about 520 minutes less than about 500 minutes, less than about 480 minutes, less than about 460 minutes, less than about 440 minutes, less than about 420 minutes, less than about 400 minutes less than about 380 minutes, less than about 360 minutes, less than about 350 minutes, less than about 340 minutes, less than about 330 minutes less than about 320 minutes, less than about 310 minutes, less than about 300 minutes, less than about 290 minutes, less than about 280 minutes, less than about 270 minutes, less than about 260 minutes, less than about 250 minutes, less than about 240 minutes, less than about 230 minutes, less than about 220 minutes, less than about 210 minutes, less than about 200 minutes, less than about 190 minutes, less than about 180 minutes, less than about 170 minutes, less than about 160 minutes, less than about 150 minutes, less than about 140 minutes, less than about 130 minutes, less than about 120 minutes, less than about 110 minutes, less than about 100 minutes, less than about 90 minutes, less than about 80 minutes, less than about 70 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, less than about 5 minutes, or any other amount of time that advantageously allows a therapeutically significant amount or quantity of the targeted nanoparticles to collect in a tissue of interest (e.g., a treatment area or cancer tissue).

### Liposome Performance and Characterization

Before preparation of liposomes, cyanuric chloride may be attached to DSPE-PEG-NH₂ and estrone. Cyanuric chloride may be used to perform a di-substitution. First, the targeting moiety, ES-N₃C₃Cl₂, may be tested using an IR instrument, as disclosed herein. Then, the particle size of the liposomes, e.g., control and ES-conjugated liposomes, may be determined as discussed herein, e.g., using DLS measurements. The effect(s) of low frequency and high frequency ultrasound on nanocarriers (e.g., liposomes) may be used and/or analyzed to examine any effect that power intensity (e.g., different power intensities) and/or the role of the targeting moiety on drug release. Additionally, the type of cavitation incidents behind the release phenomenon may be determined.

### Infrared Spectra of the ES-N3C3Cl2 Conjugate

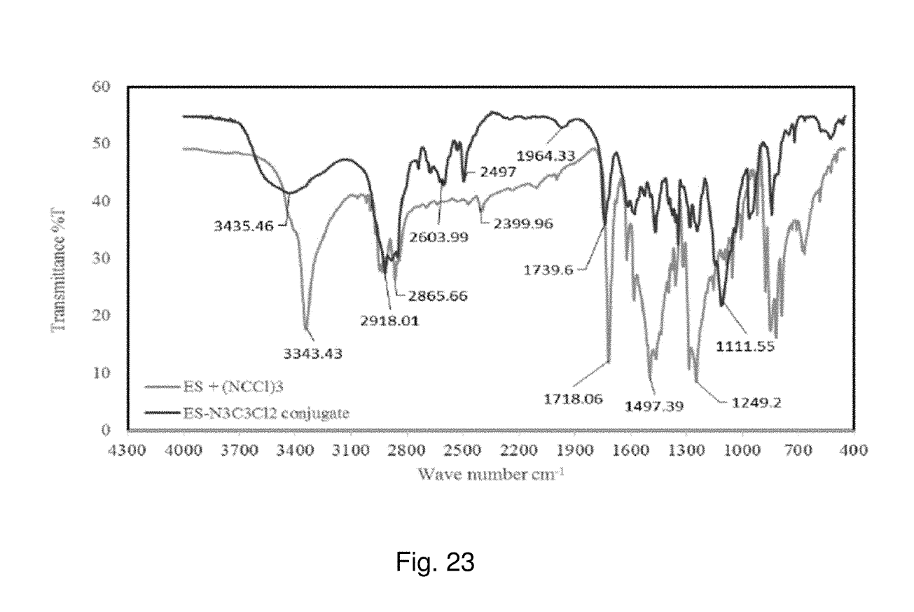

Infrared spectra of estrone, cyanuric chloride and estrone-cyanuric conjugate may provide information regarding reactivity of the hydroxyl group in estrone and to confirm whether the targeting moiety (estrone-cyanuric) was successfully synthesized before proceeding with any of the methods disclosed herein (e.g., reacting with DSPE-PEG₂₀₀₀-NH₂). FIG. 23 illustrates IR spectra of the reaction product (estrone-cyanuric) and reactants (estrone and cyanuric chloride). Absence of the alcohol group in estrone (at a stretching frequency of 3343.43 cm⁻¹) confirm the formation of the estrone-cyanuric conjugate.

The presence of albumin in certain targeted liposomes may be tested using the BCA assay, as discussed herein. DSPE-PEG-pNP liposomes may be used as a negative control to compensate for interference that the assay may have with other liposomal components. After ensuring that the targeting moiety (e.g., protein) is attached, the size of the liposomes (e.g., DSPE-PEG-pNP control liposomes and/or albumin targeted liposomes) may be determined by DLS.

### Particle Size Measurements by DLS

Liposome size (e.g., the size of control and/or ES-conjugated liposomes) may be determined by DLS as described herein. DLS results may be provided by averages±standard deviation liposomes (e.g., of several different batches, e.g., 3 different batches, of liposomes). Based on the regularization fit, ES-conjugated liposomes, as discussed herein, may have a radius of about 92.9±15.8 nm. And, control liposomes, as discussed herein, may have a radius of about 96.75±3.83 nm. There is no statistically significant difference between the sizes of both of these types of liposomes (p>0.7). The average polydispersity percentage (% Pd) for both types of liposomes may be above about 15% (e.g., about 24.0±1.90% for ES-targeted liposomes, and about 24.0±1.43% for control liposomes), indicating a heterogeneous distribution of particles. Finally, ES-conjugated, doxorubicin containing liposomes may have a radius of about 97.1±14.4 nm. Based on size, each of these types of liposomes may be categorized as LUVs.

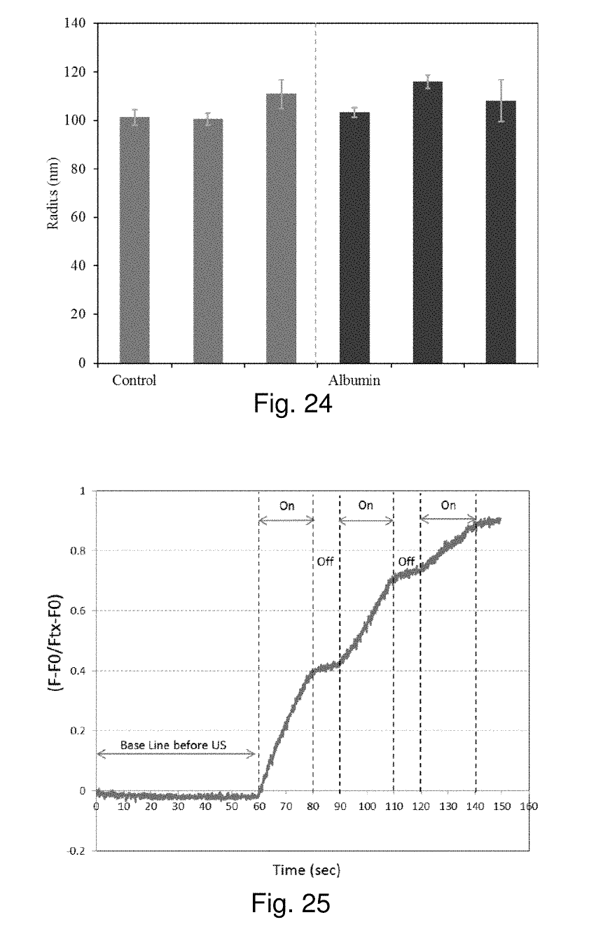

FIG. 24 illustrates the average radii for three separate batches each of DSPE-PEG-pNP control liposomes and albumin liposomes, as obtained by DLS. The average diameter for control liposomes may be about 208.52±11.50 nm. The average diameter for albumin liposomes may have be about 218.27±12.73 nm. There is no statistically significant difference between the sizes of both of these types of liposomes (p=0.38). Therefore, it may be concluded that the addition/coupling of albumin to liposomes having a diameter greater than 200 nm does not significantly increase the size of the liposomes. Again, based on size, both of these types of liposomes may be categorized as LUVs. Using other compositions and other preparation methods, it may be possible to produce liposomes of different sizes. For example, PEGylated liposomes may have an average diameter of about 111.3±59 nm, while bovine serum albumin-liposomes may have an average diameter of about 151.1±1 nm.

Nanocarriers, such as those described herein, e.g., having a particle diameter of about 200 nm, may be efficient in various DDS because their size(s) may be ideal for their accumulation via the EPR effect.

### Low Frequency US-Induced Release

Nanocarrier release using LFUS may be evaluated at a fixed input frequency (e.g., a fixed input frequency of about 20 kHz) and in PBS buffer pH of about 7.4. Release may be compared and/or optimized at various power densities (e.g., at 3 different power densities: 6.08, 6.97 and 11.83 W/cm²). Curves were obtained in triplicate (e.g., 3 batches of every liposome type). A typical LFUS-mediated release curve is shown in FIG. 25. As can be seen, before application of US, the curve begins with a baseline which measures the fluorescence level in the sample. Upon starting sonication, the fluorescence level may increase due to the calcein release. As shown in FIG. 25, ultrasound may be applied for 20 s, followed by a 10 s off period, during which a constant fluorescence level was measured due to the lack of additional calcein release. The 20 s on-10 s off cycle may be repeated until a plateau is reached (e.g., no additional calcein release can be observed).

### DSPE-PEG2000-NH2 (Control Liposomes)

So as to more effectively quantify nanocarrier release data, it may be useful to generate a release baseline and maximum (after complete liposome destruction, e.g., by adding Tx100) without any insonation. This can provide an indication and validation of the maximum release for subsequent release profiles.

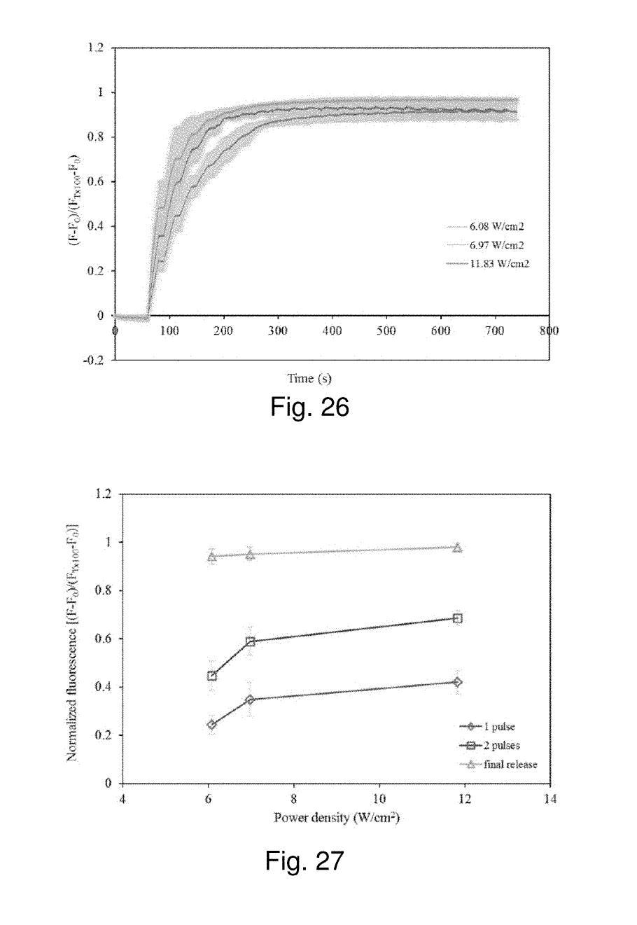

Control liposomes (e.g., two batches of control liposomes, comprising 3 replicates per batch) may be insonated (e.g., using a 20-kHz probe). Release may be compared and/or optimized at various power densities (e.g., at three power densities: 6.08, 6.97, and 11.83 W/cm²). FIG. 26 illustrates normalized, averaged calcein release profiles (showing standard of deviation) of DSPE-PEG₂₀₀₀-NH₂ control liposomes. As can be seen, the initial rate of release increased with increasing power amplitudes during about the first 200 seconds. After about 13 minutes of pulsed insonation, Tx100 may be added to lyse the liposomes and fluorescence may be measured (e.g., after an additional time, such as about 2 minutes, to reach the maximum release). As shown in FIG. 26, adding Tx100 may not cause much of a change on the measured fluorescence levels: the fluorescence level may be only slightly above the preceding plateau, which indicates that liposomes had already released most of their encapsulated calcein contents.

Initial and final rates of release may be calculated from release curves, such as the release curves shown in FIG. 26. Initial release rates may be approximated by the normalized fluorescence values obtained after the first (e.g., about 60 to 90 sec) and second (e.g., about 90 to 120 sec) pulses of US. Final release may be calculated after about 13 minutes of pulsed insonation by adding Tx100 to lyse the liposomes remaining in solution. FIG. 27 shows these results (e.g., normalized release profiles for DSPE-PEG₂₀₀₀-NH₂ control liposomes triggered by 20-kHz LFUS at 6.08, 6.97, and 11.83 W/cm²), which are also summarized in Table 5, below. The percentage of release after the first US pulse significantly increases (p<0.05) with power density: the percentage of release at 6.08 W/cm² is significantly lower (p=5.87×10⁻³) than the release obtained at 6.97 W/cm² and also significantly lower (p=1.42×10⁻⁶) than the one obtained at 11.83 W/cm². The release at 6.97 W/cm² is also significantly lower (p=0.042) than the one at 11.83 W/cm². Similar results may be obtained when comparing the release after the second US pulse: the release significantly increases (p<0.05) with increasing power density. These results show that the release may be faster for higher power densities.

The final percentage of release may be similar amongst power densities (e.g., the differences in final percentage of release between 6.08 and 6.97 W/cm² are not statistically significant (p=0.51). However, the release at lower power densities may be lower than at higher power densities (e.g., the release at 6.08 W/cm² is significantly lower (p=0.01) and the release at 6.97 W/cm² is also significantly lower (p=0.04) than the release at 11.83 W/cm², the highest power density shown in FIG. 27).

TABLE 5
Calcein release from DSPE-PEG₂₀₀₀-NH₂ liposomes triggered by
20-kHz US at the indicated power densities.
Results are average ± standard deviation of 2 liposome batches
(3 replicates each).

| Power density | Calcein release (% of normalized fluorescence) | | |
|---|---|---|---|
| (W/cm²) | Pulse #1 | Pulse #2 | Final Release |
| 6.08 | 24.36 ± 3.88 | 44.77 ± 5.96 | 94.08 ± 3.19 |
| 6.97 | 34.84 ± 6.87 | 58.90 ± 5.89 | 95.13 ± 2.79 |
| 11.83 | 41.97 ± 4.72 | 68.61 ± 2.95 | 97.93 ± 1.53 |

Based on the mechanical index described elsewhere herein, collapse/transient cavitation may be more likely to occur at low frequencies and high intensities, according to Eq. (4). Collapse/transient cavitation may also occur if the frequency is enough to counteract the effect of low power density. The MI threshold that induces a collapse/transient cavitation is about 0.7, assuming the presence/existence of free bubbles nearby. Using Eq. (4) in combination with the system parameters used to generate the data shown in Table 5, the MI values may be calculated to be 3.02, 3.23 and 4.21 for 6.08, 6.97 and 11.83 W/cm², respectively. Hence, collapse/transient cavitation thresholds were achieved at all power densities. With reference to FIG. 21, it may be concluded that sonoporation of the liposomal membrane occurs during exposure to US, resulting in an increase in measured fluorescence. The fluorescence stabilizes (e.g., ceases to increase, or flatlines) once the US is turned off; hence, it may be concluded that the membrane restores its impermeability (e.g., by healing its pores). Thus, release obtained at 20 kHz may be primarily due to collapse/transient cavitation. The power density threshold for cavitation induced by LFUS is approximately 1.2 W/cm² at 20 kHz. Additionally, Dox-encapsulating DOPE-based and DSPE-based liposomes do not disintegrate either before or after exposure to 40-kHz and 1-MHz US (as evaluated by HPLC): molecules of Dox, cholesterol and phospholipids do not disintegrate after being exposed to US, but, instead, keep their chemical structure intact. Therefore, sonoporation may be the main mechanism for drug release from liposomes triggered by US, where chemical (and/or structural) integrity of lipids remains intact/preserved after exposure to US.

Comparing the release of DPPC-chol-DSPE-NH₂ liposomes, discussed herein, to DPPC-chol-DOPE-pNP liposomes shows that the release rates from certain liposomes disclosed herein advantageously occurs at a higher rate at each power density. This may be attributed to the type of lipids used and/or to the pNP group attached. DSPE in the liposome bilayer makes it more rigid, and, therefore, more susceptible to rupture than bilayers containing DOPE instead of DSPE because the DSPE is a saturated lipid while DOPE is an unsaturated lipid. The phase of a liposomal membrane may be classified as solid-ordered (SO) (also known as crystalline, or gel phase), liquid-disordered (LD) (also known as fluid, or liquid crystalline phase), or liquid-ordered (LO). The highest liposomal permeability may achieved when the membrane is in LD phase. However, a membrane experiencing a phase transition may be even more permeable than the membrane when in the LD phase because it simultaneously exists in more than one phase. Therefore, it may be expected that chaotic change in the membrane phase will result in a greater permeability/leakage (and, therefore, more drug release from drug-containing liposomes). As a result, higher release may be obtained from saturated lipids than unsaturated lipids because saturated lipids are more rigid and will result in a phase change from SO to LD upon exposure to US. As discussed herein, the incorporation of DSPE-PEG in DOPE-based liposomes may enhance sonosensitivity (even by comparison to DOPE-based liposomes). Moreover, the optimum membrane composition to achieve the highest response to US may comprise (e.g., include at least) DOPE, DSPE-PEG, and a low level of cholesterol.

### DSPE-PEG-pNP (Control Liposomes)

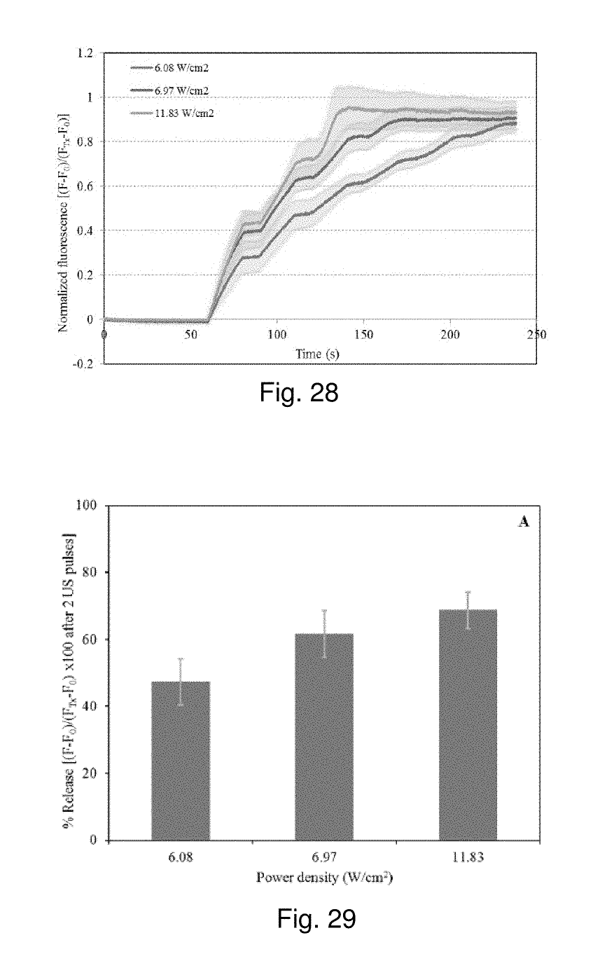

FIG. 28 shows the release curves for DSPE-PEG-pNP control liposomes at 20 kHz and at three different power densities. Error bars associated with the use of different liposome batches are shown.

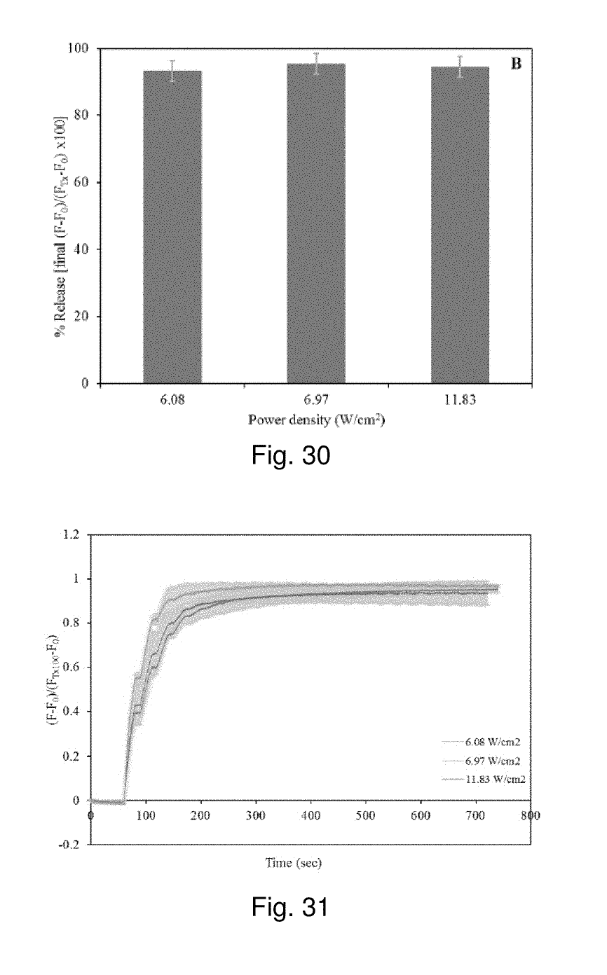

As illustrated, release can be rapid, reaching a stable value after about 150-250 s. FIG. 29 shows a comparison of initial rate of calcein release from control DSPE-PEG-pNP liposomes at different power densities, e.g., the release rate for increases in power density. FIG. 30 shows a comparison of final percentages of release from control DSPE-PEG-pNP liposomes at different power densities. As can be seen, the final percentage of release may be higher than 90% (e.g., is higher than 90% in all cases) (e.g., for all power densities). These results may be interpreted as establishing that treatment with LFUS is effective in releasing calcein from the liposomes (e.g., is effective in releasing calcein from DSPE-PEG-pNP control liposomes under the discussed parameters).

FIG. 20 illustrates that an approximate initial release rate may be determined from the calcein that was released after a second pulse of US. FIG. 30 shows that the final release may be calculated by determining the maximum fluorescence attained after the liposomes are lysed (e.g., by the addition of Tx100). Analysis of the results shows that the initial rate of release, measured as the percentage of fluorescence after the second 20 s pulse of US, may be significantly higher (p=1.10×10⁻⁶) for a power density of 6.97 W/cm² (61.67±6.93%) than for the lowest power density of 6.08 W/cm² (47.23±6.95%). The rate of release at 11.83 W/cm² (68.64±5.39%) was also significantly higher (p=8.67×10⁻³) than the one at 6.97 W/cm². By contrast, the final percentages of release showed: little to no significant difference (p=0.25) between 6.08 W/cm² (93.19±3.06%) and 6.97 W/cm² (95.36±3.16%); little to no statistical significant difference (p=0.47) between 6.08 and 11.83 W/cm² (94.55±3.12%); and little to no significant difference (p=0.14) between 6.97 and 11.83 W/cm².

For various reasons discussed herein, high release levels may be anticipated when using PEGylated liposomes. Increasing the PEG composition of liposomes up to about 5 mol % may increase the US-triggered release. In addition, DSPE-containing liposomes may also have high release levels.

Two different liposomes, (DPPC-cholesterol-[DOPE-PEG-pNP] and DPPC-cholesterol-[DSPE-PEG-pNP]) may be compared. Both of these types of liposomes contain DPPC but differ in the lipid type connected to PEG and in the amount of lipids used in their synthesis. It may be seen that DOPE-containing liposomes have a slower release rate (e.g., a much slower release rate). For example, maximum release for these liposomes may be attained at about 10 minutes of insonation, compared to about 2.5 minutes of insonation for DSPE-containing liposomes. In addition, DOPE liposomes' final release of calcein may be less than about 90% (even at the highest power density), while DSPE liposomes may have a final release of above about 90% (even at the lowest power density). Such different release profiles and/or rates may be, at least partially, explained by differences in the molecular structures of DOPE and DSPE. Unsaturated lipids, such as DOPE, have cis bonds which decrease lipids' ability to pack and transition temperature (e.g., about −16° C.), whereas DSPE lipids are saturated and can pack easily, thereby causing a high transition temperature (e.g., about 74° C.). As a result, their leakage levels (e.g., permeability) may be different. Because DSPE lipids have high transition temperatures, they are more rigid and behave like a solid material. When triggered by US, the DSPE-containing liposomes will “break” open, while the DOPE-containing liposomes, which have lower transition temperature, behave like a gel at the same/release temperatures, releasing slower. As such, DOPE-containing liposomes exposed to US are expected to release slower than DSPE-containing liposomes at the same power density and frequency (e.g., same attenuation and temperature).

### DSPE-PEG2000-N3C3Cl-ES Liposomes

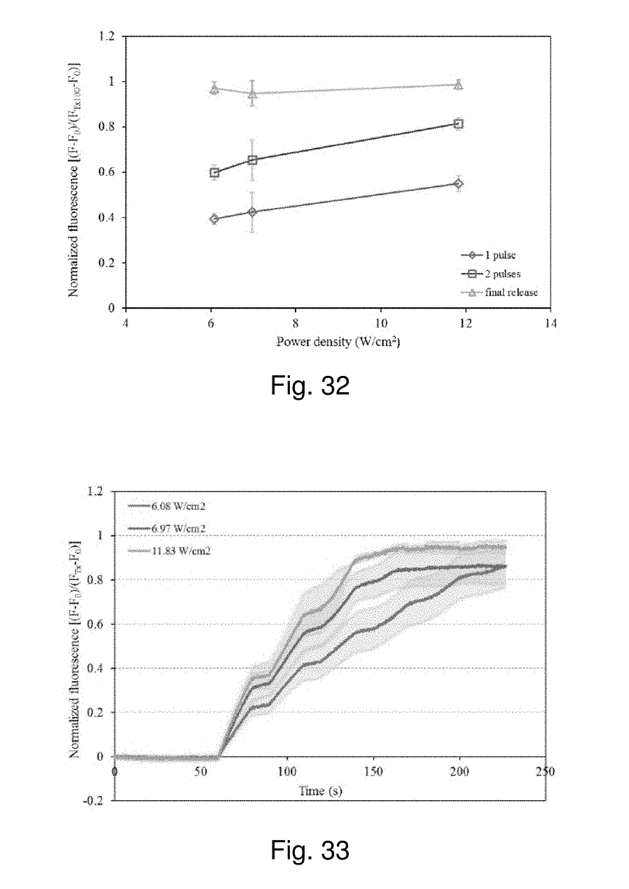

ES-targeted liposomes may be analyzed as discussed elsewhere herein (e.g., a similar analysis protocol may be used, results may be obtained using two batches of liposomes with three replicates each, and the samples may be sonicated at the same frequencies and power densities). Normalized calcein release profiles from DSPE-PEG2000-N3C3Cl-ES liposomes triggered by 20-kHz LFUS at 6.08, 6.97, and 11.83 W/cm² are shown in FIG. 31. Similarly to the discussion regarding release from control liposomes, the initial and final percentages of release may be calculated from the obtained release curves. FIG. 32 shows these results (e.g., normalized release profiles for DSPE-PEG₂₀₀₀-NH₂ control liposomes triggered by 20-kHz LFUS at 6.08, 6.97, and 11.83 W/cm²), which are also summarized in Table 6, below.

As can be seen, there is no significant difference (p>0.05) between the initial release rates (i.e., between one and two US pulses) and the final release rates for the power densities 6.08 W/cm² and 6.97 W/cm². Release after the first and second pulses is significantly higher for the highest power density, i.e., 11.83 W/cm², when compared to release at 6.08 W/cm² (p=8.85×10⁻⁶ and p=3.13×10⁻⁷, respectively) and release at 6.97 W/cm² (p=4.39×10⁻³ and p=1.10×10⁻³, respectively). There were no statistically significant differences (p>0.05) of the final release percent for the three power densities studied. Based on the calculated MI values discussed elsewhere herein, cavitation may be the main mechanism of release for these liposomes.

TABLE 6
Calcein release from DSPE-PEG₂₀₀₀-N₃C₃Cl-ES liposomes triggered by
the 20-kHz probe at the indicated densities.
Results are average ± standard deviation of 2 liposome batches
(3 replicates each).

| Power density | Calcein release (% of normalized fluorescence) | | |
|---|---|---|---|
| (W/cm²) | Pulse #1 | Pulse #2 | Final Release |
| 6.08 | 24.36 ± 3.88 | 44.77 ± 5.96 | 94.08 ± 3.19 |
| 6.97 | 34.84 ± 6.87 | 58.90 ± 5.89 | 95.13 ± 2.79 |
| 11.83 | 41.97 ± 4.72 | 68.61 ± 2.95 | 97.93 ± 1.53 |

US, as a trigger, may also be used to stimulate release from targeted micelles. Initial release percentages for micelles may be higher because the structure of these drug delivery carriers is composed of a single layer, not a bi-layer as is the case with liposomes. About 1-2 seconds may be sufficient to cause approximately 8% release from some micelles (e.g., of a drug (e.g., doxorubicin) from Pluronic® micelles (e.g., Pluronic® P105 micelles) (e.g., at about 6 W/cm², and 70-kHz US). Other parameters, such as those disclosed herein, may be used to stimulate micelles. US release from micelles may also show a statistically significant difference between targeted and non-targeted micelles (e.g., targeted micelles may show a higher release percentage). There are at least three main differences between acoustic micellar and liposomal release: first, 70-kHz US may be used in micellar stimulation, while 20-kHz US may be used in liposomal stimulation; second, the encapsulated substance may be different between micelles and liposomes (e.g., an actual chemotherapeutic drug may be encapsulated inside the micelles whereas a model drug may be encapsulated inside liposomes); and third, the targeting moiety conjugated to micelles may be different than that conjugated to liposomes (e.g., folic acid may be conjugated to micelles and estrone may be used to induce receptor mediated endocytosis via the use of targeted liposomes). Regardless, design criteria and principles employed by each of the two nanocarriers may be relevant to the other: both 20 kHz and 70 kHz are considered to be in the low acoustic frequency range; Dox and calcein have similar molecular weights and aromatic rings; and both folic acid and estrone are low molecular weight targeting moieties. Therefore, the information presented herein may have applicability to targeted micelles as well as targeted liposomes. One germane difference between micelles and liposomes is that upon the termination of sonication, originally-encapsulated drugs may be re-encapsulated inside micelles but are not re-encapsulated inside liposomes.

### DSPE-PEG-Albumin Liposomes

FIG. 33 illustrates calcein release curves from albumin-targeted liposomes (e.g., DSPE-PEG-albumin liposomes) triggered by 20 kHz US (e.g., 20 s on 10 s off), at the three different power densities mentioned previously. Error bars associated with the use of different liposome batches are also illustrated.

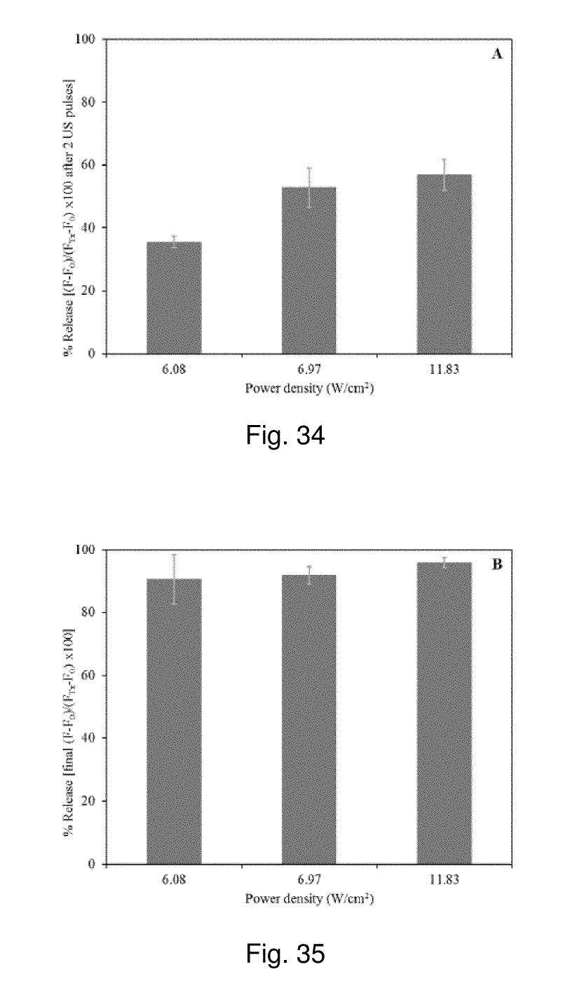

Similar to the release observed in control liposomes discussed herein, such liposomes may observe fast release and reach a stable value after about 150-250 s. FIG. 34 shows a comparison of the initial release rate of calcein from DSPE-PEG-albumin liposomes at different power densities and FIG. 35 shows a comparison of the final release percentage of calcein from DSPE-PEG-albumin liposomes at different power densities. As illustrated in FIG. 34, the release rate increases with increasing power density, and, as shown in FIG. 35, the final percentage of release is higher than about 90% for each power density shown.

The initial rate of release (e.g., the percentage of fluorescence after the second 20 s pulse of US) was significantly lower (p=8.79×10⁻⁶) for a power density of 6.08 W/cm² (35.57±1.78%), when compared to 6.97 W/cm² (52.83±6.26%) and also significantly lower (p=1.08×10⁻⁶) than that which may be obtained at 11.83 W/cm² (56.87±5.00%). The initial rate of release at 11.83 W/cm² (56.87±5.00%) was not significantly different (p=0.07) from that which may be obtained at 6.97 W/cm², unlike of the release observed from control liposomes. The final percentages of release show no significant difference (p=0.66) between 6.08 W/cm² (90.63±7.88%) and 6.97 W/cm² (91.94±2.77%). Also no significant difference (p=0.61) between 6.08 and 11.83 W/cm² (95.86±1.49%), and no significant difference (p=0.79) between the 6.97 and 11.83 W/cm² power densities were observed.

### Comparison Between ES-Conjugated and Control Liposomes

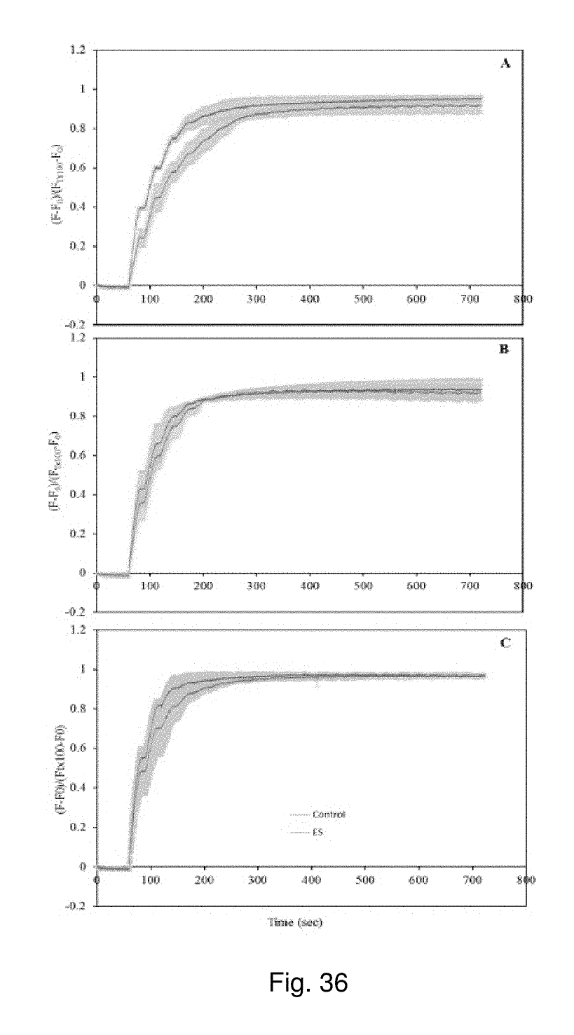

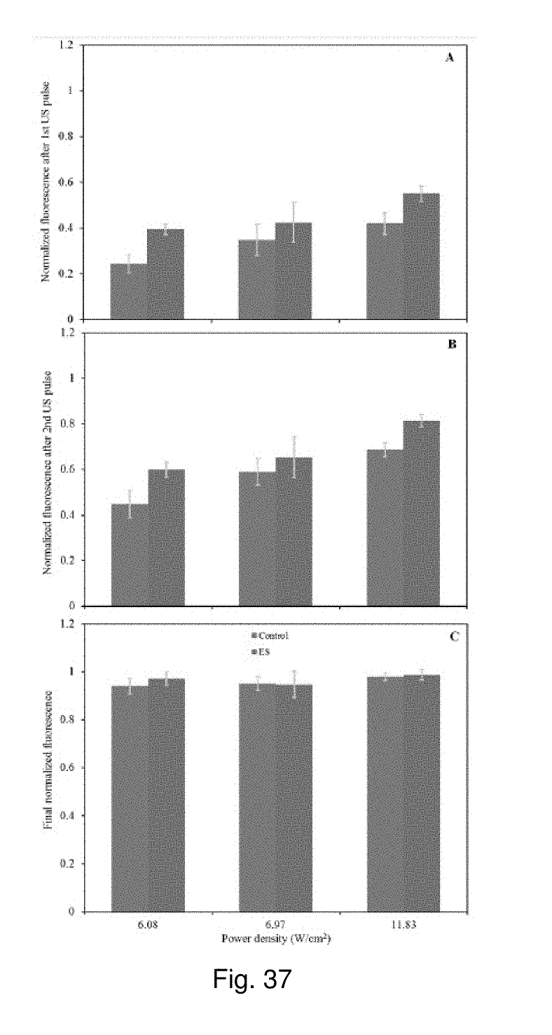

The performance of ES-conjugated liposomes may be evaluated by comparing the release of ES-conjugated liposomes against the release of control liposomes, disclosed herein (e.g., by evaluating the final release percent, as well as the release achieved after the first two pulses). FIG. 36 illustrates various comparisons between the normalized calcein release profiles from DSPE-PEG₂₀₀₀-N₃C₃Cl-ES and DSPE-PEG₂₀₀₀-NH₂ liposomes: FIG. 36A shows the release profiles when the liposomes are triggered by 20-kHZ LFUS at 6.08 W/cm²; FIG. 36B shows the release profiles when the liposomes are triggered by 20-kHZ LFUS at 6.97 W/cm²; and FIG. 36C shows the release profiles when the liposomes are triggered by 20-kHZ LFUS at 11.83 W/cm². FIG. 37 illustrates various comparisons of calcein release from DSPE-PEG₂₀₀₀-N₃C₃Cl-ES and DSPE-PEG₂₀₀₀-NH₂ liposomes triggered by 20-kHz at 6.08, 6.97, and 11.83 W/cm²: FIG. 37A shows release after the first ultrasound pulse; FIG. 37B shows release after the second ultrasound pulse, and FIG. 37C shows the final release. There are no statistically significant differences (p>0.05) between the final releases from targeted and non-targeted liposomes, at any power density. The targeted liposomes show a significantly higher release than non-targeted liposomes at both 6.08 W/cm² and 11.83 W/cm², after the first (p=1.36×10⁻⁶ and p=6.63×10⁻⁵, respectively) and the second US pulses (p=6.64×10⁻⁵ and p=2.54×10⁻⁶, respectively).

Estrone, which is similar in at least one respect to cholesterol, is insoluble in aqueous solutions. Therefore, some estrone molecules that are anchored to the surface of estrone-targeted liposomes (e.g., DSPE-PEG₂₀₀₀-N₃C₃Cl-ES liposomes) may attempt to escape the surrounding aqueous medium (e.g., the aqueous PBS buffer, or blood) by incorporating themselves into the bilayer membrane. Such inclusion of estrone molecules into a phospholipid membrane may cause disorder and defects in the liposome's membrane, which may ultimately facilitate release as sonoporation occurs.

### Comparison Between Targeted Micelles and Targeted Liposomes

Like the US mediated release described with respect to liposomes, acoustic power may also be employed to trigger the release of chemotherapeutics from folated (folic acid targeted) micelles. Initial drug release percentages from targeted micelles may be higher than that observed with respect to targeted liposomes, in part because their structure is composed of a single layer, as opposed to the phospholipid bilayer comprising the liposomes. For example, two seconds may be sufficient to cause approximately 8% release of a drug from micelles (e.g., of Dox from Pluronic® P105 micelles) at about 6 W/cm², using low-frequency ultrasound.

### Comparison Between Albumin-Conjugated and Control Liposomes

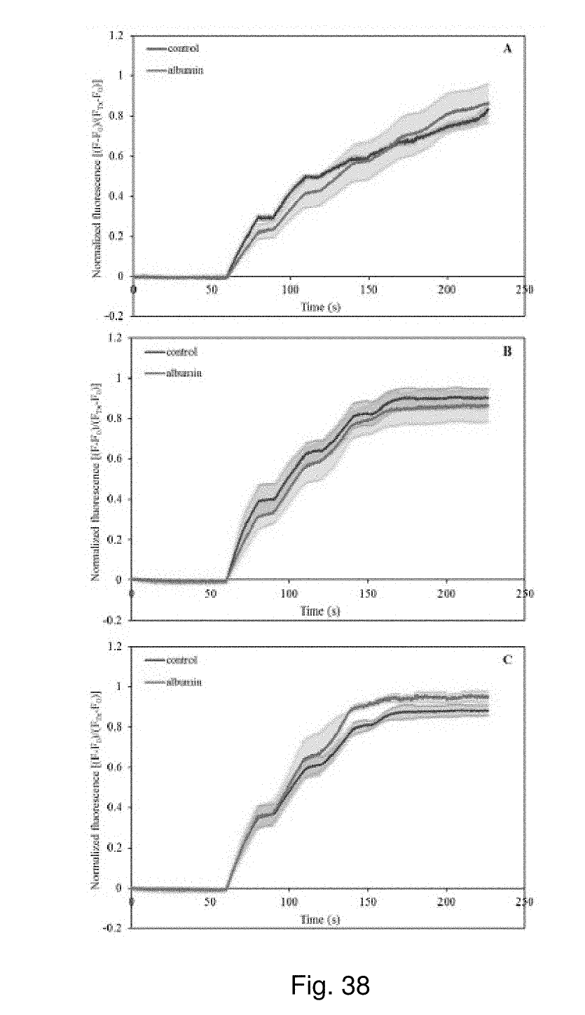

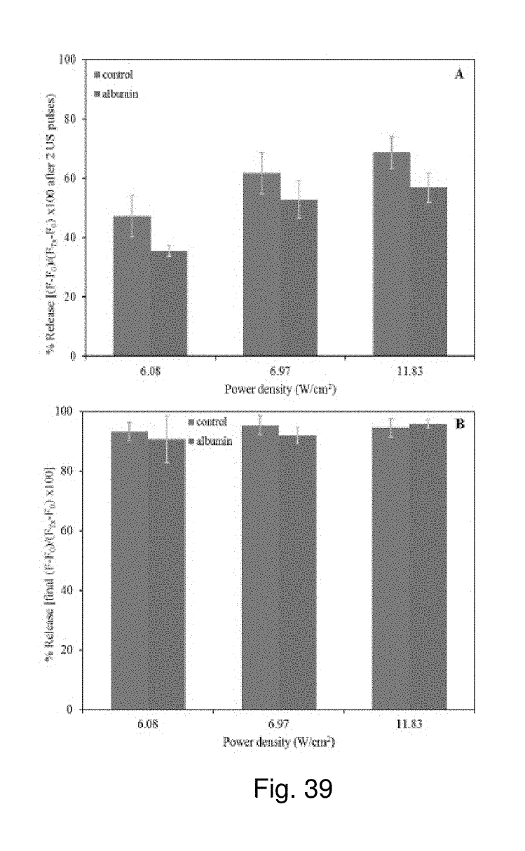

Comparison of initial release rates (shown in FIG. 38) and final percentages of release (shown in FIG. 39) of control and albumin liposomes may be performed for each power density to evaluate the performance of albumin liposomes. FIG. 38 illustrates comparisons of the release rate, evaluated by the fluorescence after the second US pulse, for the control liposomes as disclosed herein and albumin liposomes: FIG. 38A shows the comparison of the release curves at 6.08 W/cm²; FIG. 38B shows the comparison of the release curves at 6.97 W/cm²; and FIG. 38C shows the comparison of the release curves at 11.83 W/cm². FIG. 39 illustrates comparisons of the rates of calcein release from control and DSPE-PEG-albumin liposomes at different power densities: FIG. 39A shows release after the first ultrasound pulse; and FIG. 39B shows release after the second ultrasound pulse.

As illustrated by FIG. 39A, for the same US power density, the release from control liposomes is significantly faster than for the albumin liposomes (p=6.40×10⁻³ for 6.08 W/cm², p=9.51×10⁻⁴ for 6.97 W/cm², and p=4.24×10⁻⁴ for 11.83 W/cm²). FIG. 39B illustrates a comparison of the final release from targeted and non-targeted liposomes showing no significant differences at 6.08 W/cm² (p=0.36) and at 11.83 W/cm² (p=0.59), while the final release at 6.97 W/cm² is significantly higher (p=1.68×10⁻²) for control liposomes. Results suggest that the conjugation of an albumin moiety to the surface of the liposomes may change their acoustic properties, for example, by increasing their stability and making them less susceptible to US.

Since albumin is considered a natural nanocarrier (e.g., it can carry paclitaxel, as described in herein), some albumin moieties, when attached to PEG, may capture some of the released calcein and decrease its concentration in the solution. Consequently, the fluorescence level may decrease. As such, the apparent release rate of albumin liposomes may be lower than that from control PEGylated-only liposomes.

At least some calcein release is due (e.g., mainly due) to the mechanical effects of US (e.g., cavitation). As the US frequency decreases, the threshold for transient cavitation may also decrease. Therefore, at 20 kHz, using a 25 mm sonication probe, the mechanical index MI may be calculated as 3.02, 3.23, 4.21 for 6.08, 6.97, 11.83 w/cm² power densities, respectively. The values of MI indicate that the cavitation is transient because all values are above the transient threshold, e.g., about 0.7. As a result of bubble collapse, any one or more of the following may be observed: intense local heating, shock wave, micro streaming, and shear forces. Local heating may cause hot spots with very high temperatures and pressures. Such localized hyperthermia may tend to increase the permeability of the liposomal lipid layer; hence, hyperthermia may increase localized calcein release.

### High Frequency US Release

As described herein, HFUS may be used to trigger release of calcein from liposomes, e.g., control liposomes. High frequency US (HFUS) is a term used for US waves with frequencies higher than about 1 MHz. Release profiles for various liposomes were evaluated using two high frequencies: 1.07 MHz and 3.24 MHz, at two power densities (10.5 and 50.2 W/cm2) for the former frequency, and one power density (˜173 W/cm2) for the latter frequency. Of course, any of the power densities discussed herein may be used with any of the ultrasound frequencies discussed herein.

### DSPE-PEG2000-NH2 (Control Liposomes)

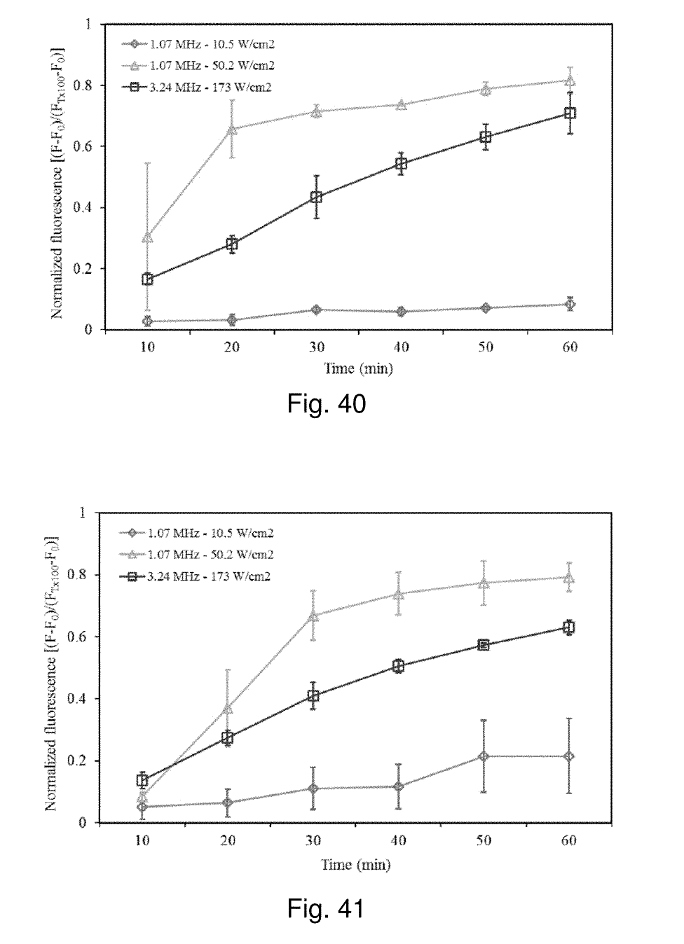

Control liposomes (e.g., two or more batches) may be sonicated at each power density (e.g., in at least three technical replicates). FIG. 40 illustrates normalized calcein release profiles from DSPE-PEG₂₀₀₀-NH₂ liposomes, triggered by: 1.07 MHz HFUS at a power density of 10.5 W/cm²; 1.07 MHz HFUS at a power density of 50.2 W/cm²; and 3.24 MHz HFUS at a power density of 173 W/cm².

With continued reference to FIG. 40, when using 1.07 MHz HFUS, the release significantly increased (p<0.05) with power density at each time point, except at 10 min. The release using a power density of 10.5 W/cm² was much lower than that using a power density of 50.2 W/cm², and it showed only a small increase with time, ultimately reaching a final release of 8.45% after 60 min of insonation. The release using a power density of 50.2 W/cm² shows a high rate of increase during the first 20 minutes of insonation, after which the increase continues at a slower rate. At this power density, the final release achieved after 60 min of insonation was 81.67%, which is approximately 10 times more than that achieved using a power density of only 10.5 W/cm². The standard deviation for 1.07 MHz HFUS at a power density of 50.2 W/cm² is large at the first 10 minutes of insonation. This may be due to random attenuation of US waves as bubbles formed on the external surface of the beaker holding the sample.

The release profile at the higher HFUS frequency of 3.24 MHz shows a more sustained pattern of linear increase over time, but the calcein release after each and every 10-minute insonation period is still significantly less than that achieved when using the higher power density at 1.07 MHz. Table 7 summarizes the average percentage of release for all power densities at the aforementioned frequencies.

TABLE 7
Calcein release from DSPE-PEG₂₀₀₀-NH₂ liposomes triggered by the 1.07
and 3.24-MHz probes at 10.5, 50.2, and 173 W/cm² power densities.
Results are average of 2 liposome batches (3 replicates each).

Calcein release (% normalized fluorescence)

| | |
|---|---|
| 1.07 MHz | 3.24 MHz |

| Time (min) | 10.5 W/cm² | 50.2 W/cm² | ~173 W/cm² |
|---|---|---|---|
| 10 | 2.73 | 30.54 | 16.60 |
| 20 | 3.20 | 65.79 | 28.02 |
| 30 | 6.55 | 71.58 | 43.42 |
| 40 | 5.93 | 73.68 | 54.33 |
| 50 | 7.16 | 78.95 | 63.14 |
| 60 | 8.45 | 81.67 | 70.92 |

The mechanical indices were determined in a similar way as described elsewhere herein. The calculated MI values were 0.54, 1.19 and 1.27 for 10.5, 50.2 and 173 W/cm², respectively. At 10.5 W/cm², only about 8.45% calcein release was achieved after one hour of insonation which implies that collapse cavitation was not successfully achieved (when compared to releases at the other two power densities). This is supported by the fact that the calculated MI at the power density of 10.5 W/cm² is below the threshold value for collapse cavitation (MI=0.54<0.7); hence, release might have been induced by the effect of hyperthermia and/or stable cavitation. The mechanism of release at both power densities of 50.2 and 173 W/cm² is likely collapse/transient cavitation, since the corresponding MI values exceed the threshold value needed to induce cavitation. Release for the power density of 50.2 W/cm² is higher than the release for the power density of 173 W/cm² because higher frequency was used for the latter power density. This is consistent with the fact that attenuation of US waves increases as frequency increases. Thus, it may be necessary to increase power density (to compensate for energy losses, such as heat dissipation) to achieve a higher release using HFUS at a frequency of 3.24 MHz. As can be seen, cavitation events may be more easily induced using LFUS than HFUS. Therefore, improved release (e.g., higher release) may advantageously be achieved using LFUS, by comparison to HFUS. Such behavior may be due to the fact that cavitation may be more likely to occur when bubbles are given enough time to grow and collapse. The time interval for the negative peak pressure which may be achieved using LFUS is sufficient for nucleation. Therefore, US-mediated drug release from nanocarriers (e.g., UD-mediated liposome release) may be improved using LFUS.

Certain liposomes described herein are DPPC-based (i.e., the major lipid constituent), and comprise a small fraction of DSPE-PEG and cholesterol. The transition temperature of DPPC is about 41.5° C., and the transition temperature of DSPE is even higher (e.g., about 74° C.). However, inclusion of cholesterol may counteract some effects of DSPE (e.g., the contribution of DSPE towards a potentially higher transition temperature) by its ability to lower the transition temperature of saturated lipids. Therefore, hyperthermia may be an additional mechanism membrane permeation and subsequent release of calcein, especially at 3.24 MHz because the attenuation of US waves (and conversion to heat) increases with increasing frequency. Another mechanism that may contribute to calcein release is “acoustic streaming,” which has a mechanical effect arising from the partial absorption of US energy by the fluid. This energy is translated then to a convective flow in the direction of US propagation, thereby inducing mixing and the collision of nanoparticles that may ultimately rupture the membrane and release encapsulated drug (e.g., calcein).

### DSPE-PEG2000-N3C3Cl-ES

Liposomes targeted with ES-N₃C₃Cl₂ conjugate (e.g., two batches having 3 replicates each) may be evaluated for drug release at the same two frequencies (e.g., 1.07 and 3.24 MHz) and power densities. Insonation may be conducted for about 60 minutes in approximately 10-min intervals, after which the fluorescence may be recorded. FIG. 41 illustrates normalized calcein release profiles from DSPE-PEG₂₀₀₀-N₃C₃Cl-ES liposomes triggered by: 1.07 MHz HFUS at a power density of 10.5 W/cm²; 1.07 MHz HFUS at a power density of 50.2 W/cm²; and 3.24 MHz HFUS at a power density of 173 W/cm². The release profiles at 1.07 MHz are similar to those observed in connection with control liposomes, discussed herein (see FIG. 40) Calcein release increases as the power density is increased from 10.5 to 50.2 W/cm² at each time interval. At a power density of 50.2 W/cm², the calcein release increases at a higher rate during the first 30 minutes of insonation compared to the rate of release during the last 30 minutes. Similar behavior may be observed in connection with control liposomes, as illustrated in FIG. 40. The release at a power density of 10.5 W/cm² shows only a slight increase over time and reaches a final release of about 21.51% compared to a final release of about 79.31% at the higher power density of 50.2 W/cm². For all the time points after the first 10 min, the release at the power density of 10.5 W/cm² was significantly lower (p<0.05) than the release at the power density of 50.2 W/cm².

Release at the higher frequency of 3.24 MHz (at a power density of 173 W/cm²) exhibits a substantially linear increase over time and achieves a final release after one hour of about 63.02%, which is significantly lower than that achieved using the 1.07 MHz HFUS at a power density of 50.2 W/cm². The mechanism of release may be hyperthermia and/or stable cavitation at the power density of 10.5 W/cm² and collapse/transient cavitation at the power densities of 50.2 and 173 W/cm², for reasons similar to those discussed elsewhere herein (e.g., in connection with FIG. 40). Additionally, acoustic streaming and hyperthermia may also contribute to release at for the 3.24 MHz HFUS. As can be seen, except for the first 10 minutes of insonation, the release increases significantly with power density. Table 8 summarizes the average percentage of release for all power densities and frequencies.

TABLE 8
Calcein release of DSPE-PEG₂₀₀₀-N₃C₃Cl-ES liposomes triggered by the
1.07 and 3.24-MHz probes at 10.5, 50.2, and 173 W/cm² power densities.
Results are average of 2 liposome batches (3 replicates each).

Calcein release (% normalized fluorescence)

| | |
|---|---|
| 1.07 MHz | 3.24 MHz |

| Time (min) | 10.5 W/cm² | 50.2 W/cm² | ~173 W/cm² |
|---|---|---|---|
| 10 | 5.18 | 8.5 | 13.60 |
| 20 | 6.41 | 36.99 | 27.43 |
| 30 | 11.07 | 66.81 | 40.96 |
| 40 | 11.71 | 73.95 | 50.49 |
| 50 | 21.44 | 77.44 | 57.26 |
| 60 | 21.51 | 79.31 | 63.02 |

### Comparison Between ES-Conjugated and Control Liposomes

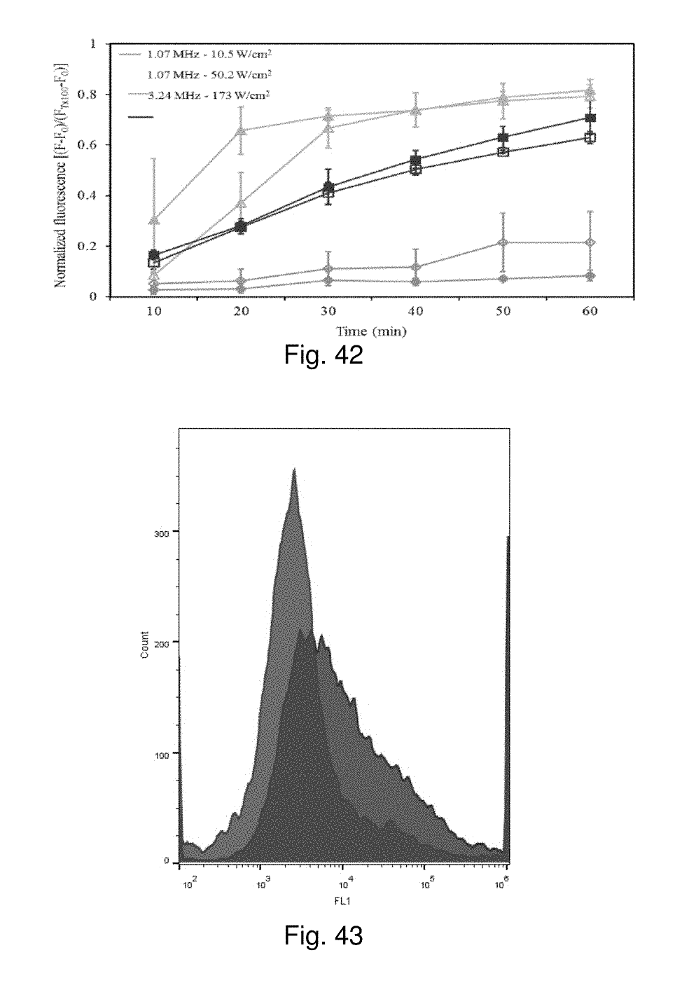

The performance of ES-conjugated liposomes may be evaluated by comparing the release of the ES-conjugated against the release of control liposomes, disclosed here (e.g., comparing the release at each frequency and power density). FIG. 42 illustrates such a comparison of normalized calcein release profiles from DSPE-PEG₂₀₀₀-N₃C₃Cl-ES liposomes and DSPE-PEG₂₀₀₀-NH₂ liposomes, each triggered by: 1.07 MHz HFUS at a power density of 10.5 W/cm²; 1.07 MHz HFUS at a power density of 50.2 W/cm²; and 3.24 MHz HFUS at a power density of 173 W/cm². Results are also presented in Table 9, below. As can be seen, there are no statistically significant differences between the two types of liposomes except for: 1.07 MHz HFUS at a power density of 10.5 W/cm² (where the release from estrone liposomes is significantly higher (p<0.05) at 50 and 60 min); and 1.07 MHz HFUS at a power density of 50.2 W/cm² (where the release from estrone liposomes is significantly higher 20 min. As such, it may be concluded that any effect of the targeting moiety (ES-C₃N₃Cl₂) on the liposomes' release profile is negligible, i.e., this targeting moiety does not interfere with release at HFUS.

TABLE 9
T-test with unequal variances including the p-values to compare calcein
release from ES-conjugated versus control liposomes triggered by the
1.07 and 3.24-MHz probes at 10.5, 50.2, and 173 W/cm² power densities

| | |
|---|---|
| 1.07 MHz | 3.24 MHz |

| Time (min) | 10.5 W/cm² | 50.2 W/cm² | 173 W/cm² |
|---|---|---|---|
| 10 | 0.2465 | 0.2542 | 0.1903 |
| 20 | 0.1702 | 0.0169 | 0.8012 |
| 30 | 0.1661 | 0.3244 | 0.6374 |
| 40 | 0.1087 | 0.9424 | 0.2014 |
| 50 | 0.0293 | 0.7101 | 0.1329 |
| 60 | 0.0452 | 0.5198 | 0.1709 |

### Evaluation of Drug Delivery Systems In Vitro

The MCF-7 (ER-positive human breast adenocarcinoma) and MDA-MB-231 (ER-negative human breast adenocarcinoma) cell lines may be used to assess the performance of one or more of the nanoparticles discussed herein. Both of these cell lines may be cultured in RPMI medium supplemented with about 10% heat inactivated fetal bovine serum (FBS) and about 1% penicillin-streptomycin. The cell cultures may be maintained at about 37° C. in a humidified atmosphere with about 5% CO₂. Twenty-four hours before an uptake studies, exponentially growing cells may be harvested with about 3 mL of trypsin and about 3×10⁵ cell/mL of growth medium seeded in 6-well plates so as to reach confluency at/by the time of the experiment.

### Cellular Uptake of ES-Conjugated Calcein Liposomes Vs Control Liposomes

In order to evaluate the effect of the presence of ERs on the cellular uptake of the ES-conjugated liposomes, flow cytometry may be used to measure the cellular uptake of calcein by an ER-positive (MCF-7) and ER-negative (MDA-MB-231) human breast adenocarcinoma cell line. Optimization of evaluation may be achieved by using and examining several concentrations of liposomes and fluorophore, including, but not limited to, about 200 μM of liposomes (concentration estimated by the amount of the major lipid incorporated by the nanoparticle, DPPC, using the Stewart assay) and about 30-50 mM of calcein.

Liposomes may be incubated for about 30 minutes with both cell lines and flow cytometry analysis may be performed to evaluate the efficacy of ES-conjugated liposomes in actively targeting the ER-positive breast cancer cell line.

The cellular uptake of calcein from ES-conjugated liposomes is statistically higher in MCF-7 than the uptake in MDA-MB-231 (p=0.000698). The average geometric mean of the peak fluorescence for ES-conjugated liposomes in MCF-7 was 13,300±6,208 while, it was 3,447±629 in MDA-MB-231. FIG. 43 shows representative flow cytometry histograms that compare uptake in MCF-7 (the curve on the right) and in MDA-MB-231 (the curve on the left). As can be seen, the prevalence of ligand-mediated endocytosis may be regulated by the binding of estrone on the endoplasmic reticula. From these results, it may be concluded that the estrone moiety confers upon the carrier the ability to recognize and confer site specificity.

To confirm this pathway's role in the accumulation of ES-targeted liposomes in ER-positive breast cancer cells, targeted and non-targeted liposomes may be incubated with ER-negative (MDA-MB-231) cells. The geometric means of the average cellular calcein uptake for ES-conjugated targeted and non-targeted liposomes were 2,351±1,166 and 2,294±285, respectively. These values are not statistically different (p=0.47024) which indicates that the accumulation of calcein in ER-negative cell lines is not affected by the presence of the targeting moiety (ES) on the liposome's surface—in short, estrone functionalized liposomes are effective in selectively targeting estrogen expressing cells.

These results confirm the ability of the estrone moiety to specifically target ER-positive breast cancer cells, thus facilitating cellular uptake and providing evidence of the potential of using estrone-conjugated nanocarriers as drug delivery vehicles in targeting certain types of breast cancer.

### Cellular Uptake of ES-Conjugated Calcein Liposomes After Ultrasound Exposure

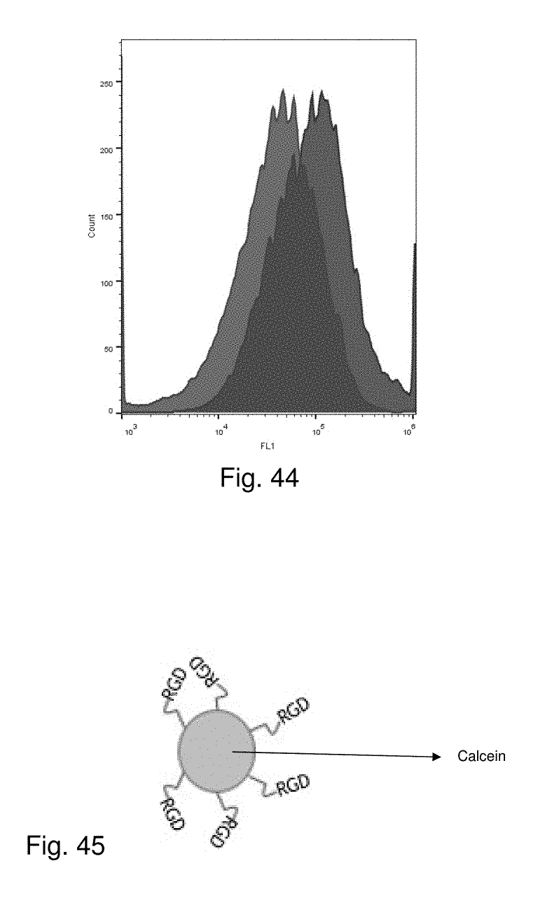

In order to evaluate the effect of both estrone functionalization and ultrasound application, MCF-7 cell plates incubated with ES-conjugated calcein liposomes may be sonicated while floating in a 40-kHz water bath. It may be seen that sonication significantly increases the intracellular uptake of calcein (p=0.001026). An example of the flow cytometry histograms of MCF-7 cells incubated with these targeted nanoparticles before and after sonication are shown in FIG. 44. The average geometric mean of the peak fluorescence for the sonicated and control cells were 55,148±14,625 and 36,357±11,409, respectively, which represents an increase of 70% in calcein accumulation in MCF-7 cells after ultrasound exposure.

Several mechanisms could play a part in causing the US-induced release and uptake observed. Regarding the sonoporation of cell membranes, US has been implemented to induce the formation of micropores in the cell membrane. As a result, sonoporation improves the uptake of large molecules by human cells. The effect of sonoporation on the cell accumulation of small molecules like calcein or doxorubicin, however, is unclear. The cell retention of small molecules may be compromised by the presence of micropores. The drug concentration inside the sonicated cells may be higher than the extracellular concentration. Finally, the poration of the cell membrane can lead to the leaking of free drug if the molecule is not already attached to a cell structure like the DNA to satisfy the thermodynamic equilibrium established across the cell membrane.

Liposomes Modified with RGD

RGD-functionalized liposomes may be prepared substantially as discussed above with respect to estrone functionalized liposomes. RGD-modified liposomes may be used to selectively target liver cancer, among others. RGD may first be attached to a lipid (e.g., DSPE-PEG-NH₂). Then, liposomes may be formed using this modified lipid. As shown in FIG. 45, calcein may be encapsulated in the liposomes as a model drug to study the release from those liposomes using ultrasound. Of course, any of a number of other drugs, such as discussed elsewhere herein may be incorporated, including, but not limited to doxorubicin.

Similar to the methods of manufacture discussed above, DSPE-PEG-RGD may be formed by, first, reacting RGD with cyanuric chloride at about 0° C. for about 5 hours, and second, reacting the RGD-cyanuric chloride conjugate with DSPE-PEG-NH2 for an extended time, e.g., overnight, at about room temperature.

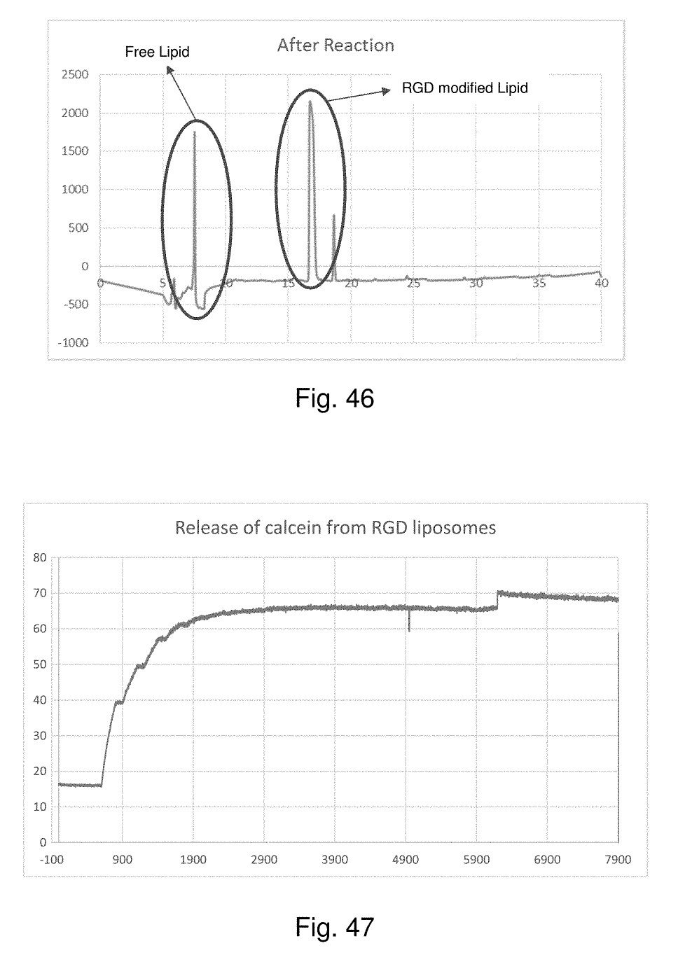

Successful conjugation and attachment of RGD to the lipid may be accomplished using high pressure liquid chromatography (HPLC). An HPLC scan of RGC conjugated with DSPE-PEG-NH₂ is shown in FIG. 46. The HPLC scan reveals a clear difference between the free lipid, DSPE-PEG-NH₂, and the RGD-lipid conjugate.

Much as described elsewhere herein, pulsated ultrasound waves may be used to evaluate the release of calcein, or any other drug, from the RGD-modified liposomes. For example, FIG. 47 shows a graph of ultrasound pulse versus fluorescence. As can be seen, the fluorescence increases rapidly in the first few ultrasound pulses. Finally, the fluorescence plateaus after only 4-5 ultrasound pulses indicating a maximum release.

RGD-functionalized liposomes appear to be similar in sonosensitivity to the above-discussed estrone-functionalized liposomes. Other ligands may be used to functionalized liposomes according to one or more of the methods disclosed herein. For example, liposomes may be advantageously functionalized using any of estrone, RGD, transferrin, and/or plasminogen. Of course, one of ordinary skill in the art will readily understand that any of a number of other ligands may be used to functionalize liposomes as disclosed herein, including, but not limited to, antibodies (e.g., monoclonal antibodies, SM 5-1, Trastuzumab, HER2, peripheral antibodies, antibodies for EGFR, etc.), peptides, folates, aptamers, hyaluronate, and transferrin.

Drug delivery systems include nanoparticles designed to transport cytotoxic drugs specifically to tumor sites to spare surrounding tissues from potentially adverse effects associated with conventional chemotherapy. As shown herein, liposomes are a type of nanocarrier that may be used to target tumors either by the EPR effect, or more selectively by attaching a targeting moiety that matches a receptor (e.g., a receptor present on the surface of cancer cells). They can encapsulate the drug and can travel to the cancer site with less clearance by the immune system, since their structure resembles that of cells. Once they reach the tumor site, they can release their encapsulated content (e.g., an anti-neoplastic) via at least three mechanisms: passive, active, and triggered targeting. Once a nanocarrier, such as a liposome, reaches a target site, it may benefit from (e.g., require) a triggering stimulus to release its load. As has been shown, DPPC-based liposomes targeted with estrone-cyanuric conjugate and encapsulating a drug (e.g., a model drug calcein) may be used as part of a treatment regimen for various conditions (including, but not limited to, breast cancer).

As discussed herein liposomes may be prepared according to various methods, including but not limited to the lipid film hydration method. Liposome uniformity and size may be evaluated using DLS. At least one of the liposomes disclosed herein may be categorized as LUVs. At least some liposomes disclosed herein (e.g., control and ES-targeted liposomes) exhibit no statistical difference in final calcein release when using LFUS (e.g., when comparing both types of liposomes at the same power amplitude/density). However, certain liposomes disclosed herein (e.g., control and ES-targeted liposomes) have significantly different release profiles during the first two US pulses at 6.08 and 11.83 W/cm² power densities (e.g., estrone-conjugated liposomes showed a significantly higher release in comparison to non-targeted liposomes). Both targeted and control liposomes may have significantly higher release rates with increasing power densities during the first two pulses. However, control liposomes may exhibit a different final release response to power density in comparison to estrone-conjugated liposomes. Certain liposomes disclosed herein (e.g., including ES-conjugated liposomes and control liposomes) may show a significant increase in release with increasing power densities where subjected to HFUS. Although, there may not be any statistically significant difference between the two types of liposomes when compared at a given/certain power density and frequency. Higher frequencies of ultrasound than those explicitly discussed herein may be used as they may be more appealing to drug delivery researchers and clinicians since such waves can be easily focused (e.g., HFUS may be preferably to LFUS due, at least in part to its focusing capabilities). Additionally, certain estrone molecules discussed herein were conjugated to liposomes using PEG spacers attached to the surface of the liposomes; consequently these molecules will escape the aqueous medium by incorporating themselves in the membrane bilayer. Such behavior may affect the ability of the targeted liposomes to effectively target their receptors. The PEG spacer length may be increased beyond that explicitly disclosed here in order to hinder the estrone from folding back to/into the lipid bilayer.

As discussed herein, albumin-modified liposomes may be prepared from control liposomes, and then reacted with albumin under specified conditions to chemically attach the protein to their surface. These albumin-modified liposomes may attain a 20-kHz ultrasound-induced release value of about 90% after about 150-250 s for all power densities discussed herein. The release rate from these albumin-targeted liposomes generally increases with increasing power densities. The release from non-targeted (e.g., control) liposomes may be significantly faster than for albumin liposomes, at the same power density. These albumin-targeted liposomes may have application in the treatment of cancer cells overexpressing albumin receptors (e.g., prostate cancer cell line DU-145).

The foregoing description and examples has been set forth merely to illustrate the disclosure and are not intended as being limiting. Each of the disclosed aspects and embodiments of the present disclosure may be considered individually or in combination with other aspects, embodiments, and variations of the disclosure. In addition, unless otherwise specified, none of the steps of the methods of the present disclosure are confined to any particular order of performance. Modifications of the disclosed embodiments incorporating the spirit and substance of the disclosure may occur to persons skilled in the art and such modifications are within the scope of the present disclosure. Furthermore, all references cited herein are incorporated by reference in their entirety.

Terms of orientation used herein, such as “top,” “bottom,” “horizontal,” “vertical,” “longitudinal,” “lateral,” and “end” are used in the context of the illustrated embodiment. However, the present disclosure should not be limited to the illustrated orientation. Indeed, other orientations are possible and are within the scope of this disclosure. Terms relating to circular shapes as used herein, such as diameter or radius, should be understood not to require perfect circular structures, but rather should be applied to any suitable structure with a cross-sectional region that can be measured from side-to-side. Terms relating to shapes generally, such as “circular” or “cylindrical” or “semi-circular” or “semi-cylindrical” or any related or similar terms, are not required to conform strictly to the mathematical definitions of circles or cylinders or other structures, but can encompass structures that are reasonably close approximations.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

The terms “approximately,” “about,” and “substantially” as used herein represent an amount close to the stated amount that still performs a desired function or achieves a desired result. For example, in some embodiments, as the context may dictate, the terms “approximately”, “about”, and “substantially” may refer to an amount that is within less than or equal to 10% of the stated amount. The term “generally” as used herein represents a value, amount, or characteristic that predominantly includes or tends toward a particular value, amount, or characteristic. As an example, in certain embodiments, as the context may dictate, the term “generally parallel” can refer to something that departs from exactly parallel by less than or equal to 20 degrees.

Unless otherwise explicitly stated, articles such as “a” or “an” should generally be interpreted to include one or more described items. Accordingly, phrases such as “a device configured to” are intended to include one or more recited devices. Such one or more recited devices can be collectively configured to carry out the stated recitations. For example, “a processor configured to carry out recitations A, B, and C” can include a first processor configured to carry out recitation A working in conjunction with a second processor configured to carry out recitations B and C.

The terms “comprising,” “including,” “having,” and the like are synonymous and are used inclusively, in an open-ended fashion, and do not exclude additional elements, features, acts, operations, and so forth. Likewise, the terms “some,” “certain,” and the like are synonymous and are used in an open-ended fashion. Also, the term “or” is used in its inclusive sense (and not in its exclusive sense) so that when used, for example, to connect a list of elements, the term “or” means one, some, or all of the elements in the list.

Overall, the language of the claims is to be interpreted broadly based on the language employed in the claims. The language of the claims is not to be limited to the non-exclusive embodiments and examples that are illustrated and described in this disclosure, or that are discussed during the prosecution of the application.

Although systems and methods for and of making liposomes, including control and targeted liposomes, have been disclosed in the context of certain embodiments and examples, this disclosure extends beyond the specifically disclosed embodiments to other alternative embodiments and/or uses of the embodiments and certain modifications and equivalents thereof. Various features and aspects of the disclosed embodiments can be combined with or substituted for one another in order to form varying modes of systems and methods for and of making liposomes, including control and targeted liposomes. The scope of this disclosure should not be limited by the particular disclosed embodiments described herein.

Certain features that are described in this disclosure in the context of separate implementations can be implemented in combination in a single implementation. Conversely, various features that are described in the context of a single implementation can be implemented in multiple implementations separately or in any suitable subcombination. Although features may be described herein as acting in certain combinations, one or more features from a claimed combination can, in some cases, be excised from the combination, and the combination may be claimed as any subcombination or variation of any subcombination.

While the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various embodiments described and the appended claims. Further, the disclosure herein of any particular feature, aspect, method, property, characteristic, quality, attribute, element, or the like in connection with an embodiment can be used in all other embodiments set forth herein. Any methods disclosed herein need not be performed in the order recited. Depending on the embodiment, one or more acts, events, or functions of any of the algorithms, methods, or processes described herein can be performed in a different sequence, can be added, merged, or left out altogether (e.g., not all described acts or events are necessary for the practice of the algorithm). In some embodiments, acts or events can be performed concurrently, e.g., through multi-threaded processing, interrupt processing, or multiple processors or processor cores or on other parallel architectures, rather than sequentially. Further, no element, feature, block, or step, or group of elements, features, blocks, or steps, are necessary or indispensable to each embodiment. Additionally, all possible combinations, subcombinations, and rearrangements of systems, methods, features, elements, modules, blocks, and so forth are within the scope of this disclosure. The use of sequential, or time-ordered language, such as “then,” “next,” “after,” “subsequently,” and the like, unless specifically stated otherwise, or otherwise understood within the context as used, is generally intended to facilitate the flow of the text and is not intended to limit the sequence of operations performed. Thus, some embodiments may be performed using the sequence of operations described herein, while other embodiments may be performed following a different sequence of operations.

Moreover, while operations may be depicted in the drawings or described in the specification in a particular order, such operations need not be performed in the particular order shown or in sequential order, and all operations need not be performed, to achieve the desirable results. Other operations that are not depicted or described can be incorporated in the example methods and processes. For example, one or more additional operations can be performed before, after, simultaneously, or between any of the described operations. Further, the operations may be rearranged or reordered in other implementations. Also, the separation of various system components in the implementations described herein should not be understood as requiring such separation in all implementations, and it should be understood that the described components and systems can generally be integrated together in a single product or packaged into multiple products. Additionally, other implementations are within the scope of this disclosure.

Some embodiments have been described in connection with the accompanying figures. Certain figures are drawn and/or shown to scale, but such scale should not be limiting, since dimensions and proportions other than what are shown are contemplated and are within the scope of the embodiments disclosed herein. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, any methods described herein may be practiced using any device suitable for performing the recited steps.

The methods disclosed herein may include certain actions taken by a practitioner; however, the methods can also include any third-party instruction of those actions, either expressly or by implication. For example, actions such as “positioning an electrode” include “instructing positioning of an electrode.”

In summary, various embodiments and examples of systems and methods for and of making liposomes, including control and targeted liposomes, have been disclosed. Although the systems and methods for and of making liposomes, including control and targeted liposomes, have been disclosed in the context of those embodiments and examples, this disclosure extends beyond the specifically disclosed embodiments to other alternative embodiments and/or other uses of the embodiments, as well as to certain modifications and equivalents thereof. This disclosure expressly contemplates that various features and aspects of the disclosed embodiments can be combined with, or substituted for, one another. Thus, the scope of this disclosure should not be limited by the particular disclosed embodiments described herein, but should be determined only by a fair reading of the claims that follow.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 1 V” includes “1 V.” Phrases preceded by a term such as “substantially” include the recited phrase and should be interpreted based on the circumstances (e.g., as much as reasonably possible under the circumstances). For example, “substantially perpendicular” includes “perpendicular.” Unless stated otherwise, all measurements are at standard conditions including temperature and pressure.

What is claimed is:

1. A method of treating breast cancer in a patient, the method comprising:
inserting into the body of the patient a first quantity of an actively targeted liposome, the actively targeted liposome comprising:
a lipid bilayer forming a spherical shell, wherein the spherical shell defines an interior liposomal cavity;
estrone linked to a surface of the actively targeted liposome using cyanuric chloride;
a chemotherapeutic drug, wherein the chemotherapeutic drug comprises at least one of a hydrophilic chemotherapeutic drug contained within the interior liposomal cavity and a hydrophobic chemotherapeutic drug contained within the lipid bilayer of the actively targeted liposome;
wherein the actively targeted liposomes comprise sonosensitive large unilamellar vesicles;
allowing the actively targeted liposomes to circulate throughout a circulatory system of the patient for a time, wherein the time is sufficiently long to allow aggregation of a second quantity of the actively targeted liposomes at a treatment area comprising the breast cancer, wherein the second quantity of the actively targeted liposomes is therapeutically significant;
applying ultrasound to the treatment area comprising the breast cancer and critically disrupting a third quantity of the actively targeted liposome so that the chemotherapeutic drug is released in the treatment area.

2. The method of treating breast cancer in a patient of claim 1, wherein the ultrasound applied to the treatment area comprises a low frequency ultrasound.

3. The method of treating breast cancer in a patient of claim 2, wherein the low frequency ultrasound comprises a 20 kHz ultrasound having at a power density of one of 6.08 W/cm², 6.97 W/cm², and 11.83 W/cm².

4. The method of treating breast cancer in a patient of claim 2, wherein the low frequency ultrasound applied to the treatment area is applied for less than about 10 minutes.

5. The method of treating breast cancer in a patient of claim 1, wherein the ultrasound applied to the treatment area comprises a high frequency ultrasound.

6. The method of treating breast cancer in a patient of claim 5, wherein the high frequency ultrasound comprises a 1.07 MHz ultrasound having at a power density of one of 10.5 W/cm², 50.2 W/cm², and 173 W/cm².

7. The method of treating breast cancer in a patient of claim 5, wherein the high frequency ultrasound comprises a 3.24 MHz ultrasound having at a power density of one of 10.5 W/cm², 50.2 W/cm², and 173 W/cm².

8. The method of treating breast cancer in a patient of claim 5, wherein the high frequency ultrasound applied to the treatment area is applied for less than about 10 minutes.

9. A method of treating a cancer in a patient, the method comprising:
inserting a quantity of an actively targeted nanoparticle in a body of the patient, the actively targeted nanoparticle comprising a lipid bilayer and a drug formulation within the actively targeted nanoparticle, wherein at least a portion of an external surface of the lipid bilayer is functionalized with a targeting moiety;
allowing at least a portion of the quantity of the actively targeted nanoparticle to aggregate at a treatment site in the patient, wherein the treatment site comprises the cancer;
after allowing at least a portion of the quantity of the actively targeted nanoparticle to aggregate at the treatment site in the patient, applying ultrasound to the treatment site in the patient.

10. The method of treating a cancer in a patient of claim 9, wherein the drug formulation comprises a hydrophobic drug contained within the lipid bilayer.

11. The method of treating a cancer in a patient of claim 9, wherein the drug formulation comprises a hydrophilic drug contained in a core of the actively targeted nanoparticle.

12. The method of treating a cancer in a patient of claim 9, wherein the targeting moiety comprises an estrogen hormone.

13. The method of treating a cancer in a patient of claim 12, wherein the estrogen hormone comprises estrone.

14. The method of treating a cancer in a patient of claim 9, wherein the targeting moiety comprises at least one of RGD, transferrin, and plasminogen.

15. The method of treating a cancer in a patient of claim 9, wherein the actively targeted nanoparticle is PEGylated.

16. The method of treating a cancer in a patient of claim 9, wherein applying ultrasound to the treatment site in the patient induces critical disruption of the lipid bilayer of a portion of the quantity of the actively targeted nanoparticle aggregated at the treatment site in the patient thereby releasing the drug formulation contained within the portion of the quantity of the actively targeted nanoparticle aggregated at the treatment site.

17. The method of treating a cancer in a patient of claim 9, wherein the ultrasound applied to the treatment site comprises a low frequency ultrasound applied for less than about 20 minutes.

18. The method of treating a breast cancer in a patient of claim 17, wherein the low frequency ultrasound comprises a 20 kHz ultrasound having at a power density of one of 6.08 W/cm², 6.97 W/cm², and 11.83 W/cm².

19. The method of treating a breast cancer in a patient of claim 9, wherein the ultrasound applied to the treatment site comprises a high frequency ultrasound applied for less than about 20 minutes.

20. The method of treating a breast cancer in a patient of claim 19, wherein the high frequency ultrasound comprises a 1.07 MHz ultrasound having at a power density of one of 10.5 W/cm², 50.2 W/cm², and 173 W/cm².

21. The method of treating a breast cancer in a patient of claim 19, wherein the high frequency ultrasound comprises a 3.24 MHz ultrasound having at a power density of one of 10.5 W/cm², 50.2 W/cm², and 173 W/cm².
